SEQUENCE LISTING (61 sequences)

SEQ ID NO: 1 - protein, 11 aa, synthetic construct
RASSSVSYIHW

SEQ ID NO: 2 - protein, 7 aa, synthetic construct
ATSNLAS

SEQ ID NO: 3 - protein, 9 aa, synthetic construct
QQWTSNPPT

SEQ ID NO: 4 - protein, 5 aa, synthetic construct
SYNMH

SEQ ID NO: 5 - protein, 17 aa, synthetic construct
AIYPGNGDTSYNQKFKG

SEQ ID NO: 6 - protein, 12 aa, synthetic construct
STYYGGDWYFNV

SEQ ID NO: 7 - protein, 9 aa, synthetic construct
RASSSVSYM

SEQ ID NO: 8 - protein, 7 aa, synthetic construct
APSNLAS

SEQ ID NO: 9 - protein, 9 aa, synthetic construct
QQWSFNPPT

SEQ ID NO: 10 - protein, 10 aa, synthetic construct
GYTFTSYNMH

SEQ ID NO: 11 - protein, 17 aa, synthetic construct
AIYPGNGDTSYNQKFKG

SEQ ID NO: 12 - protein, 13 aa, synthetic construct
VVYYSNSYWYFDV

SEQ ID NO: 13 - protein, 121 aa, synthetic construct [sequence omitted]

SEQ ID NO: 14 - protein, 106 aa, synthetic construct [sequence omitted]

SEQ ID NO: 15 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 16 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 17 - protein, 122 aa, synthetic construct [sequence omitted]

SEQ ID NO: 18 - protein, 106 aa, synthetic construct [sequence omitted]

SEQ ID NO: 19 - protein, 120 aa, synthetic construct
QLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 20 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 21 - protein, 118 aa, synthetic construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 22 - protein, 107 aa, synthetic construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 23 - protein, 118 aa, synthetic construct
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSS

SEQ ID NO: 24 - protein, 106 aa, synthetic construct
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIK

SEQ ID NO: 25 - protein, 121 aa, synthetic construct
QVQLQQSGAEVKKPGSSVKVSCKASGYTFTSYNMHWVKQAPGQGLEWIGAIYPGMGDTSYNQKFKGKATLTADESTNTAYMELSSLRSEDTAFYYCARSTYYGGDWYFDVWGQGTTVTVSS

SEQ ID NO: 26 - protein, 106 aa, synthetic construct
DIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIK

SEQ ID NO: 27 - protein, 122 aa, synthetic construct
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 28 - protein, 107 aa, synthetic construct; identical to SEQ ID NO: 20

SEQ ID NO: 29 - protein, 122 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 30 - protein, 107 aa, synthetic construct; identical to SEQ ID NO: 20

SEQ ID NO: 31 - protein, 125 aa, synthetic construct
EVQLVQSGGGLVHPGGSLRLSCTGSGFTFSYHAMHWVRQAPGKGLEWVSIIGTGGVTYYADSVKGRFTISRDNVKNSLYLQMNSLRAEDMAVYYCARDYYGAGSFYDGLYGMDVWGQGTTVTVSS

SEQ ID NO: 32 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSDWPLTFGGGTKVEIK

SEQ ID NO: 33 - protein, 123 aa, synthetic construct
AVQLVESGGGLVQPGRSLRLSCAASGFTFGDYTMHWVRQAPGKGLEWVSGISWNSGSIGYADSVKGRFTISRDNAKNSLYLQMNSLRAEDTALYYCTKDNQYGSGSTYGLGVWGQGTLVTVSS

SEQ ID NO: 34 - protein, 110 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKIHI

SEQ ID NO: 35 - protein, 6 aa, synthetic construct
QSVSSY

SEQ ID NO: 36 - skipped

SEQ ID NO: 37 - protein, 9 aa, synthetic construct
QQRSNWPIT

SEQ ID NO: 38 - protein, 8 aa, synthetic construct
GFTFHDYA

SEQ ID NO: 39 - protein, 8 aa, synthetic construct
ISWNSGTI

SEQ ID NO: 40 - protein, 15 aa, synthetic construct
AKDIQYGNYYYGMDV

SEQ ID NO: 41 - protein, 8 aa, synthetic construct
GFTFNDYA

SEQ ID NO: 42 - protein, 8 aa, synthetic construct
ISWNSGSI

SEQ ID NO: 43 - protein, 9 aa, synthetic construct
QQRSDWPLT

SEQ ID NO: 44 - protein, 8 aa, synthetic construct
GFTFSYHA

SEQ ID NO: 45 - protein, 7 aa, synthetic construct
IGTGGVT

SEQ ID NO: 46 - protein, 19 aa, synthetic construct
ARDYYGAGSFYDGLYGMDV

SEQ ID NO: 47 - protein, 9 aa, synthetic construct
QQRSNWPLT

SEQ ID NO: 48 - protein, 8 aa, synthetic construct
GFTFGDYT

SEQ ID NO: 49 - protein, 16 aa, synthetic construct
TKDNQYGSGSTYGLGV

SEQ ID NO: 50 - protein, 10 aa, synthetic construct
SASSSVSYMY

SEQ ID NO: 51 - protein, 7 aa, synthetic construct
RTSNLAS

SEQ ID NO: 52 - protein, 9 aa, synthetic construct
QQYHFFPPT

SEQ ID NO: 53 - protein, 7 aa, synthetic construct
GYTFINY

SEQ ID NO: 54 - protein, 6 aa, synthetic construct
LPGSHT

SEQ ID NO: 55 - protein, 12 aa, synthetic construct
GGTRASFYYFDY

SEQ ID NO: 56 - protein, 15 aa, synthetic construct
RASKSVSTSGYSYMH

SEQ ID NO: 57 - protein, 7 aa, synthetic construct
LASNLES

SEQ ID NO: 58 - protein, 9 aa, synthetic construct
QHSRELPYT

SEQ ID NO: 59 - protein, 7 aa, synthetic construct
GYTFTNY

SEQ ID NO: 60 - protein, 6 aa, synthetic construct
YPGDGD

SEQ ID NO: 61 - protein, 13 aa, synthetic construct
EPDDYDGYYYFDY

(19) United States
(12) Patent Application Publication
(10) Pub. No.: US 2026/0035476 A1
(43) Pub. Date: Feb. 5, 2026

(54) BIOMARKERS, DIAGNOSTIC METHODS, TREATMENTS, AND THERAPEUTICS FOR AUTOIMMUNE DISORDERS AND DISEASES

(71) Applicant: THE BOARD OF REGENTS OF THE UNIVERSITY OF OKLAHOMA, Norman, OK (US)

(72) Inventor: Carol WEBB, Oklahoma City, OK (US)

(73) Assignee: THE BOARD OF REGENTS OF THE UNIVERSITY OF OKLAHOMA, Norman, OK (US)

(21) Appl. No.: 18/997,568

(22) PCT Filed: Jul. 27, 2023
(86) PCT No.: PCT/US2023/071120

§ 371 Date: Jan. 22, 2025

Related U.S. Application Data
(60) Provisional application No. 63/393,065, filed on Jul. 28, 2022.
(60) Provisional application No. 63/433,913, filed on Dec. 20, 2022.

(51) Int. Cl. C07K 16/28; A61K 40/11; A61K 40/31; A61K 40/42; G01N 33/569

(52) U.S. Cl. CPC C07K 16/2896; A61K 40/11; A61K 40/31; A61K 40/4221; A61K 40/4224; C07K 16/2887; G01N 33/56972; A61K 2239/13; A61K 2239/29; C07K 2317/24; C07K 2317/31; C07K 2317/565; G01N 2333/70596

(57) ABSTRACT

Multispecific constructs, e.g., bispecific constructs, which comprise an anti-B cell binding arm that specifically binds to a B-cell-specific cell surface marker such as CD20, CD19, CD21, CD24, or CD38, and a second binding arm that specifically binds to CD68 protein, and optionally at least one additional binding arm, wherein the multispecific constructs bind specifically to naïve B cells which express transcription factor ARID3a. Methods of using the multispecific constructs to treat autoimmune disorders and/or diseases including but not limited to Systemic lupus erythematosus (SLE), and SLE flare-ups.

### CROSS REFERENCE TO RELATED APPLICATIONS

The present application is a U.S. National Phase application under 35 U.S.C § 371 of International Patent Application No. PCT/US2023/071120, filed on Jul. 27, 2023, and entitled “BIOMARKERS, DIAGNOSTIC METHODS, TREATMENTS, AND THERAPEUTICS FOR AUTOIMMUNE DISORDERS AND DISEASES,” which claims priority to U.S. Prov. Patent App. No. 63/393,065 filed on Jul. 28, 2022, and to U.S. Prov. Patent App. No. 63/433,913 filed on Dec. 20, 2022, all of which are expressly incorporated herein by reference in their entireties.

### STATEMENT REGARDING FEDERALLY SPONSORED RESEARCH AND DEVELOPMENT

This invention was made with government support under Contract Number AI118836 awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on Jan. 27, 2025, is named OKLAP0011US_ST26_1.xml and is 55,646 bytes in size.

### BACKGROUND

The transcription factor ARID3a is over-expressed in peripheral blood B cells, and the numbers of those ARID3a⁺ B cells are associated with increased disease (e.g., Systemic Lupus Erythematosus, a.k.a., SLE) activity. ARID3a expression occurs in only subsets of B cells within each differentiation stage.

Current therapies for autoimmune disorders such as SLE involve pan-B cell depletion and growth disruption and thus have broad undesirable side effects. Moreover, there has been no way to predict in advance which patients are about to undergo disease activity flare-ups. Improved treatments for SLE and other autoimmune disorders and diseases are urgently needed.

### BRIEF DESCRIPTION OF THE DRAWINGS

FIGS. 1A-1B shows that SLE naïve B cells co-express ARID3a and CD68. (FIG. 1A) Violin plots of scRNA-seq UMAP clusters of 10 SLE patient's sorted naïve B cells show ARID3a and CD68 transcripts in clusters 0.1 and 8. Dots are single cells. (FIG. 1B) Flow cytometry of SLE PBMCs gated on CD20⁺ B cells shows representative gating for CD27⁻IgD⁺ naïve B cells (SLE1) and analysis of CD68 and ARID3a expression (N=3). SLE2 (European American) and SLE1 (African A) show ARID3a and CD68 co-expression; SLE3 (EA) shows neither.

FIGS. 2A-2B shows results for magnetic bead-enriched total B cells that were stimulated with 1 μg/ml R848, 10 μg/ml F(ab′)2 anti-IgM, 10 ng/ml BAFF and IL21, 50 U/ml IL-2 and 20 ng/ml IFNγ for 3 days and analyzed by flow cytometry. (FIG. 2A) Resting and activated naïve B cells were gated (as per Jenks, S. A., et al., 2018. Distinct Effector B Cells Induced by Unregulated Toll-like Receptor 7 Contribute to Pathogenic Responses in Systemic Lupus Erythematosus. Immunity 49: 725-739 e726). (FIG. 2B) ARID3a cells are CD68-CD27⁺; ARID3a⁺CD68⁺ cells are CD27⁻IgD⁺ “naïve”. N=3

FIGS. 3A-3D shows results of a peripheral blood mononuclear cell (PBMC) sample obtained from a lupus patient that was stained by flow cytometry for total B cells (CD20⁺), T cells (CD3⁺), and naïve B cells (IgD⁺ CD27⁻). These subsets were analyzed for expression of CD68, and ARID3a, and for a novel anti-CD68/anti-CD20 bispecific construct of the present disclosure. T cells showed no expression of ARID3a or CD68 (FIG. 3A). B cells showed a sub-population of cells that co-stained with CD68 and ARID3a (29%, upper right-hand quadrant) (FIG. 3B). The bispecific construct did not stain T lymphocytes at all (FIG. 3C). The bispecific construct stains about 30% of the CD20⁺ naïve B cells, but does not react with all CD20⁺ B cells (FIG. 3D).

The following abbreviations may be used herein:
AAB: Autoantibody; AABs: Autoantibodies; ADC: antibody-drug conjugate; ANOVA: Analysis of Variance; ATCC: American Type Culture Collection; BSA: Bovine Serum Albumin; CD: cluster of differentiation; CDR: complementarity-determining region; CL or C_{L}: Constant light sequence (chain, domain); CH or C_{H}: Constant heavy sequence (chain, domain); CH1: Constant heavy chain 1; CH2: Constant heavy chain 2; CH3: Constant heavy chain 3; CHO: Chinese hamster ovary; dAb: domain antibody; DNA: deoxyribonucleic acid; EL: extracellular loop; ELISA: Direct Enzyme-Linked Immunosorbent Assay; Fab: antigen binding fragment; Fab′: antigen binding fragment prime; F(ab′): antigen binding prime fragment; F(ab′)₂: bivalent F(ab′) fragment; F(ab)₂: bivalent Fab fragment; Fe: constant fragment; FR: framework regions; Fv: Fragment variable region; Fc: Fragment crystallizable region; Fab: Fragment antigen binding region; Fd: Heavy chain of the Fab; HVR: hypervariable region; IgA: immunoglobulin alpha; IgD: immunoglobulin delta; IgE: immunoglobulin epsilon; IgG: immunoglobulin gamma; IgM: immunoglobulin mu; IMGT: International Immunogenetics Information System; Lc: Light chain of the Fab; mAb: monoclonal antibody; mRNA: messenger ribonucleic acid; NIBSC: National Institute for Biological Standards and Controls; NIMH: National Institutes of Mental Health; NT: N-Terminus; OUHSC: University of Oklahoma Health Sciences Center; PBMC: Peripheral blood mononuclear cells; PBS: Phosphate buffered saline; RT-qPCR: Real Time quantitative Polymerase Chain Reaction; scFv: single-chain variable fragment; sdFv: disulfide-linked variable fragments; SE: Standard error; S.E.M: Standard error of the mean; siRNA: short interfering ribonucleic acid; SLE: systemic lupus erythematosus; TM: transmembrane domain; tri-scFv: trivalent single-chain variable fragment; VL and V_{L}: Variable light sequence (chain, domain); VH and V_{H}: Variable heavy sequence (chain, domain).

### DETAILED DESCRIPTION

Enhanced disease activity in patients with systemic lupus erythematosus (SLE) appears to be associated with dramatic increase in numbers of B lymphocytes that express the transcription factor AT-rich-interactive domain 3a (ARID3a). New evidence presented herein demonstrates that production of pathogenic auto-antibodies (AABs) occurs in ARID3a⁺ B cells. It has been further discovered herein that protein CD68 is a surface marker that can be used in conjunction with other B cell markers (e.g., CD20) to recognize those AAB-producing B cells. Recognition of precursors to the AAB-producing cells has therapeutic use and can be used as a biomarker to predict oncoming disease activity. The marker disclosed herein, CD68, has heretofore been primarily known as a monocyte marker and has not previously been known to occur on this type of AAB-producing B cell.

Thus, the present disclosure is directed to therapeutic constructs that target ARID3a⁺ B cells which are enriched for AABs and display CD68 protein. Such therapeutics specifically targets both CD68 protein and B cells (e.g., which display CD20) can be used to attack and disable AAB-producing B cells, thereby reducing the numbers of such B cells and serving as a treatment for autoimmune disorders and diseases such as SLE and for complications thereof such as lupus nephritis, and for flares or flare-ups in subjects with SLE. The biomarkers can also be used in a diagnostic method for predicting disease flares, wherein when such a flare-up is predicted, the subject can be treated with the novel multispecific constructs of the present disclosure for preventing, mitigating, inhibiting, or ameliorating the effects of the flare-up.

In other embodiments, any of the therapeutic constructs described and/or claimed herein (e.g., bispecific constructs) can be used as treatments for autoimmune disorders and diseases, including but not limited to, acute or chronic Graft versus Host Disease (GvHD), Rheumatoid Arthritis (RA), Pemphigus Vulgaris, Chronic Lymphocytic Leukemia (CLL), Granulomatosis with Polyangiitis (GPA), Microscopic Polyangiitis (MPA), Multiple Sclerosis (MS), Primary Progressive Multiple Sclerosis (PPMS), Relapsing Multiple Sclerosis (RMS), Anti-neutrophil Cytoplasmic Antibody (ANCA) associated vasculitis, Non-Hodgkin's Lymphoma (NHL), Rassmussen's encephalitis, Immune Thrombocytopenic Purpura, Follicular Lymphoma Leukemia, and other immune conditions which can be treated by B cell depletion therapies (BCDT), Inflammatory Bowel Disease (IBD) such as Ulcerative colitis and Crohn's disease, Pediatric Acute-onset Neuropsychiatric Syndrome (PANS), and Pediatric Autoimmune Neuropsychiatric Disorder Associated with Streptoccocal Infections (PANDAS).

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning, and the embodiments and examples are meant to be exemplary, not exhaustive. Also, it is to be understood that the phraseology and terminology employed herein is for the purpose of description only and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. All of the compounds, compositions, and methods and application and uses thereof disclosed herein can be made and executed without undue experimentation in light of the present disclosure. Thus, while the compounds, compositions, and methods of the present disclosure have been described in terms of particular embodiments, it will be apparent to those of skill in the art that variations may be applied to the compounds, compositions, and methods and in the steps or in the sequence of steps of the methods described herein without departing from the concept, spirit, and scope of the inventive concepts.

All patents, applications, published patent applications, and non-patent publications including published articles mentioned in the specification or referenced in any portion of this application, including U.S. Prov. Patent App. No. 63/393,065 filed on Jul. 28, 2022, and U.S. Prov. Patent App. No. 63/433,913 filed on Dec. 20, 2022, are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. Where used herein, the specific term “single” is limited to only “one.”

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately,” where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. Further, all references to one or more embodiments or examples are to be construed as non-limiting to the claims.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds or conjugates of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

The term “active agent” as used herein is intended to refer to a substance which possesses a biological activity relevant to the present disclosure, and particularly refers to therapeutic and diagnostic substances which may be used in methods described in the present disclosure. By “biologically active” is meant the ability to modify the physiological system of a cell, tissue, or organism without reference to how the active agent has its physiological effects.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Non-limiting examples of animals or subjects within the scope and meaning of this term include dogs, cats, rats, mice, guinea pigs, chinchillas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable therapeutic or treatment effect in a subject without excessive adverse side effects (such as substantial toxicity, irritation, and allergic response) commensurate with a reasonable benefit/risk ratio when used in the manner of the present disclosure. The effective amount for a subject will depend upon the subject's type, size, and health, the nature and severity of the condition to be treated, the method of administration, the duration of treatment, the nature of concurrent therapy (if any), the specific formulations employed, and the like. The effective amount for a given situation can be determined by one of ordinary skill in the art using routine experimentation based on the information provided herein.

The terms “ameliorate” or “mitigate” mean that a detectable or measurable improvement occurs in a subject's condition, disease, or symptom thereof. A detectable or measurable improvement includes a subjective or objective decrease, reduction, inhibition, suppression, limit, or control in the occurrence, frequency, severity, progression, or duration of the condition or disease, or an improvement in a symptom or an underlying cause or a consequence of the disease, or a reversal of the disease. A successful treatment outcome can lead to a “therapeutic effect” or “benefit” of ameliorating, decreasing, reducing, inhibiting, suppressing, limiting, controlling, or preventing the occurrence, frequency, severity, progression, or duration of a disease or condition, or consequences of the disease or condition in a subject.

A decrease or reduction in worsening, such as stabilizing the condition or disease, is also a successful treatment outcome. A therapeutic benefit therefore need not be complete ablation or reversal of the disease or condition, or any one, most, or all adverse symptoms, complications, consequences, or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

As used herein the term “Systemic lupus erythematosus” (“SLE”) refers to a chronic autoimmune disorder in which the immune system attacks its own tissues, causing widespread inflammation and tissue damage in the affected organs, which may include the joints, skin, brain, lungs, kidneys, heart, blood vessels, and gastrointestinal system. SLE is characterized by disease flares, when the disease symptoms increase, and periods of remission, when symptoms improve and the subject feels better. Prior to the diagnostic methods of the present disclosure, disease flares have been unpredictable.

The terms “flare” or “flare-up” as used herein in reference to SLE refer to a measureable increase in disease activity in one or more organ systems involving new or worse clinical signs and symptoms and/or lab measurements. The increase must be considered clinically significant by an assessor, such as a physician or clinical researcher, and in most cases should prompt the consideration of a change or increase in treatment.

The term “naïve B cell” as used herein refers to a B cell lymphocyte that has matured and entered the bloodstream but has not yet been exposed to the antigen that is specific to its B cell receptor (BCR) and thus does not secrete antibodies.

The term “cluster of differentiation” or “CD” as used herein refers to any native CD protein from any vertebrate source, including mammals such as primates (e.g., humans) and rodents (e.g., mice and rats), unless otherwise indicated. The terms “anti-CD68 antibody” and “an antibody that binds to CD68” refer to an antibody or fragment thereof that is capable of binding to CD68 with sufficient affinity such that the antibody is useful as a diagnostic and/or therapeutic agent in targeting CD68. In one embodiment, the extent of binding of an anti-CD68 antibody to an unrelated, non-CD68 protein is less than about 10% of the binding of the antibody to CD68 as measured, e.g., by a radioimmunoassay (RIA).

As used herein, “CD68” (also known as GP110; SCARD1; and DKFZp686M 18236) means the human protein encoded by the gene identified by Entrez GeneID No. 968 and allelic variants thereof. As used herein, a “CD68-positive” cell is a cell wherein the presence of CD68 is detected by any appropriate analytical technique, such as immunohistochemistry.

CD20 is an activated-glycosylated phosphoprotein expressed on the surface of B-lymphocytes. CD20 includes, for example, human CD20 protein (see, e.g., NCBI RefSeq Nos. NP_068769.2 and NP_690605.1), which is 297 amino acids in length and may be generated, for example, from variant mRNA transcripts that lack a portion of the 5′ UTR (see, e.g., NCBI RefSeq No. NM_021950.3) or longer variant mRNA transcripts (see, e.g., NCBI RefSeq No. NM_152866.2). Antibody therapy with the antibody Rituximab, a chimeric anti-CD20 mAb was approved by FDA in 1997. Rituximab significantly improved all aspects of the survival statistics of B cell lymphoma and chronic lymphoid lymphoma (CLL) patients (Chu T W, Zhang R, Yang J, et al. A Two-Step Pretargeted Nanotherapy for CD20 Crosslinking May Achieve Superior Anti-Lymphoma Efficacy to Rituximab. Theranostics. 2015 Apr. 26; 5(8): 834-46).

The term “anti-CD20/anti-CD68 bispecific construct,” refers to a multispecific construct (e.g., a bivalent antibody) that is capable of binding to both CD20 and CD68 with sufficient affinity such that the construct is useful as a diagnostic and/or therapeutic agent in targeting CD20 and CD68 on AAB-producing B cells.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the term “specific binding” refers to binding where a molecule binds to a particular polypeptide or epitope on a particular polypeptide without substantially binding to any other polypeptide or polypeptide epitope.

The anti-CD68 multispecific constructs of the present disclosure, such as the anti-CD20/anti-CD68 bispecific construct for example, may be produced using recombinant methods and compositions, for example, as described in U.S. Pat. No. 4,816,567.

The term “antibody” as used herein can refer to both intact “full length” antibodies as well as to antigen-binding fragments thereof (unless otherwise explicitly noted). The aforementioned antigen-binding fragments may also be referred to herein as antigen binding fragments, antigen binding compounds, antigen binding portions, binding fragments, binding portions, or antibody fragments.

The terms “full-length antibody,” “intact antibody,” and “whole antibody” are used herein interchangeably to refer to an antibody having a structure substantially similar to a native antibody structure or having heavy chains that contain an Fc region as defined herein.

As used herein, the term “antibody” includes, but is not limited to, synthetic antibodies, monoclonal antibodies, recombinantly produced antibodies, intrabodies, multispecific antibodies (including bi-specific antibodies), human antibodies, humanized antibodies, chimeric antibodies, recombinant single chain polypeptide molecules in which light and heavy chain variable regions are connected by a peptide linker, i.e., single-chain Fv (scFv) fragments, bivalent scFv (bi-scFv), trivalent scFv (tri-scFv), Fab fragments, Fab′ fragments, F(ab′) fragments, F(ab′)₂ fragments, F(ab)₂ fragments, disulfide-linked Fvs (sdFv) (including bi-specific sdFvs), and anti-idiotypic (anti-Id) antibodies, dAb fragments, nanobodies, diabodies, triabodies, tetrabodies, linear antibodies, isolated CDRs, and epitope-binding fragments of any of the above. Regardless of structure, an antibody fragment refers to an isolated portion of the antibody that binds to the same antigen that is recognized by the intact antibody. For example, an anti-CD68 antibody fragment binds with an epitope of CD68 and an anti-CD20 antibody fragment binds with an epitope of CD20. Antibody fragments can be produced by recombinant DNA techniques or by enzymatic or chemical separation of intact immunoglobulins. Examples of antibody fragments include but are not limited to Fv, Fab, Fab′, Fab′-SH, F(ab′)2; diabodies; linear antibodies; single-chain antibody molecules (e.g., scFv); and multispecific antibodies formed from antibody fragments. The term “Fc region” herein is used to define a C-terminal region of an immunoglobulin heavy chain that contains at least a portion of the constant region. The term includes native sequence Fc regions and variant Fc regions.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003). Antibody fragments can be made by various techniques, including but not limited to proteolytic digestion of an intact antibody as well as production by recombinant host cells (e.g., E. coli or phage), as described herein.

As noted, the antibodies of several embodiments provided herein may be monospecific, bispecific, trispecific, or of greater multispecificity, such as multispecific antibodies formed from antibody fragments. As noted, the term “antibody” also includes a diabody (homodimeric Fv fragment) or a minibody (V_{L}—V_{H}—C_{H3}), a bispecific antibody, or the like. A bispecific or bifunctional antibody is an artificial hybrid antibody having two different heavy/light chain pairs and two different binding sites. Multispecific antibodies may be specific for different epitopes of a polypeptide or may be specific for both a polypeptide as well as for a heterologous epitope, such as a heterologous polypeptide or solid support material. Single chain antibodies produced by joining antibody fragments using recombinant methods, or a synthetic linker, are also encompassed by the present disclosure (e.g., see, for example, International Patent Application Publication Nos. WO 93/17715; WO 92/08802; WO 91/00360; and WO 92/05793; and U.S. Pat. Nos. 4,474,893; 4,714,681; 4,925,648; 5,573,920; and 5,601,819).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally-occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

In a non-limiting embodiment for example, monoclonal antibodies can be made by the hybridoma method first described by Kohler et al. (Nature, 256:495 (1975)), or may be made by recombinant DNA methods (see, for example, U.S. Pat. No. 4,816,567).

An “isolated” antibody refers to an antibody that has been identified and separated and/or recovered from components of its natural environment and/or an antibody that is recombinantly produced. A “purified antibody” is an antibody that is typically at least 50% w/w pure of interfering proteins and other contaminants arising from its production or purification but does not exclude the possibility that the monoclonal antibody is combined with an excess of pharmaceutical acceptable carrier(s) or other vehicle(s) intended to facilitate its use. Interfering proteins and other contaminants can include, for example, cellular components of the cells from which an antibody is isolated or recombinantly produced. Sometimes monoclonal antibodies are at least 60%, at least 70%, at least 80%, at least 90%, at least 95%, or at least 99% w/w pure of interfering proteins and contaminants from production or purification. The antibodies and antigen binding compounds described herein can be provided in isolated and/or purified form.

In at least certain embodiments of the present disclosure, the term “therapeutic agent” refers to an active agent comprising an antibody and/or antibody-derived compound or other compound as described herein.

A “diagnostic agent” is a substance that is useful in diagnosing a disease. Useful diagnostic agents of the present disclosure may include antibodies and antibody-derived compounds described herein, and may further comprise by linkage or other association radioisotopes, dyes, contrast agents, fluorescent compounds or molecules, and enhancing agents (e.g., paramagnetic ions).

An “immunoconjugate” or “antibody-drug conjugate” (ADC) is a conjugate of an antibody or antibody-derived compound with an atom, molecule, or a higher-ordered structure (e.g., with a liposome), a therapeutic agent, or a diagnostic agent.

As used herein, the term “antibody fusion protein” is a recombinantly produced antigen-binding molecule in which an antibody or antibody fragment is linked to another protein or peptide, such as the same or different antibody or antibody fragment. The fusion protein may comprise a single antibody component, a multivalent or multispecific combination of different antibody components, or multiple copies of the same antibody component, or other component described elsewhere herein. The fusion protein may additionally comprise an antibody or an antibody fragment and a therapeutic agent.

By “binding domain” is meant a part of a compound or a molecule that specifically binds to a target epitope, antigen, ligand, or receptor. Binding domains include but are not limited to antibodies (e.g., monoclonal, polyclonal, recombinant, humanized, and chimeric antibodies), antibody fragments or portions thereof (e.g., Fab fragments, Fab′2, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner.

The basic structural unit of a whole natural antibody is a tetramer of subunits. Each tetramer includes two identical pairs of polypeptide chains, each pair having one “light” chain (about 25 kDa) and one “heavy” chain (about 50-70 kDa). The amino-terminal portion of each chain includes a variable region (variable heavy chain and variable light chain) of about 100 to 120 or more amino acids, which include portions called complementarity determining regions (CDRs) as described below, which are primarily responsible for antigen recognition. A CDR is a segment of the variable region of an antibody that is complementary in structure to the epitope to which the antibody binds and is more variable than the rest of the variable region. Accordingly, a CDR is sometimes referred to as a hypervariable region (HVR). The three CDRs of the variable heavy chain may be referred to herein as CDRH1, CDRH2, and CDRH3. The three CDRs of the variable light chain may be referred to herein as CDRL1, CDRL2, and CDRL3. CDRs are also known as hypervariable regions (HVRs). This variable region is initially expressed linked to a cleavable signal peptide. The variable region without the signal peptide is sometimes referred to as a mature variable region. Thus, for example, a “light chain mature variable region” means a light chain variable region without the light chain signal peptide. The carboxy-terminal portion of each chain defines a constant region primarily responsible for effector function. CDR peptides can be obtained by constructing genes encoding the CDR of an antibody of interest.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three HVRs (i.e, CDRs). (See, e.g., Kindt et al., Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The CDRs (i.e., HVRs) form structurally defined loops (“hypervariable loops”) and/or contain the antigen-contacting residues (“antigen contacts”).

Unless otherwise indicated, CDRs and other residues in the variable domain (e.g., FR residues) are numbered herein according to the method of Kabat et al., (see below).

Light chains are classified as either kappa or lambda. Heavy chains are classified as gamma, mu, alpha, delta, or epsilon, and define the antibody's isotype as IgG, IgM, IgA, IgD, and IgE, respectively. Several of these may be further divided into subclasses (isotypes), e.g., IgG₁, IgG₂, IgG₃, IgG₄, IgA₁, and IgA₂. The term IgG “isotype” or “subclass” as used herein is meant any of the subclasses of immunoglobulins defined by the chemical and antigenic characteristics of their constant regions. Within light and heavy chains, the variable and constant regions are joined by a “J” region of about 12 or more amino acids, with the heavy chain also including a “D” region of about 10 or more amino acids.

The mature variable regions of each light/heavy chain pair form the antibody binding site. Thus, an intact antibody has two binding arms. Except in bifunctional or bispecific antibodies, the two binding arms are the same. The chains all exhibit the same general structure of relatively conserved framework regions (FR) joined by three hypervariable regions, which as noted above are known as CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3, and FR4.

Where used herein, the term “multispecific construct” refers to an antibody, or a portion or fragment of an antibody, which is able to bind to two or more (e.g., three, four, or five) different antigens with high specificity. Where used herein, the term “bispecific construct” refers to an antibody, or a portion or fragment of an antibody, which is able to bind to exactly two different antigens (e.g., CD20 and CD68) with high specificity. Where used herein, the term “trispecific construct” refers to an antibody, or a portion or fragment of an antibody, which is able to bind to exactly three different antigens (e.g., any combination of CD19, CD20, CD21, CD24, and CD38, plus CD68) with high specificity. The term “multispecific construct” as used herein may also refer to a CAR T-cell which is able to bind (via portions or fragments of antibodies) to two or more (e.g., three, four, or five) different antigens with high specificity. The multispecific construct may comprise a chimeric antigen receptor (CAR) T-cell. The CAR T-cell may further comprise a transmembrane domain and an intracellular signaling domain. The intracellular signaling domain may comprise a primary immune cell signaling sequence derived from the group CD3_{zeta}, CD3_{gamma}, CD3_{delta}, and CD3ₑₚₛᵢₗₒₙ. The intracellular signaling domain may further comprise a costimulatory signaling sequence derived from the group consisting of CD28, 4-1BB, ICOS, or OX40. In one embodiment, the intracellular signaling domain comprises a primary immune cell signaling sequence derived from CD3_{zeta} and a costimulatory signaling sequence derived from CD28.

The assignment of amino acids to each domain (FR1, CDR1, FR2, CDR2, FR3, CDR3, and FR4) is done in accordance with the protocols of the IMGT, e.g., see Ehrenmann, F., Kaas, Q. and Lefranc, M.-P., “IMGT/3Dstructure-DB and IMGT/DomainGapAlign: a database and tool for immunoglobulins or antibodies, T cell receptors, MHC, IgSF and MhcSF,” Nucl. Acids Res., 38(S1):D301-D307 (2010). DOI:10.1093/nar/kgp946. PMID:19900967, and Ehrenmann, F. and Lefranc, M.-P. Cold Spring Harb. Protocols, 2011(6):737-749. DOI:10.1101/pdb.prot5636. PMID:21632775. Using these IMGT protocols, CDR sequences are determined from the amino acid sequences of each of the mAbs B4C, 42.4.1, and 3C3.1, described elsewhere in the present disclosure, and are hereby incorporated by reference herein in their entireties.

In other embodiments, the assignment of amino acids to each domain may be done in accordance with the protocols of Kabat (Sequences of Proteins of Immunological Interest, National Institutes of Health, Bethesda, Md., 1987 and 1991), or Chothia & Lesk (J. Mol. Biol. 196:901-917 (1987); Chothia et al., Nature, 342:878-883 (1989)). Kabat also provides a widely used numbering convention (Kabat numbering) in which corresponding residues between different heavy chains or between different light chains are assigned the same number. Using these protocols, CDR sequences can be determined from the amino acid sequences of a VL or VH region of an antibody.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., “Epitope Mapping Protocols,” in Methods in Molecular Biology, Vol. 66, Glenn E. Morris, Ed. (1996).

Also within the scope of the present disclosure are antibodies or antibody-derived compounds thereof in which specific amino acids have been substituted, deleted, or added. These alternations do not have a substantial effect on the peptide's biological properties, such as (but not limited to) binding activity. For example, antibodies may have amino acid substitutions in the framework region, such as to improve binding to the antigen. In another example, a selected, small number of acceptor framework residues can be replaced by the corresponding donor amino acids. The donor framework can be a mature or germline human antibody framework sequence or a consensus sequence. Guidance concerning how to make phenotypically silent amino acid substitutions is provided in the following: Bowie et al. (Science, 247: 1306-1310 (1990)); Cunningham et al. (Science, 244: 1081-1085 (1989)); Ausubel (ed.) (Current Protocols in Molecular Biology, John Wiley and Sons, Inc. (1994)); Maniatis et al. (Molecular Cloning: A Laboratory Manual, Cold Spring Harbor laboratory, Cold Spring Harbor, N.Y. (1989)); Pearson (Methods Mol. Biol. 243:307-31 (1994)); and Gonnet et al. (Science, 256:1443-45 (1992)).

For purposes of classifying amino acids substitutions as conservative or nonconservative, amino acids are grouped in one non-limiting embodiment as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same group. Non-conservative substitutions constitute exchanging a member of one of these groups for a member of another.

Tables of conservative amino acid substitutions have been constructed and are known in the art. In other embodiments, examples of interchangeable amino acids include, but are not limited to, the following: arginine and lysine; glutamate and aspartate; serine and threonine; glutamine and asparagine; and valine, leucine, and isoleucine. In other non-limiting embodiments, the following substitutions can be made: Ala (A) by leu, ile, or val; Arg (R) by gln, asn, or lys; Asn (N) by his, asp, lys, arg, or gln; Asp (D) by asn or glu; Cys (C) by ala or ser; Gln (Q) by glu or asn; Glu (E) by gln or asp; Gly (G) by ala; His (H) by asn, gln, lys, or arg; Ile (I) by val, met, ala, phe, or leu; Leu (L) by val, met, ala, phe, or ile; Lys (K) by gln, asn, or arg; Met (M) by phe, ile, or leu; Phe (F) by leu, val, ile, ala, or tyr; Pro (P) by ala; Ser (S) by thr; Thr (T) by ser; Trp (W) by phe or tyr; Tyr (Y) by trp, phe, thr, or ser; and Val (V) by ile, leu, met, phe, or ala.

Other considerations for amino acid substitutions include whether or not the residue is located in the interior of a protein or is solvent- (i.e., externally) exposed. For interior residues, conservative substitutions include for example: Asp and Asn; Ser and Thr; Ser and Ala; Thr and Ala; Ala and Gly; Ile and Val; Val and Leu; Leu and Ile; Leu and Met; Phe and Tyr; and Tyr and Trp. For solvent-exposed residues, conservative substitutions include for example: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; and Phe and Tyr.

Percentage sequence identities can be determined with antibody sequences maximally aligned by the Kabat numbering convention described above. After alignment, if a particular antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises an antibody may contain the antibody alone or in combination with other ingredients. The phrase “pharmaceutically acceptable salt” refers to pharmaceutically acceptable organic or inorganic salts of a presently-disclosed antibody, or binding fragment, or conjugate thereof, or agent administered with presently-disclosed antibody or fragment or conjugaste thereof. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as (but not limited to) an acetate ion, a succinate ion, or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterions.

A “human antibody” is one which possesses an amino acid sequence which corresponds to that of an antibody produced by a human or a human cell or derived from a non-human source that utilizes human antibody repertoires or other human antibody-encoding sequences. Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A chimeric antibody is a molecule in which different portions are derived from different animal species. For example, an antibody may contain a variable region or just the CDRs derived from a murine mAb and a human immunoglobulin constant region. Chimeric antibodies can be produced by recombinant DNA techniques, e.g., see Morrison et al. (Proc Natl Acad Sci, 81:6851-6855 (1984)). For example, a gene encoding a murine (or other species) monoclonal antibody molecule is digested with restriction enzymes to remove the region encoding the murine Fc, and the equivalent portion of a gene encoding a human Fc constant region is substituted. Chimeric antibodies can also be created by recombinant DNA techniques where DNA encoding murine variable regions can be ligated to DNA encoding the human constant regions, e.g., see International Patent Publication Nos. WO 87/002671 and WO 86/01533, and U.S. Pat. No. 4,816,567.

A chimeric antibody is a recombinant protein that contains the variable domains including the CDRs of an antibody derived from one species, for example a rodent or rabbit antibody, while the constant domains of the antibody molecule are generally derived from those of a human antibody. For veterinary applications, the constant domains of the chimeric antibody may be derived from that of other species, such as but not limited to, a cat, dog, or horse.

The term “chimeric” when used herein in reference to a multispecific construct refers to a construct which is constructed amino to comprise antibody components from two or more species. In some embodiments, the multispecific construct is humanized. In some embodiments, the multispecific construct is chimeric. A humanized antibody is by definition chimeric, since it comprises portions of a human antibody and portions of a non-human antibody (e.g., mouse, rat rabbit, etc.) In some embodiments, the multispecific construct comprises an antibody fragment. In some embodiments, the antibody fragment is selected from the group consisting of Fab, Fab′-SH, Fv, scFv, and (Fab′)₂ fragments. In some embodiments, the multispecific construct comprises a full-length antibody. In some embodiments, the multispecific construct comprises at least a portion of an IgG antibody.

A chimeric antibody can be humanized by replacing the sequences of, for example, a murine FR in the variable domains of the chimeric antibody with one or more different human FR sequences. Specifically, mouse CDRs are transferred from heavy and light variable chains of the mouse immunoglobulin into the corresponding variable domains of a human antibody. As simply transferring mouse CDRs into human FRs may result in a reduction of antibody affinity, additional modifications might be required in order to restore the original affinity of the murine antibody. This can be accomplished by the replacement of one or more human residues in the FR regions with their murine counterparts to obtain an antibody with enhanced binding affinity to the desired epitopes (e.g., see Tempest et al. (Biotechnology, 9:266 (1991)) and Verhoeyen et al. (Science, 239: 1534 (1988))). Techniques for producing humanized antibodies are known to persons having ordinary skill in the art and are disclosed, for example, by Jones et al. (Nature, 321: 522 (1986)), Riechmann et al. (Nature, 332: 323 (1988)), Verhoeyen et al. (Science, 239: 1534 (1988)), Carter et al. (Proc. Nat'l Acad. Sci. USA, 89: 4285 (1992)), Sandhu (Crit. Rev. Biotech. 12:437 (1992)), and Singer et al. (J. Immun. 150: 2844 (1993)), Almagro and Fransson, Front. Biosci. 13:1619-1633 (2008), and are further described, e.g., in Queen et al., Proc. Natl Acad. Sci. USA 86:10029-10033 (1989); Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

As noted elsewhere, in some embodiments, the multispecific construct is humanized. A “humanized” antibody refers to a chimeric antibody comprising amino acid residues from non-human CDRs and amino acid residues from human framework regions (FRs). In certain embodiments, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

As previously noted, an antibody's light chain variable region contains an FR interrupted by three different CDRs (CDRL1, CDRL2, and CDRL3), and a heavy chain variable region contains an FR interrupted by three different CDRs (CDRH1, CDRH2, and CDRH3). In one non-limiting embodiment, humanized antibodies are antibody molecules from non-human species having one, two, three, four, five or all six CDRs from the non-human species and a framework region from a human immunoglobulin molecule.

A humanized antibody is a genetically engineered antibody in which the variable heavy and variable light CDRs from a non-human “donor” antibody are grafted into human “acceptor” antibody sequences (see for example, U.S. Pat. Nos. 5,530,101; 5,585,089; 5,225,539; 6,407,213; 5,859,205; and 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a non-human donor antibody and variable region framework sequences and constant regions, if present, entirely or substantially from human antibody sequences. Similarly, a humanized heavy chain has at least one, two, and usually all three CDRs entirely or substantially from a donor antibody heavy chain, and a heavy chain variable region framework sequence, and heavy chain constant region, if present, substantially from human heavy chain variable region framework and constant region sequences. Similarly, a humanized light chain has at least one, two, and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain.

As noted, humanized antibodies can be generated by replacing framework sequences of the variable region that are not directly involved in antigen binding with equivalent sequences from human variable regions. Those methods include isolating, manipulating, and expressing the nucleic acid sequences that encode all or part of variable regions from at least one of a heavy or light chain. The recombinant DNA encoding the humanized antibody, or fragment thereof, can then be cloned into an appropriate expression vector. Each antibody light and heavy chain variable region consists of a framework region interrupted by the three corresponding CDRs. In one non-limiting embodiment, humanized antibodies are antibody molecules from non-human species having one, two, or all CDRs from the non-human species and a framework region from a human immunoglobulin molecule.

The humanized antibodies of the present disclosure can be produced by methods well known in the art. For example, once an antibody is obtained, the variable regions can be sequenced, and the location of the CDRs and framework residues determined using the methods of, for example, Kabat et al. (Sequences of Proteins of Immunological Interest, Fifth Edition, U.S. Department of Health and Human Services, NIH Publication No. 91-3242 (1991)); and Chothia et al. (J. Mol. Biol., 196:901-917 (1987)). The light and heavy chain variable regions can optionally be ligated to corresponding constant regions. CDR-grafted antibody molecules can be produced by CDR-grafting or CDR substitution. One, two, or all CDRs of an immunoglobulin chain can be replaced. For example, all of the CDRs of a particular antibody may be from at least a portion of a non-human animal (e.g., mouse, such as (but not limited to) CDRs shown herein), or only some of the CDRs may be replaced. It is only necessary to keep the CDRs which are required for specific and high binding affinity of the antibody to the target antigen. Once expressed, antibodies can be purified according to standard procedures of the art, including but not limited to HPLC purification, column chromatography, and gel electrophoresis. Methods for producing human antibodies include, but are not limited to, those shown in U.S. Pat. Nos. 4,634,664; 4,634,666; 5,877,397; 5,874,299; 5,814,318; 5,789,650; 5,770,429; 5,661,016; 5,633,425; 5,625,126; 5,569,825; 5,545,806; 5,877,218; 5,871,907; 5,858,657; 5,837,242; 5,733,743; 5,565,332; 5,821,337, 7,527,791, 6,982,321, and 7,087,409; and International Patent Application Publication Nos. WO 91/17271; WO 92/01047; and WO93/12227.

Human framework regions that may be used for humanization include but are not limited to: framework regions selected using the “best-fit” method (see, e.g., Sims et al., J. Immunol. 151:2296 (1993)); framework regions derived from the consensus sequence of human antibodies of a particular subgroup of light or heavy chain variable regions (see, e.g., Carter et al., Proc. Nat. Acad. Sci. USA, 89:4285 (1992); and Presta et al., J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In certain instances, a multispecific construct of the present disclosure may be dertived from portions of a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008). Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extra-chromosomally or integrated randomly into the animal's chromosomes. In such transgenic mice, the endogenous immunoglobulin loci have generally been inactivated. Methods for obtaining human antibodies from transgenic animals, are shown for example, in Lonberg, Nat. Biotech. 23:1117-1125 (2005), U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can be made by hybridoma-based methods. Human myeloma and mouse-human heteromyeloma cell lines for the production of human monoclonal antibodies have been described, (e.g., see Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain.

In some instances, the methods described herein include administering the multispecific construct in combination with an additional therapeutic agent (e.g., an antibody-drug conjugate (ADC), or an anti-inflammatory agent, e.g., prednisolone.

A fully human antibody can be obtained from a transgenic non-human animal (see, e.g., Mendez et al. (Nature Genetics, 15: 146-156, 1997); and U.S. Pat. No. 5,633,425). Methods for producing fully human antibodies using either combinatorial approaches or transgenic animals transformed with human immunoglobulin loci are known in the art (e.g., Mancini et al. (New Microbiol., 27:315-28 (2004)); Conrad and Scheller (Comb. Chem. High Throughput Screen. 8:117-26 (2005)); and Brekke and Loset (Curr. Opin. Pharmacol., 3:544-50 (2003)). Such fully human antibodies are expected to exhibit even fewer side effects than chimeric or humanized antibodies and to function in vivo as essentially endogenous human antibodies. In certain non-limiting embodiments, the claimed methods and procedures may utilize human antibodies produced by such techniques.

A CDR in a humanized or human antibody may be defined as “substantially derived from” or “substantially identical to” a corresponding CDR in a non-human antibody when at least 60%, or at least 65%, or at least 70%, or at least 75%, or at least 80%, or at least 85%, or at least 90%, or at least 95%, or 100% of corresponding residues (as defined by Kabat) are identical between the respective CDRs. In some non-limiting embodiments, a CDR in a humanized antibody or human antibody is substantially derived from or substantially identical to a corresponding CDR in a non-human antibody when there are no more than one, two, or three conservative amino acid substitutions in any given CDR. The variable region framework sequences of an antibody chain or the constant region of an antibody chain are “substantially from” a human variable region framework sequence or human constant region, respectively, when at least about 60%, at least about 65%, at least about 70%, at least about 75%, at least about 80%, at least about 85%, at least about 90%, at least about 95%, or about 100% of corresponding residues (as defined by Kabat numbering) are identical. As noted elsewhere herein, although humanized antibodies often incorporate all six CDRs (e.g., as defined elsewhere herein) from a non-human (e.g., mouse or rabbit) antibody, they can also be made with less than all of the non-human CDRs (e.g., at least 2, 3, 4, or 5).

The present disclosure describes multispecific constructs that contain at least a first antigen-binding domain and a second antigen-binding domain, methods of producing the multispecific constructs, methods of treating a disease or disorder using the multispecific constructs, polynucleotides encoding the multispecific constructs, vectors and host cells containing said polynucleotides, and compositions and pharmaceutical compositions comprising the multispecific constructs.

An example of a multispecific construct is a bispecific antibody construct, such as an IgG-like bispecific antibody having one arm which binds to a target A (e.g., CD20) and another arm which binds to a target B (e.g., CD68). Structurally it may be made from half of the anti-A antibody and half of the anti-B antibody, with the similar size and shape as a natural IgG. In order to facilitate downstream development, it is desired that such bispecific molecules can be easily produced like normal IgG from a single host cell with high expression level and correctly assembled form.

The present disclosure provides, in certain non-limiting embodiments, a multispecific construct comprising a binding arm which binds to a CD protein of the present disclosure (e.g., CD20, CD19, CD21, CD24, CD38, and CD68) in which the heavy chain variable region has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to a heavy chain variable sequence disclosed herein, and the light chain variable region has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to a light chain variable sequence disclosed herein. In some aspects, the antibody is a humanized antibody, and there is at least one murine backmutation in the heavy chain variable framework region. In other aspects, the antibody is a humanized antibody, and there is at least one murine backmutation in the light chain variable framework region.

Additionally, the disclosure provides, in certain non-limiting embodiments, a multispecific construct comprising a binding arm which binds to a CD protein of the present disclosure (e.g., CD19, CD20, CD21, CD24, CD38, and CD68) in which the humanized heavy chain variable region comprises CDRs having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to the CDRs of a heavy chain variable sequence disclosed herein, and the humanized light chain variable region comprises CDRs having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to the CDRs of a light chain variable sequence disclosed herein.

Heavy and light chain variable regions of humanized antibodies can be linked to at least a portion of a human constant region, for example, for human antibody isotypes IgG1, IgG2, IgG3, or IgG4. Light chain constant regions can be lambda or kappa. Antibodies can be expressed as, for example (but not by way of limitation): tetramers containing two light and two heavy chains, as separate heavy chains, light chains, as Fab, Fab′, F(ab′)₂, and Fv, or as single chain antibodies in which heavy and light chain variable domains are linked through a spacer. All antibody isotypes are encompassed by the present disclosure, including (but not limited to) IgG (e.g., IgG1, IgG2, IgG3, IgG4), IgM, IgA (IgA1, IgA2), IgD, or IgE. The antibodies or antigen-binding portions thereof may be mammalian (e.g., mouse, rabbit, human) antibodies or antigen-binding portions thereof.

Humanized or chimeric antibodies are typically produced by recombinant expression. Recombinant polynucleotide constructs typically include an expression control sequence operably linked to the coding sequences of antibody chains, including naturally-associated or heterologous promoter regions. The expression control sequences may be eukaryotic promoter systems in vectors capable of transforming or transfecting eukaryotic host cells. Once the vector has been incorporated into the appropriate host, the host is maintained under conditions suitable for high level expression of the nucleotide sequences, and for the collection and purification of the crossreacting antibodies.

Mammalian cells may be used as hosts for expressing nucleotide segments encoding immunoglobulins or fragments thereof. A number of suitable host cell lines capable of secreting intact heterologous proteins have been developed in the art, and include (but are not limited to) CHO cell lines (e.g., DG44), various COS cell lines, HeLa cells, HEK293 cells, L cells, and non-antibody-producing myelomas including Sp2/0 and NS0. In certain particular (but non-limiting) embodiments, the cells are non-human. Expression vectors for these cells can include expression control sequences, such as (but not limited to) an origin of replication, a promoter, an enhancer, and necessary processing information sites, such as (but not limited to) ribosome binding sites, RNA splice sites, polyadenylation sites, and transcriptional terminator sequences. Examples of expression control sequences include, but are not limited to, promoters derived from endogenous genes, cytomegalovirus, SV40, adenovirus, or bovine papillomavirus.

The present disclosure also encompasses nucleic acids encoding the multispecific constructs or antigen-binding portions thereof. The nucleic acid may be expressed in a cell to produce the presently disclosed construct or antigen-binding portion thereof. The isolated nucleic acids of the present disclosure comprises, for example (but not by way of limitation), sequences encoding peptides that are at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% homologous to the construct sequences described elsewhere herein.

A nucleic acid encoding the present constructs or antigen-binding portions thereof may be introduced into an expression vector that can be expressed in a suitable expression system, followed by isolation or purification of the expressed antibody or antigen-binding portion thereof. Optionally, a nucleic acid encoding the present construct or antigen-binding portion thereof can be translated in a cell-free translation system, e.g., see U.S. Pat. No. 4,816,567.

The constructs or antigen binding fragments thereof can be produced by host cells transformed with DNA encoding light and heavy chains (or CDR portions thereof) of a desired antibody. The constructs can be isolated and purified from these culture supernatants and/or cells using standard techniques. For example, a host cell may be transformed with DNA encoding the light chain, the heavy chain, or both, of a construct. Recombinant DNA technology may also be used to remove some or all of the DNA encoding either or both of the light and heavy chains that is not necessary for binding, e.g., the constant region.

The present nucleic acids can be expressed in various suitable cells, including prokaryotic and eukaryotic cells, e.g., bacterial cells, (e.g., E. coli), yeast cells, plant cells, insect cells, and mammalian cells. A number of mammalian cell lines are known in the art and include (but are not limited to) immortalized cell lines available from the ATCC. Non-limiting examples of the cells include all cell lines of mammalian origin or mammalian-like characteristics, including but not limited to, parental cells, derivatives and/or engineered variants of monkey kidney cells (COS, e.g., COS-1, COS-7), HEK293, baby hamster kidney (BHK, e.g., BHK21), CHO, NS0, PerC6, BSC-1, human hepatocellular carcinoma cells (e.g., Hep G2), SP2/0, HeLa, Madin-Darby bovine kidney (MDBK) cells, myeloma cells, and lymphoma cells. The engineered variants include (for example but not by way of limitation) glycan profile modified and/or site-specific integration site derivatives.

The present disclosure also provides for cells comprising the nucleic acids described herein. The cells may be a hybridoma or transfectant. Examples of the cell types are discussed elsewhere herein.

Various techniques, such as production of chimeric or humanized antibodies, may involve procedures of antibody cloning and construction. The antigen-binding V_{L} (variable light chain) and V_{H} (variable heavy chain) sequences for an antibody of interest may be obtained by a variety of molecular cloning procedures, such as (but not limited to) RT-PCR, 5-RACE, and cDNA library screening. The V_{L} and V_{H} genes of an antibody from a cell that expresses a murine antibody can be cloned by PCR amplification and sequenced. To confirm their authenticity, the cloned V_{L} and V_{H} genes can be expressed in cell culture as a chimeric antibody, for example (but not by way of limitation) as described by Orlandi et al. (Proc. Natl. Acad. Sci. USA, 86: 3833 (1989)). Based on the V_{L} and V_{H} gene sequences, a humanized antibody can then be designed and constructed as described by (for example but not by way of limitation) Leung et al. (Mol. Immunol., 32: 1413 (1995)).

The present disclosure further provides nucleic acids encoding any of the human or humanized heavy and light chains described herein. Typically, the nucleic acids also encode a signal peptide fused to the mature heavy and light chains. Coding sequences on nucleic acids can be in operable linkage with regulatory sequences to ensure expression of the coding sequences, such as (but not limited to) a promoter, enhancer, ribosome binding site, transcription termination signal, and the like. The present disclosure in further embodiments includes vectors which comprise the nucleic acids encoding heavy and light chains, and hosts cells which have been transfected with such vectors. The nucleic acids can be synthesized by, for example (but not by way of limitation), solid state synthesis or PCR of overlapping oligonucleotides. Nucleic acids encoding heavy and light chains can be joined as one contiguous nucleic acid, e.g., within an expression vector, or can be separate, e.g., each cloned into its own expression vector.

In certain non-limiting embodiments, this disclosure provides isolated heavy chain polynucleotides encoding a heavy chain variable region of a mAb disclosed herein. This isolated polynucleotide can further encode a human IgG heavy chain constant region (e.g., IgG1, IgG2, IgG3, or IgG4). In one non-limiting embodiment, the amino acid sequence of the IgG constant region comprises one or more substitutions. The disclosure also provides an expression vector comprising said polynucleotide encoding said antibody heavy chain variable region, and further, a host cell comprising that expression vector. In some non-limiting embodiments, the host cell is a mammalian host cell, e.g., a CHO cell.

In one non-limiting embodiment, this disclosure provides an isolated heavy chain polynucleotide encoding a heavy chain variable region of a mAb disclosed herein, and an isolated light chain polynucleotide encoding a light chain variable region of a mAb disclosed herein. The heavy chain polynucleotide can further encode a human IgG heavy chain constant region (e.g., IgG1, IgG2, IgG3, or IgG4). In one non-limiting embodiment, the amino acid sequence of the IgG constant region comprises one or more substitutions. The light chain polynucleotide can further encode a human IgG light chain constant region, e.g., a kappa constant region. The amino acid sequence of the IgG constant region may comprise one or more substitutions. The amino acid sequence of the kappa constant region may comprise one or more substitutions. The disclosure also provides an expression vector comprising said polynucleotide encoding the heavy chain variable region and the light chain variable region. The disclosure also provides a host cell comprising the expression vector. In some non-limiting embodiments, the host cell is a mammalian host cell, e.g., a CHO cell.

In another non-limiting embodiment, this disclosure provides an isolated light chain polynucleotide encoding a light chain variable region of a mAb disclosed herein. This isolated polynucleotide can further encode a human IgG light chain constant region, e.g., a kappa constant region. The amino acid sequence of the kappa constant region may comprise one or more substitutions. The disclosure also provides an expression vector comprising the light chain polynucleotide encoding said light chain variable region, and further, a host cell comprising that expression vector. In some non-limiting embodiments, the host cell is a mammalian host cell, e.g., a CHO cell.

In certain non-limiting embodiments, the present constructs or antigen-binding portions thereof can be synthesized by solid phase procedures well known in the art.

Antibody fragments which recognize specific epitopes can be generated by known techniques. The antibody fragments are antigen binding portions of an antibody, such as (but not limited to) F(ab)₂, Fab′, Fab, Fv, scFv, and other fragments described herein or otherwise contemplated in the art. Other antibody fragments include but are not limited to: the F(ab′)₂ fragments which can be produced by pepsin digestion of the antibody molecule and the Fab′ fragments, which can be generated by reducing disulfide bridges of the F(ab′)₂ fragments. Alternatively, Fab′ expression libraries can be constructed to allow rapid and easy identification of monoclonal Fab′ fragments with the desired specificity. In certain non-limiting embodiments, the antibody fragment may be a fragment that is not an scFv fragment.

An scFv molecule comprises a V_{L} domain and a V_{H} domain. The V_{L} and V_{H} domains associate to form a target binding site. These two domains are further covalently linked by a peptide linker (L). Methods for making scFv molecules and designing suitable peptide linkers are known in the art and include (but are not limited to) those disclosed in U.S. Pat. Nos. 4,704,692 and 4,946,778, for example. An antibody fragment can be prepared by known methods, for example (but not by way of limitation), those disclosed by U.S. Pat. Nos. 4,036,945 and 4,331,647 or others as noted above.

Another form of an antibody fragment is a single-domain antibody (dAb), sometimes referred to as a single chain antibody. Techniques for producing single-domain antibodies are well known in the art (see, for example but not by way of limitation, Cossins et al., Protein Expression and Purification, 51:253-59 (2007); Shuntao et al., Molec. Immunol., 43:1912-19 (2006); and Tanha et al., J. Biol. Chem., 276:24774-780 (2001)).

In certain non-limiting embodiments, the sequences of antibodies, such as (but not limited to) the Fc portions of antibodies, may be varied to optimize the physiological characteristics of the conjugates, such as (but not limited to) the half-life in serum. Methods of substituting amino acid sequences in proteins are widely known in the art, such as (but not limited to) by site-directed mutagenesis (e.g., Green and Sambrook, Molecular Cloning: A laboratory manual, 4^{th} ed., 2014). In certain non-limiting embodiments, the variation may involve the addition or removal of one or more glycosylation sites in the Fc sequence (see, for example but not by way of limitation, U.S. Pat. No. 6,254,868).

The presently disclosed constructs or antigen-binding fragments thereof may have a specific binding K_{D} to a targeted CD protein of less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. Specific binding is detectably higher in magnitude and distinguishable from non-specific binding occurring to at least one unrelated target. For example (but not by way of limitation), the presently disclosed antibodies or antigen-binding fragments thereof have specific binding K_{D} to an epitope comprising of DIR of less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹²M.

As mentioned above, the presently disclosed constructs and fragments thereof can be derivatized or linked to, e.g., conjugated to, therapeutic agents and/or diagnostic agents to form ADCs. For example, an antibody can be functionally linked, directly or indirectly, by covalent bonding or by noncovalent interactions to one or more other molecular entities, such as (but not limited to) another antibody, antibody fragment, a detectable agent, a cytotoxic agent, a pharmaceutical agent, a protein or peptide that can mediate association with another molecule (such as a streptavidin core region or a polyhistidine tag), amino acid linkers, spacers, bridges, signal sequences, immunogenic carriers, or ligands useful in protein purification, such as (but not limited to) glutathione-S-transferase, histidine tag, and staphylococcal protein A. Useful detectable agents with which a protein can be derivatized (or labeled) include (but are not limited to) fluorescent compounds, various enzymes, prosthetic groups, luminescent materials, bioluminescent materials, and radioactive materials. Non-limiting, exemplary fluorescent detectable agents include fluorescein, fluorescein isothiocyanate, rhodamine, and, phycoerythrin. A protein or antibody can also be derivatized with detectable enzymes, such as (but not limited to) alkaline phosphatase, horseradish peroxidase, beta-galactosidase, acetylcholinesterase, glucose oxidase, and the like. A protein can also be derivatized with a prosthetic group (such as, but not limited to, streptavidin/biotin and avidin/biotin).

Particularly suitable (but non-limiting) moieties for conjugation to antibodies are cytotoxic agents (e.g., chemotherapeutic agents), prodrug converting enzymes, radionuclides such as (but not limited to) radioactive isotopes or compounds, an immunomodulator, an anti-angiogenic agent, a pro-apoptotic agent, a cytokine, a chemokine, a drug, a hormone, an siRNA, an enzyme, a growth factor, a prodrug, an oligonucleotide, a pro-apoptotic agent, an interference RNA, a photoactive therapeutic agent, a tyrosine kinase inhibitor, a Bruton kinase inhibitor, a sphingosine inhibitor, a cytotoxic agent, or toxins (these moieties being collectively referred to as therapeutic agents or drugs). Examples of useful classes of cytotoxic agents include (but are not limited to) DNA minor groove binders, DNA alkylating agents, and tubulin inhibitors. Exemplary cytotoxic agents include (but are not limited to) auristatins, camptothecins, duocarmycins, etoposides, maytansines and maytansinoids (e.g., DM1 and DM4), taxanes, benzodiazepines (e.g., pyrrolo[1,4]benzodiazepines (PBDs), indolinobenzodiazepines, and oxazolidinobenzodiazepines), and vinca alkaloids. Techniques for conjugating therapeutic agents to proteins, and in particular to antibodies, are well-known (e.g., see, Carter, PJ and Senter PD, “Antibody-Drug Conjugates for Cancer Therapy.” Cancer J., 14(3):154-169 (2008)).

Examples of radionuclides include, but are not limited to: ¹¹¹In, ¹¹¹At, ¹⁷⁷Lu, ²¹¹Bi, ²¹²Bi, ²¹³Bi, ²¹¹At, ⁶²Cu, ⁶⁷Cu, ⁹⁰Y, ¹²⁵I, ¹³¹I, ¹³³I, ³²P, ³³P, ⁴⁷Sc, ¹¹¹Ag, ⁶⁷Ga, ¹⁵³Sm, ¹⁶¹Tb, ¹⁵²Dy, ¹⁶⁶Dy, ¹⁶¹Ho, ¹⁶⁶Ho, ¹⁸⁶Re, ¹⁸⁸Re, ¹⁸⁹Re, ²¹¹Pb, ²¹²Pb, ²²³Ra, ²²⁵Ac, ²²⁷Th, ⁷⁷As, ⁸⁹Sr, ⁹⁹Mo, ¹⁰⁵Rh ¹⁴⁹Pm, ¹⁶⁹Er, ¹⁹⁴Ir, ⁵⁸Co, ^{80m}Br, ^{99m}Tc, ^{103m}Rh, ¹⁰⁹Pt, ¹¹⁹Sb, ¹²⁵I, ^{189m}Os, ¹⁹²Ir, ²¹⁹Rn, ²¹⁵Po, ²²¹Fr, ²⁵⁵Fm, ¹¹C, ¹³N, ¹⁵O, ⁷⁵Br, ¹⁹⁸Au, ¹⁹⁹Au, ²²⁴Ac, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ²²⁷Th, ^{125m}Te, ¹⁶⁵Tm, ¹⁶⁷Tm, ¹⁶⁸Tm, ¹⁹⁷Pt, ¹⁰⁹Pd, ¹⁴²Pr, ¹⁴³Pr, ¹⁶¹Tb, ⁵⁷Co, ⁵⁸Co, ⁵¹Cr, ⁵⁹Fe, ⁷⁵Se, ²⁰¹Tl, ⁷⁶Br, and ¹⁶⁹Yb.

Examples of diagnostic agents include, but are not limited to: radionuclide, a contrast agent, a fluorescent agent, a chemiluminescent agent, a bioluminescent agent, a paramagnetic ion, an enzyme, and a photoactive diagnostic agent.

Examples of diagnostic radionuclides include, but are not limited to: ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²F, ¹¹C, ¹³N, ¹⁵O, ¹⁸⁶Re, ¹⁸⁸Re, ⁵¹Mn, ^{52m}Mn, ⁵⁵Co, ⁷²As, ⁷⁵Br, ⁷⁶Br, ^{82m}Rb, ⁸³Sr, or other gamma-, beta-, or positron-emitters.

Examples of paramagnetic ions include, but are not limited to: chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), and erbium (III).

Examples of fluorescent labeling diagnostic agents include, but are not limited to: fluorescein isothiocyanate, rhodamine, phycoerytherin, phycocyanin, allophycocyanin, o-phthaldehyde, and fluorescamine; or a chemiluminescent labeling compound selected from the group comprising luminol, isoluminol, an aromatic acridinium ester, an imidazole, an acridinium salt, and an oxalate ester; or a bioluminescent compound selected from the group comprising luciferin, luciferase, and aequorin.

As noted, in certain non-limiting embodiments, the constructs or fragments thereof may be used in combination with one or more therapeutic and/or diagnostic agents. Where the agent is attached to a construct or fragment thereof to be administered by subcutaneous, intramuscular, or transdermal administration, then only non-cytotoxic agents are contemplated. Non-cytotoxic agents may include, without limitation, immunomodulators, cytokines (and their inhibitors), chemokines (and their inhibitors), tyrosine kinase inhibitors, growth factors, hormones and certain enzymes (i.e., those that do not induce local necrosis), or their inhibitors. Where the agent is co-administered either before, simultaneously with, or after the subcutaneous, intramuscular, or transdermal multispecific construct formulation, then cytotoxic agents may be utilized. An agent may be administered as an immunoconjugate with a second antibody or fragment thereof, or may be administered as a free agent. The following discussion applies to both cytotoxic and non-cytotoxic agents.

Examples of therapeutic agents that can be conjugated (or delivered separately) include, but are not limited to: 5-fluorouracil, aplidin, azaribine, anastrozole, anthracyclines, bendamustine, bleomycin, bortezomib, bryostatin-1, busulfan, calicheamycin, camptothecin, carboplatin, 10-hydroxycamptothecin, carmustine, celecoxib, chlorambucil, cisplatinum, Cox-2 inhibitors, CPT-11 SN-38, carboplatin, cladribine, camptothecans, cyclophosphamide, cytarabine, dacarbazine, docetaxel, dactinomycin, daunorubicin, doxorubicin, 2-pyrrolinodoxorubicine (2P-DOX), pro-2P-DOX, cyano-morpholino doxorubicin, doxorubicin glucuronide, epirubicin glucuronide, estramustine, epipodophyllotoxin, estrogen receptor binding agents, etoposide (VP16), etoposide glucuronide, etoposide phosphate, floxuridine (FUdR), 3′,5′-O-dioleoyl-FudR (FUdR-dO), fludarabine, flutamide, farnesyl-protein transferase inhibitors, gemcitabine, hydroxyurea, idarubicin, ifosfamide, L-asparaginase, lenolidamide, leucovorin, lomustine, mechlorethamine, melphalan, mercaptopurine, 6-mercaptopurine, methotrexate, mitoxantrone, mithramycin, mitomycin, mitotane, navelbine, nitrosourea, plicomycin, procarbazine, paclitaxel, pentostatin, PSI-341, raloxifene, semustine, streptozocin, tamoxifen, paclitaxel, temazolomide, transplatinum, thalidomide, thioguanine, thiotepa, teniposide, topotecan, uracil mustard, vinorelbine, vinblastine, vincristine, a vinca alkaloid, a tyrophostin, canertinib, dasatinib, erlotinib, gefitinib, imatinib, lapatinib, leflunomide, nilotinib, pazopanib, semaxinib, sorafenib, sunitinib, sutent, vatalanib, PCI-32765 (ibrutinib), PCI-45292, GDC-0834, LFM-A13, and RN486.

Examples of toxins include, but are not limited to: ricin, abrin, alpha toxin, saporin, ribonuclease (RNase; e.g., onconase), DNase I, Staphylococcal enterotoxin-A, pokeweed antiviral protein, gelonin, diphtheria toxin, Pseudomonas exotoxin, and Pseudomonas endotoxin.

Immunomodulators include, but are not limited to: cytokines, stem cell growth factors, lymphotoxins, hematopoietic factors, colony stimulating factors (CSF), interferons (IFN), erythropoietins, thrombopoietins, and combinations thereof. Specifically useful are lymphotoxins such as (but not limited to) tumor necrosis factor (TNF); hematopoietic factors such as (but not limited to) interleukin (IL); colony stimulating factors such as (but not limited to) granulocyte-colony stimulating factor (G-CSF) or granulocyte macrophage-colony stimulating factor (GM-CSF); interferons such as (but not limited to) interferons-alpha, -beta, -lambda, or -gamma; and stem cell growth factors such as (but not limited to) that designated “S1 factor.” Included among the cytokines are growth hormones such as (but not limited to): human growth hormone, N-methionyl human growth hormone, and bovine growth hormone; parathyroid hormone; thyroxine; insulin; proinsulin; relaxin; prorelaxin; glycoprotein hormones such as (but not limited to) follicle stimulating hormone (FSH), thyroid stimulating hormone (TSH), and luteinizing hormone (LH); hepatic growth factor; prostaglandin; fibroblast growth factor; prolactin; placental lactogen; OB protein; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as (but not limited to) NGF-beta; platelet-growth factor; transforming growth factors (TGFs) such as (but not limited to) TGF-alpha and TGF-beta; insulin-like growth factor-I and -II; erythropoietin (EPO); osteoinductive factors; interferons such as (but not limited to) interferon-alpha, -beta, -lambda, and -gamma; colony stimulating factors (CSFs) such as (but not limited to) macrophage-CSF (M-CSF); interleukins (ILs) such as (but not limited to) IL-1, IL-1 alpha, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-23, IL-25; leukemia inhibitory factor (LIF); kit-ligand or FLT-3 ligand; angiostatin; thrombospondin; endostatin; tumor necrosis factor; and lymphotoxin. Chemokines of use include (but are not limited to): RANTES, MCAF, MIP1-alpha, MIP1-Beta, and IP-10.

In certain non-limiting embodiments, therapeutic radionuclides have a decay-energy in the range of 20 to 6,000 keV, such as (but not limited to) in the ranges of: 60 to 200 keV for an Auger emitter; 100-2,500 keV for a beta emitter; and 4,000-6,000 keV for an alpha emitter. Maximum decay energies of useful beta-particle-emitting nuclides may be, but are not limited to, 20-5,000 keV; 100-4,000 keV; or 500-2,500 keV. Also included are radionuclides that substantially decay with Auger-emitting particles, such as (but not limited to): Co-58, Ga-67, Br-80m, Tc-99m, Rh-103m, Pt-109, In-111, Sb-119, 1-125, Ho-161, Os-189m, and Ir-192. Decay energies of useful beta-particle-emitting nuclides may be (for example but not by way of limitation): <1,000 keV, <100 keV, or <70 keV. Also included are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213, Th-227, and Fm-255. Decay energies of useful alpha-particle-emitting radionuclides include (but are not limited to): 2,000-10,000 keV; 3,000-8,000 keV; or 4,000-7,000 keV.

A connection between the construct (or fragment thereof) and the linker can be via a number of different routes, such as (but not limited to): through a thioether bond, through a disulfide bond, through an amide bond, or through an ester bond. In one non-limiting embodiment, the connection between the construct and the linker is formed between a thiol group of a cysteine residue of the construct and a maleimide group of the linker. In some non-limiting embodiments, the interchain bonds of the construct are converted to free thiol groups prior to reaction with the functional group of the linker. In some non-limiting embodiments, a cysteine residue is introduced into a heavy or light chain of a construct and reacted with the linker. Positions for cysteine insertion by substitution in heavy or light chains include (but are not limited to) those described in U.S. Patent Application Publication No. 2007/0092940 and International Patent Application Publication No. WO 2008/070593.

In some non-limiting embodiments, the ADCs have the formula: Mab-(LU-D)ₙ, wherein Mab is a CD-targeted construct, or fragment thereof, LU is a Linker unit, and D is a Drug unit (i.e., the therapeutic or diagnostic agent). The subscript n ranges for example (but not by way of limitation) from 1 to 20 (i.e., 1, 2, 3, 4, 5, 6, 7, 8, 9, 10, 11, 12, 13, 14, 15, 16, 17, 18, 19, or 20), or more. Such conjugates comprise the construct covalently linked to at least one drug via a linker. The LU may be connected at one end to the construct and at the other end to a drug molecule. The skilled artisan will appreciate that in some aspects, the subscript n represents the number of drug-linkers on a singular construct. In other aspects, n represents the average number of drug-linker molecules per construct, e.g., the average number of drug-linkers per antibody in a reaction mixture or composition (e.g., pharmaceutical composition), and can be an integer or non-integer value. Accordingly, in some aspects, for compositions (e.g., pharmaceutical compositions), n represents the average drug loading of the ADCs in the composition, and n ranges from 1 to 20. In some non-limiting embodiments, the present disclosure provides construct -linker conjugates have the formula: Mab-(LU)ₙ, wherein Mab is a CD-targeted construct or fragment thereof, and LU is a Linker unit for linking a drug to the construct. The subscript n ranges for example from 1 to 20 (i.e., 1, 2, 3, 4, 5, 6, 7, 8, 9, 10, 11, 12, 13, 14, 15, 16, 17, 18, 19, or 20), or more. Such conjugates comprise an anti-CD construct covalently linked to one or more linkers, wherein the Linker Unit is connected at one end to the construct and has a free end for connecting to a drug molecule. In some non-limiting embodiments, n is from about 1 to about 18 drugs per construct. In some non-limiting embodiments, n is 1. In some non-limiting embodiments, n is 2. In some non-limiting embodiments, n is from about 2 to about 12 drugs per construct. In some non-limiting embodiments, n is in a range of from about 2 to about 10, a range of from about 2 to about 8, a range of from about 2 to about 6, a range of from about 2 to about 5, a range of from about 2 to about 4, or a range of from about 2 to about 3 per construct.

In certain non-limiting embodiments, the therapeutic and/or diagnostic agent may be covalently attached to a construct or fragment thereof to form an immunoconjugate. In some non-limiting embodiments, the therapeutic and/or diagnostic agent may be attached to an antibody or fragment thereof via a carrier moiety. Carrier moieties may be attached, for example (but not by way of limitation) to reduced SH groups and/or to carbohydrate side chains. A carrier moiety can be attached at the hinge region of a reduced antibody component via disulfide bond formation. Alternatively, such agents can be attached using a heterobifunctional cross-linker, such as (but not limited to) N-succinyl 3-(2-pyridyldithio)propionate (e.g., see Yu et al., Int. J. Cancer, 56: 244 (1994). General techniques for such conjugation are well-known in the art; see, for example, Wong, Chemistry Of Protein Conjugation And Cross-Linking (CRC Press, 1991); Upeslacis et al., “Modification of Antibodies by Chemical Methods,” in Monoclonal Antibodies: Principles And Applications, Birch et al. (eds.), pages 187-230 (Wiley-Liss, Inc., 1995); and Price, “Production and Characterization of Synthetic Peptide-Derived Antibodies,” in Monoclonal Antibodies: Production, Engineering And Clinical Application, Ritter et al. (eds.), pages 60-84 (Cambridge University Press, 1995). Alternatively, the carrier moiety can be conjugated via a carbohydrate moiety in the Fc region of the construct.

Methods for conjugating functional groups to antibodies via an antibody carbohydrate moiety are well-known to those of skill in the art; see, for example, Shih et al., Int. J. Cancer, 41: 832 (1988); Shih et al., Int. J. Cancer, 46: 1101 (1990); and U.S. Pat. No. 5,057,313. The general method involves reacting an antibody having an oxidized carbohydrate portion with a carrier polymer that has at least one free amine function. This reaction results in an initial Schiff base (imine) linkage, which can be stabilized by reduction to a secondary amine to form the final conjugate.

Other methods of chemical conjugation of such moieties to biomolecules are well known in the art, and any such known method may be utilized to form an antibody conjugate that functions in accordance with the present disclosure. Such methods of immunoconjugate formation are disclosed, for example (but not by way of limitation), in U.S. Pat. Nos. 4,699,784; 4,824,659; 5,525,338; 5,677,427; 5,697,902; 5,716,595; 6,071,490; 6,187,284; 6,306,393; 6,548,275; 6,653,104; 6,962,702; 7,033,572; 7,147,856; and 7,259,240.

Any of the multispecific constructs described herein can be used in pharmaceutical compositions and formulations. Pharmaceutical compositions and formulations of the construct and/or other agents describe herein can be prepared by mixing one, two, or all three agents having the desired degree of purity with one or more optional pharmaceutically acceptable carriers. Non-limiting examples of such pharmaceutically acceptable carriers are shown in Remington: The Science and Practice of Pharmacy, 23^{rd} Ed. (2020). The pharmaceutically acceptable carriers may be provided in the form of lyophilized formulations or aqueous solutions. Pharmaceutically-acceptable carriers are generally nontoxic to recipients at the dosages and concentrations employed, and include, but are not limited to: buffers such as phosphate, citrate, and other organic acids; antioxidants including ascorbic acid, glutathione, and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (e.g., less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, sucrose, dextrins, and dextrans; chelating agents such as EDTA; sugars such as sucrose, mannitol, trehalose or sorbitol; salt-forming counterions such as sodium; metal complexes (e.g., Zn-protein complexes); detergents and/or non-ionic surfactants such as polyethylene glycol (PEG). Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer. Other physiologically acceptable compounds include (but are not limited to) wetting agents, emulsifying agents, dispersing agents, or preservatives.

The multispecific constructs may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington, 2020, op. cit. Sustained-release preparations may be prepared. Suitable examples of sustained-release preparations include semipermeable matrices of solid hydrophobic polymers containing the construct, which matrices are in the form of shaped articles, for example, films, or microcapsules.

The present constructs or antigen-binding portions thereof can be formulated into compositions for delivery to a mammalian subject. The composition can be administered alone and/or mixed with a pharmaceutically acceptable vehicle or excipient. Suitable vehicles are, for example (but not by way of limitation), water, saline, dextrose, glycerol, ethanol, or the like, and combinations thereof. In addition, the vehicle can contain minor amounts of auxiliary substances such as (but not limited to) wetting or emulsifying agents, pH buffering agents, or adjuvants. The compositions of the present disclosure can also include ancillary substances, such as (but not limited to) pharmacological agents, cytokines, or other biological response modifiers.

Furthermore, the compositions can be formulated into compositions in either neutral or salt forms. Pharmaceutically acceptable salts include (but are not limited to) the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, and procaine.

Compositions can be administered in a single dose treatment or in multiple dose treatments on a schedule and over a time period appropriate to the age, weight, and condition of the subject, the particular composition used, and the route of administration. In one non-limiting embodiment, a single dose of the composition according to the disclosure is administered. In other non-limiting embodiments, multiple doses are administered. The frequency of administration can vary depending on any of a variety of factors, e.g., severity of the symptoms, degree of immunoprotection desired, or whether the composition is used for prophylactic or curative purposes. For example, in certain non-limiting embodiments, the composition is administered once per month, twice per month, three times per month, every other week, once per week, twice per week, three times per week, four times per week, five times per week, six times per week, every other day, daily, twice a day, or three times a day. The duration of treatment (i.e., the period of time over which the composition is administered) can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

The dosage of an administered active agent for humans will vary depending upon factors such as (but not limited to) the patient's age, weight, height, sex, general medical condition, and previous medical history. In certain non-limiting embodiments, the recipient is provided with a dosage of the active agent that is in the range of from about 1 mg to about 1000 mg as a single infusion or single or multiple injections, although a lower or higher dosage also may be administered. In certain non-limiting embodiments, the dosage may be in the range of from about 25 mg to about 100 mg of the active agent per square meter (m²) of body surface area for a typical adult, although a lower or higher dosage also may be administered. Non-limiting examples of dosages of the active agent that may be administered to a human subject further include 1 to 500 mg, 1 to 70 mg, or 1 to 20 mg, although higher or lower doses may be used. Dosages may be repeated as needed, for example (but not by way of limitation), once per week for 4-10 weeks, once per week for 8 weeks, or once per week for 4 weeks. It may also be given less frequently, such as (but not limited to) every other week for several months, or more frequently, such as twice weekly or by continuous infusion.

In at least one embodiment, the present disclosure is directed to a dosing regimen involving administration of the multispecific construct such as disclosed elsewhere herein. The dosing regimen may comprise multiple dosing cycles (e.g., wherein the first dosing cycle is a step-up, fractionated dosing cycle). The doses may range from about 0.02 mg to about 2.0 mg (e.g., from about 0.02 to about 1.8 mg, from about 0.02 to about 1.6 mg, from about 0.02 to about 1.4 mg, from about 0.02 to about 1.2 mg, from about 0.05 to about 1.8 mg, from about 0.1 to about 1.8 mg, from about 0.4 to about 1.8 mg, from about 0.6 to about 1.8 mg, from about 0.8 to about 1.8 mg, from about 0.5 to about 1.5 mg, from about 0.8 to about 1.2 mg; e.g., about 1 mg), from about 0.05 mg to about 4.0 mg (e.g., from about 0.05 to about 3.5 mg, from about 0.05 to about 3.0 mg, from about 0.05 to about 2.5 mg, from about 0.05 to about 2.2 mg, from about 0.1 to about 3.5 mg, from about 0.5 to about 3.5 mg, from about 1.0 to about 3.5 mg, from about 1.5 to about 3.5 mg, from about 1.8 to about 3.5 mg, from about 1.0 to about 3.0 mg, from about 1.5 to about 2.5 mg; e.g., about 2 mg).

In some embodiments, the dose may range from 50 mg to 200 mg (e.g., from 50 mg to 175 mg, from 50 mg to 150 mg, from 50 mg to 125 mg, from 50 mg to 100 mg, from 50 mg to 75 mg, from 50 mg to 70 mg, from 52 mg to 100 mg, from 52 mg to 75 mg, from 50 mg to 180 mg, from 55 mg to 150 mg, from 55 mg to 100 mg, from 55 mg to 70 mg, from 55 mg to 65 mg, from 58 mg to 62 mg; e.g., about 60 mg). In some embodiments, the dose may be about 60 mg. In some embodiments, the dose is about 1 mg. In some embodiments, the dose is about 2 mg.

In some embodiments, the dose is from 20 mg to 200 mg (e.g., from 20 mg to 175 mg, from 20 mg to 150 mg, from 20 mg to 100 mg, from 20 mg to 75 mg, from 30 mg to 175 mg, from 40 mg to 175 mg, from 45 mg to 175 mg, from 50 mg to 175 mg, from 30 mg to 150 mg, from 40 mg to 100 mg, from 45 mg to 75 mg, from 50 mg to 70 mg, from 55 mg to 65 mg, from 58 mg to 62 mg; about 20 mg, about 30 mg, about 45 mg, or e.g., about 60 mg). In some embodiments, the dose is from about 12 mg to about 48 mg (e.g., from about 12 mg to about 42 mg, from about 12 mg to about 36 mg, from about 12 mg to about 30 mg, from about 18 mg to about 48 mg, from about 18 mg to about 42 mg, from about 24 mg to about 42 mg, from about 27 mg to about 42 mg, from about 24 mg to about 36 mg, from about 27 mg to about 33 mg, from about 28 mg to about 32 mg; e.g., about 24 mg, about 27 mg, about 30 mg, about 33 mg, or about 36 mg).

In some embodiments, the dosing regimen comprises administration of a loading dose, such as from 20 mg to 200 mg (e.g., from 20 mg to 175 mg, from 20 mg to 150 mg, from 20 mg to 100 mg, from 20 mg to 75 mg, from 30 mg to 175 mg, from 40 mg to 175 mg, from 45 mg to 175 mg, from 50 mg to 175 mg, from 30 mg to 150 mg, from 40 mg to 100 mg, from 45 mg to 75 mg, from 50 mg to 70 mg, from 55 mg to 65 mg, from 58 mg to 62 mg; e.g., about 60 mg). In some embodiments, the dose is from about 12 mg to about 48 mg (e.g., from about 12 mg to about 42 mg, from about 12 mg to about 36 mg, from about 12 mg to about 30 mg, from about 18 mg to about 48 mg, from about 18 mg to about 42 mg, from about 24 mg to about 42 mg, from about 27 mg to about 42 mg, from about 24 mg to about 36 mg, from about 27 mg to about 33 mg, from about 28 mg to about 32 mg; e.g., about 24 mg, about 27 mg, about 30 mg, about 33 mg, or about 36 mg).

In some non-limiting embodiments, the active agent is provided in a concentration of about 1 nM, about 5 nM, about 10 nM, about 25 nM, about 50 nM, about 75 nM, about 100 nM, about 150 nM, about 200 nM, about 250 nM, about 300 nM, about 350 nM, about 400 nM, about 500 nM, about 550 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 60 μM, about 70 μM, about 75 μM, about 80 μM, about 90 μM, about 100 μM, about 125 μM, about 150 μM, about 175 μM, about 200 μM, about 250 μM, about 300 μM, about 350 μM, about 400 μM, about 500 μM, about 600 μM, about 700 μM, about 750 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 400 mM, about 500 mM, about 600 mM, about 700 mM, about 800 mM, about 900 mM, about 1000 mM, about 1 M, about 1.1 M, about 1.2 M, about 1.3 M, about 1.4 M, about 1.5 M, about 1.6 M, about 1.7 M, about 1.8 M, about 1.9 M, about 2 M, about 3 M, about 4 M, about 5 M, about 6 M, about 7 M, about 8 M, about 9 M, about 10 M, about 15 M, about 20 M, about 25 M, about 30 M, about 35 M, about 40 M, about 45 M, about 50 M, about 75 M, about 100 M, or any range in between any two of the aforementioned concentrations, including said two concentrations as endpoints of the range, or any number in between any two of the aforementioned concentrations.

When administered orally, the present compositions may be protected from digestion. This can be accomplished either by complexing the construct or antigen-binding portion thereof with a composition to render it resistant to acidic and enzymatic hydrolysis or by packaging the construct or antigen-binding portion thereof in an appropriately resistant carrier such as (but not limited to) a liposome, e.g., such as shown in U.S. Pat. No. 5,391,377.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical transdermal administration, the agents are formulated into ointments, creams, salves, powders, and gels. Transdermal delivery systems can also include (for example but not by way of limitation) patches. The present compositions can also be administered in sustained delivery or sustained release mechanisms. For example, biodegradeable microspheres or capsules or other biodegradeable polymer configurations capable of sustained delivery of a peptide can be included herein.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include (for example but not by way of limitation) air jet nebulizers.

In one aspect, the pharmaceutical formulations comprising compositions or nucleic acids, antibodies or fragments thereof are incorporated in lipid monolayers or bilayers, such as (but not limited to) liposomes, such as shown in U.S. Pat. Nos. 6,110,490; 6,096,716; 5,283,185; and 5,279,833. In other aspects, non-limiting embodiments of the disclosure include formulations in which the polypeptides or nucleic acids have been attached to the surface of the monolayer or bilayer of the liposomes. Liposomes and liposomal formulations can be prepared according to standard methods and are also well known in the art, such as (but not limited to) those disclosed in U.S. Pat. Nos. 4,235,871; 4,501,728; and 4,837,028.

In one aspect, the compositions are prepared with carriers that will protect the construct or fragment thereof against rapid elimination from the body, such as (but not limited to) a controlled release formulation, including implants and microencapsulated delivery systems. Biodegradable, biocompatible polymers can be used, such as (but not limited to) ethylene vinyl acetate, polyanhydrides, polyglycolic acid, collagen, polyorthoesters, and polylactic acid. Methods for preparation of such formulations will be apparent to those skilled in the art.

The constructs and fragments thereof in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active compound is combined in a mixture with one or more pharmaceutically suitable excipients. Sterile phosphate-buffered saline is one non-limiting example of a pharmaceutically suitable excipient.

Non-limiting examples of routes of administration of the compositions described herein include parenteral injection, e.g., by subcutaneous, intramuscular, or transdermal delivery. Other forms of parenteral administration include (but are not limited to) intravenous, intraarterial, intralymphatic, intrathecal, intraocular, intracerebral, or intracavitary injection. In parenteral administration, the compositions will be formulated in a unit dosage injectable form such as (but not limited to) a solution, suspension, or emulsion, in association with a pharmaceutically acceptable excipient. Such excipients are inherently nontoxic and nontherapeutic. Non-limiting examples of such excipients include saline, Ringer's solution, dextrose solution, and Hanks' solution. Nonaqueous excipients such as (but not limited to) fixed oils and ethyl oleate may also be used. An alternative non-limiting excipient is 5% dextrose in saline. The excipient may contain minor amounts of additives such as (but not limited to) substances that enhance isotonicity and chemical stability, including buffers and preservatives. The constructs can be delivered or administered alone or as pharmaceutical compositions by any means known in the art, such as (but not limited to) systemically, regionally, or locally; by intra-arterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa).

Administration can be (for example but not by way of limitation) parenteral, intravenous, oral, subcutaneous, intra-arterial, intracranial, intrathecal, intraperitoneal, topical, intranasal, or intramuscular. Administration can also be localized directly into a tumor. Administration into the systemic circulation by intravenous or subcutaneous administration is typical. Intravenous administration can be, for example (but not by way of limitation), by infusion over a period such as (but not limited to) 30-90 min or by a single bolus injection.

Formulated compositions comprising the constructs can be used (for example but not by way of limitation) for subcutaneous, intramuscular, or transdermal administration. Compositions can be presented in unit dosage form, e.g., in ampoules or in multi-dose containers, with an added preservative. Compositions can also take such forms as suspensions, solutions, or emulsions in oily or aqueous vehicles, and can contain formulatory agents such as suspending, stabilizing, and/or dispersing agents.

The compositions may be administered in solution. The formulation thereof may be in a solution having a suitable pharmaceutically acceptable buffer, such as (but not limited to) phosphate, Tris (hydroxymethyl) aminomethane-HCl, or citrate, and the like. Buffer concentrations should be in the range of 1 to 100 mM. The formulated solution may also contain a salt, such as (but not limited to) sodium chloride or potassium chloride in a concentration of 50 to 150 mM. An effective amount of a stabilizing agent such as (but not limited to) mannitol, trehalose, sorbitol, glycerol, albumin, a globulin, a detergent, a gelatin, a protamine, or a salt of protamine may also be included.

Exemplary, non-limiting ranges for a therapeutically or prophylactically effective amount of a construct or mAb-derived compound of the present disclosure antibody-drug conjugate include a range of from about 0.001 mg/kg of the subject's body weight to about 100 mg/kg of the subject's body weight, such as but not limited to a range of from about 0.01 mg/kg to about 50 mg/kg, a range of from about 0.1 mg/kg to about 50 mg/kg, a range of from about 0.1 mg/kg to about 40 mg/kg, a range of from about 1 mg/kg to about 30 mg/kg, a range of from about 1 mg/kg to about 20 mg/kg, a range of from about 2 mg/kg to about 30 mg/kg, a range of from about 2 mg/kg to about 20 mg/kg, a range of from about 2 mg/kg to about 15 mg/kg, a range of from about 2 mg/kg to about 12 mg/kg, a range of from about 2 mg/kg to about 10 mg/kg, a range of from about 3 mg/kg to about 30 mg/kg, a range of from about 3 mg/kg to about 20 mg/kg, a range of from about 3 mg/kg to about 15 mg/kg, a range of from about 3 mg/kg to about 12 mg/kg, or a range of from about 3 mg/kg to about 10 mg/kg, or a range of from about 10 mg to about 1500 mg as a fixed dosage.

In some non-limiting methods, the patient is administered the construct or fragment thereof every one, two, three, or four weeks, for example. The dosage depends on the frequency of administration, condition of the patient, response to prior treatment (if any), whether the treatment is prophylactic or therapeutic, and whether the disorder is acute or chronic, among other factors.

The frequency of administration depends on the half-life of the construct or fragment thereof in the circulation, the condition of the patient, and the route of administration, among other factors. The frequency can be daily, weekly, monthly, quarterly, or at irregular intervals in response to changes in the patient's condition or progression of the disorder treated. An exemplary (but non-limiting) frequency for intravenous administration is between twice a week and quarterly over a continuous course of treatment, although more or less frequent dosing is also possible. Other exemplary (but non-limiting) frequencies for intravenous administration are between once weekly or once monthly over a continuous course of treatment, although more or less frequent dosing is also possible. For subcutaneous administration, an exemplary (but non-limiting) dosing frequency is daily to monthly, although more or less frequent dosing is also possible.

The number of dosages administered may depends on the severity and temporal nature of the disorder (e.g., whether presenting acute or chronic symptoms) and the response of the disorder to the treatment. For acute disorders or acute exacerbations of a chronic disorder, between 1 and 10 doses may be used. Sometimes a single bolus dose, optionally in divided form, is sufficient for an acute disorder or acute exacerbation of a chronic disorder. Treatment can be repeated for recurrence of an acute disorder or acute exacerbation. For chronic disorders, the active agent may be administered at regular intervals, such as (but not limited to) weekly, fortnightly, monthly, quarterly, every six months for at least 1, 5, or 10 years, or for the life of the patient.

In some non-limiting embodiments, the construct or fragment thereof comprises a sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the sequence of the variable heavy chain and/or variable light chain sequences of the mAbs or antibody fragments disclosed herein. In some non-limiting embodiments the active agent comprises a sequence that is 100% identical to the variable heavy chain and variable light chain sequences of the presently-disclosed mAbs over a span of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, or 118 residues.

In certain non-limiting embodiments, the percent identity of two amino acid sequences (or two nucleic acid sequences) can be determined, for example, by aligning the sequences for optimal comparison purposes (e.g., gaps can be introduced in the sequence of a first sequence). The amino acids or nucleotides at corresponding positions are then compared, and the percent identity between the two sequences is a function of the number of identical positions shared by the sequences (i.e., % identity=(no. of identical positions÷total no. of positions)×100). The actual comparison of the two sequences can be accomplished by well-known methods, for example, using a mathematical algorithm. A specific, non-limiting example of such a mathematical algorithm is described in Karlin et al. (Proc. Natl. Acad. Sci. USA, 90:5873-5877 (1993)). Such an algorithm is incorporated into the BLASTN and BLASTX programs (version 2.2) as described in Schaffer et al. (Nucleic Acids Res., 29:2994-3005 (2001)). When utilizing BLAST and Gapped BLAST programs, the default parameters of the respective programs (e.g., BLASTN) can be used. In one non-limiting embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Several non-limiting embodiments also encompass variants of the constructs or fragments thereof disclosed herein, comprising one or more amino acid residue substitutions in the V_{L} domain and/or V_{H} domain thereof. Several non-limiting embodiments also encompass variants of the above described antibodies with one or more additional amino acid residue substitutions in one or more V_{L} CDRs and/or one or more V_{H} CDRs. The variants generated by introducing substitutions in the V_{H} domain, V_{H} CDRs, V_{L} domain, and/or V_{L} CDRs described herein can be tested in vitro and in vivo, for example, for its ability to bind to a disclosed CD protein (by, e.g., immunoassays including, but not limited to, ELISAs and BIAcore).

Some non-limiting embodiments of the methods provided herein include identifying a subject for treatment with a construct or fragment thereof active agent disclosed herein. For example, one method includes determining if the subject has an elevated level of autoantibodies, as compared to the level of the same autoantibodies in a serum sample of normal subject. Some non-limiting embodiments include selecting a subject for treatment with the active agents of the present disclosure if the serum sample has an elevated level of autoantibodies.

Some non-limiting embodiments provided herein include kits. In some non-limiting embodiments, a kit can include any of the construct or fragment thereof as disclosed or otherwise described which is able to bind to an AAB-producing B cell. In some non-limiting embodiments, the construct or binding fragment thereof is lyophilized. In some non-limiting embodiments, the construct or binding fragment thereof is in aqueous solution, or other carrier as described herein. In some non-limiting embodiments, the kit includes a pharmaceutical carrier for administration of the construct or binding fragment thereof. In some non-limiting embodiments, the kit also includes a chemotherapeutic agent. Certain non-limiting embodiments of the present disclosure include kits containing components suitable for treatments or diagnosis. In one example, the kit contains at least one construct or binding fragment thereof which is able to bind to CD68 and CD20, or any other combination of CD68 and a B cell surface marker protein. A device capable of delivering the kit components by injection, for example, a syringe for subcutaneous injection, may be included in some non-limiting embodiments. Where transdermal administration is used, a delivery device such as hollow microneedle delivery device may be included in the kit in some non-limiting embodiments. Exemplary transdermal delivery devices are known in the art, such as (but not limited to) a hollow Microstructured Transdermal System (e.g., 3M Corp.), and any such known device may be used. The kit components may be packaged together or separated into two or more containers. In some non-limiting embodiments, the containers may be vials that contain sterile, lyophilized formulations of a composition that are suitable for reconstitution. A kit may also contain one or more buffers suitable for reconstitution and/or dilution of other reagents. Alternatively, the construct or binding fragment may be delivered and stored as a liquid formulation. Other containers that may be used include, but are not limited to, a pouch, tray, box, tube, or the like. Kit components may be packaged and maintained sterilely within the containers. Another component that can be included is instructions for the use of the kit for treatment of certain diseases or conditions or for the diagnosis of such.

The kit or article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be a vial having a stopper pierceable by a hypodermic injection needle). At least one active agent in the composition is a multispecific construct as described herein. The label or package insert indicates that the composition is used for treating the condition of choice (e.g., SLE) and further includes dosing information, for example one of the dosing regimens described herein. Moreover, the kit or article of manufacture may comprise (a) a first container with a composition contained therein, wherein the composition comprises a multispecific construct as described herein. The kit may further include other materials desirable from a commercial and user standpoint, including other buffers, diluents, filters, needles, and syringes. The term “package insert” is used to refer to instructions customarily included in commercial packages of therapeutic products, that contain information about the indications, usage, dosage, administration, combination therapy, contraindications and/or warnings concerning the use of such therapeutic products.

In various embodiments of the present disclosure, the therapeutic multispecific constructs described herein (e.g., bispecific antibodies) can be used as treatments for autoimmune disorders and diseases, including but not limited to, Systemic lupus erythematosus (SLE), SLE flare-ups, lupus nephritis (LN), acute Graft versus Host Disease (GvHD), chronic GvHD, Rheumatoid Arthritis (RA), Pemphigus, e.g., Pemphigus vulgaris (PVu), Pemphigus vegetans (PVe), Pemphigus erythematosus (PE), and Pemphigus foliaceus (PF), Chronic Lymphocytic Leukemia (CLL), Granulomatosis with Polyangiitis (GPA), Microscopic Polyangiitis (MPA), Multiple Sclerosis (MS), Primary Progressive Multiple Sclerosis (PPMS), Relapsing Multiple Sclerosis (RMS), Anti-neutrophil Cytoplasmic Antibody (ANCA)-associated vasculitis, Non-Hodgkin's Lymphoma (NHL), Rassmussen's encephalitis, Immune Thrombocytopenic Purpura (ITP), Follicular Lymphoma Leukemia (FLL), and other immune conditions treatable by B cell depletion therapies (BCDT), Inflammatory Bowel Disease (IBD), Ulcerative colitis (UC), Crohn's disease (CD), Pediatric Acute-onset Neuropsychiatric Syndrome (PANS), and Pediatric Autoimmune Neuropsychiatric Disorder Associated with Streptoccocal Infections (PANDAS) (See Table 1).

TABLE 1
Autoimmune conditions and Diseases

Systemic lupus erythematosus (SLE),
SLE flare-ups,
lupus nephritis (LN),
acute Graft versus Host Disease (GvHD),
chronic GvHD,
Rheumatoid Arthritis (RA),
Pemphigus,
Pemphigus vulgaris (PVu),
Pemphigus vegetans (PVe),
Pemphigus erythematosus (PE),
Pemphigus foliaceus (PF),
Chronic Lymphocytic Leukemia (CLL),
Granulomatosis with Polyangiitis (GPA),
Microscopic Polyangiitis (MPA),
Multiple Sclerosis (MS),
Primary Progressive Multiple Sclerosis (PPMS),
Relapsing Multiple Sclerosis (RMS),
Anti-neutrophil Cytoplasmic Antibody (ANCA)-associated vasculitis,
Non-Hodgkin's Lymphoma (NHL),
Rassmussen's encephalitis,
Immune Thrombocytopeniarpura (ITP),
Follicular Lymphoma Leukemia (FLL),
immune conditions treatable by B cell depletion therapies (BCDT),
Inflammatory Bowel Diseases (IBD),
Ulcerative colitis (UC),
Crohn's disease CD),
Pediatric Acute-onset Neuropsychiatric Syndrome (PANS),
Pediatric Autoimmune Neuropsychiatric Disorder Associated with Streptoccocal Infections
(PANDAS).

When reference is made herein to using a multispecific or bispecific construct therapeutically to treat an autoimmune disorder and disease shown in Table 1, this means that the multispecific or bispecific construct can be used to treat SLE, and/or SLE flare-ups, and/or lupus nephritis, and/or acute GvHD, and/or chronic GvHD, and/or RA, and/or Pemphigus, and/or PVu, and/or PVe, and/or PE, and/or PF, and/or CLL, and/or GPA, and/or MPA, and/or MS, and/or PPMS, and/or RMS, and/or ANCA-associated vasculitis, and/or NHL, and/or Rassmussen's encephalitis, and/or ITP, and/or FLL, and/or immune conditions treatable by BCDT, and/or IBD, and/or UC, and/or CD, and/or PANS, and/or PANDAS.

### Experimental

Having generally described embodiments drawn to constructs for binding to CD68-expressing, ARID3a-producing naïve B cells, nucleic acids which encode such constructs, vectors and host cells comprising said nucleic acids which encode such constructs, and methods of treating certain disorders with such constructs, a fuller understanding can be obtained by reference to certain specific examples which are provided below for purposes of illustration only and are not intended to be limiting.

### Methods

CD68 is a surface marker that identifies ARID3a-expressing SLE naïve B cells

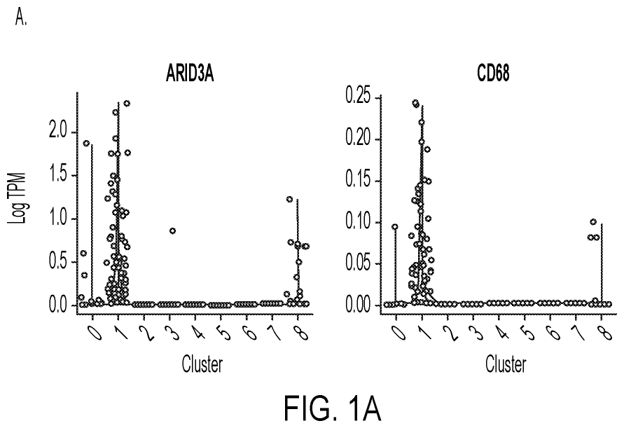

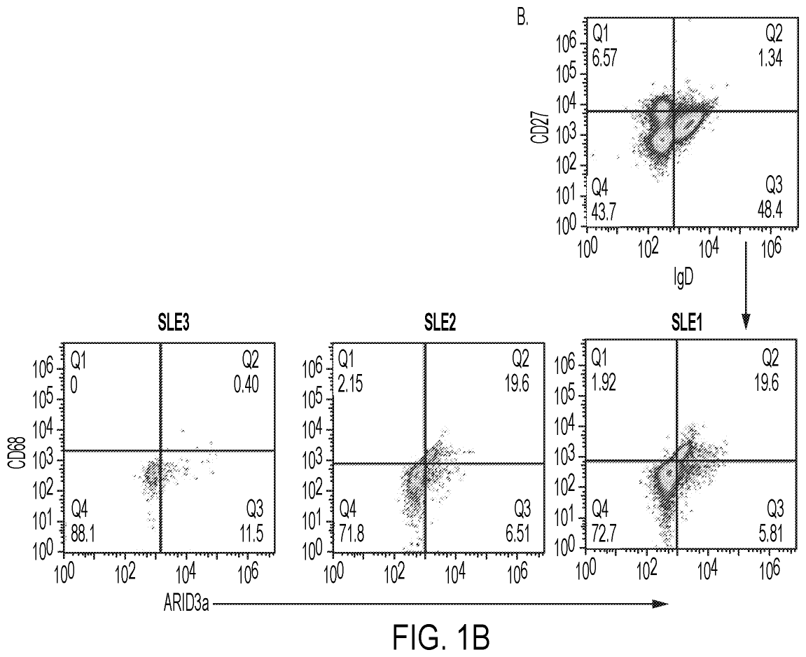

CD68 transcripts were identified by scRNA-seq cells within the three UMAP clusters that express ARID3a (FIG. 1(A)). Flow cytometry of naïve B cells from three randomly chosen patients (SLE1-3) indicate ARID3a⁺ cells co-express surface CD68, while ARID3a naïve cells do not (FIG. 1(B)). Numbers of ARID3a⁺ naïve B cells are highly variable (FIG. 1(B), application). While CD68 is generally thought of as a monocyte marker/scavenger receptor, others found select anti-CD68 monoclonal Abs stained some human lymphocytes (Gottfried, E., et al., 2008. Scand J Immunol 67: 453-463), and a subset of mouse marginal zone B cells is CD68⁺ (Zhang, P., et al., 2007. Mol. Immunol. 44:332-337). These data validate transcription data at the protein level for CD68.

Importantly, CD68 surface expression can be used to sort ARID3a⁺ and ARID3a naïve B cells from the same patients, allowing use of samples with 50% ARID3a⁺ naïve B cells. This will significantly reduce effects from patient-specific ethnic/genetic variabilities introduced by comparing patients with high numbers of ARID3a⁺ cells to those with >10% ARID3a⁺ naïve B cells.

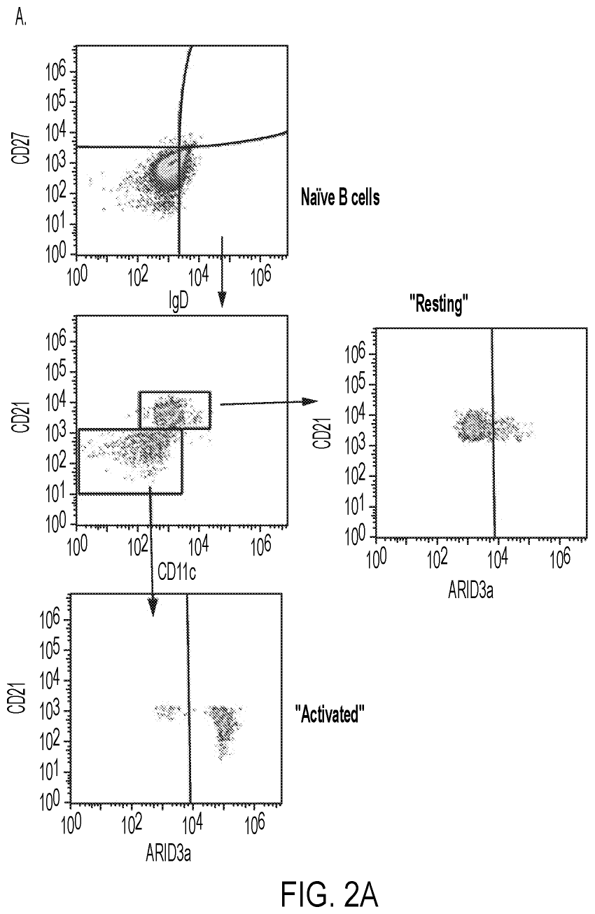

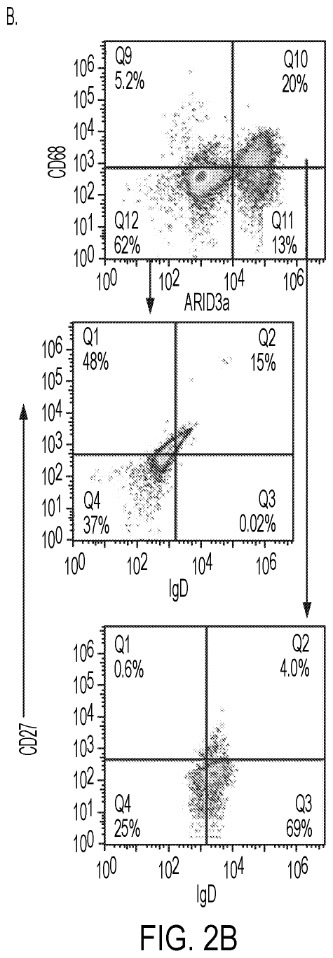

Experiments from the Sanz lab (Jenks et al. 2018, op.cit.) were reproduced, generating activated naïve B cells from healthy controls, the precursors of mature pathogenic DN B cells that are expanded in SLE patients. Those in vitro activated naïve B cells express ARID3a (FIG. 2(A)). The “resting” naïve B cells that maintained CD21 (CR2/EBV receptor) expression were ARID3a negative (ARID3a⁻). These data support the assertion that ARID3a expression in naïve B cells contributes to SLE pathogenesis. CD68 is also co-expressed with ARID3a in 3 day-stimulated “activated” naïve B cells (FIG. 2(B)), but is lost in more mature IgD-DN-like cells that maintain ARID3a expression and begin to be apparent at day 3 (lower right quadrant of top panel, FIG. 2(B)). Seven-day cultures induced CD11c expression and DN2 cells (ref. 15) with ARID3a expression (N=1, not shown). ARID3a expression was thus induced in naïve B cells in vitro.

Naïve B cells from SLE patients express ARID3a and the surface marker CD68. These cells mature into ARID3a-expressing mature B cells that are associated with increases in SLE disease activity, but that now lack CD68. By eliminating the B cells at the naïve, pre-mature, stage autoimmune responses to SLE will be reduced.

ARID3a has been shown to be necessary for responses to Streptococcus pneumoniae in mice. The syndrome PANDAS (Pediatric autoimmune neuropsychiatric disorders associated with streptococcal infections) is an autoimmune disease hypothesized to show increased numbers of ARID3a-expressing, CD68⁺ naïve B cells. Data from two distinct PANDAS patients PANDAS patients showed increased numbers of ARID3a- and CD68-expressing naïve B cells. By using a flow cytometric assay, children can be screened for developing autoimmune cells, and the bispecific constructs of the present disclosure can be used to eliminate those cells and reduce or eliminate the effects of PANDAS.

In particular but non-limiting embodiments, a bispecific antibody comprising an anti-CD20 arm derived from Rituximab (as described above) and an anti-CD68 arm derived from the heavy and light chain sequences and CDRs from the Y182A anti-CD68 protein or the KP-1 anti-CD68 protein (as described above) can be produced. This bispecific antibody may have a mouse IgG2a constant region heavy chain, or may be a humanized antibody construct comprising the same anti-CD20 and anti-CD68 arms.

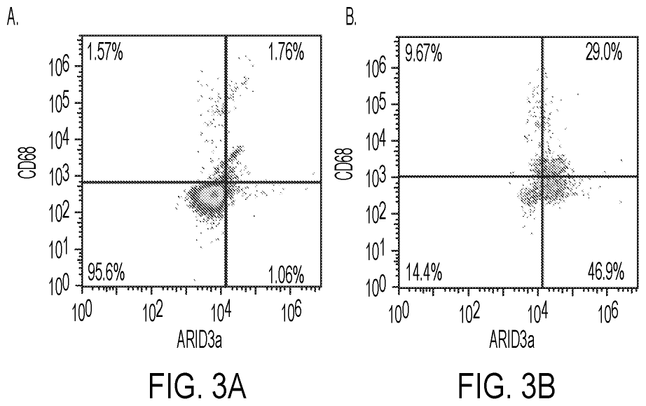

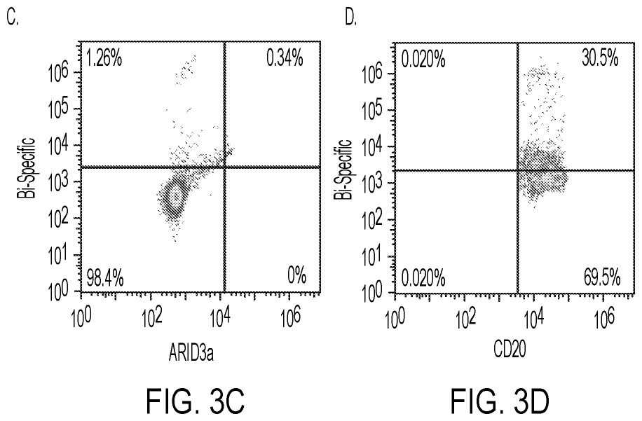

The results shown in FIG. 3 demonstrate that the bispecific construct of the present disclosure recognizes and binds to a subset of ARID3a⁺ naïve B cells (FIG. 3D) that is a similar percentage as that recognized by the native Y182A anti-CD68 antibody in FIG. 3B. Furthermore, there was no significant reactivity of the bispecific construct with T cells (FIG. 3A, C). In addition, the bispecific construct did not bind to all CD20⁺ B cells as shown in FIG. 3D, indicating that: (1) the bispecific construct binds to the CD68⁺ B cells, and (2) that there is specificity as it did not react with all CD20⁺ B cells, but a subset thereof. These results shows that a bispecific construct comprising a binding arm which targets a B-cell-specific cell surface marker and a binding arm which targets CD68 protein will bind to the subset of B-cells which comprises ARID3a-expressing naïve B cells.

### Anti-CD20 Binding Arms

In at least some embodiments, the present disclosure is directed to a multispecific construct (e.g., a bispecific construct) comprising a CD20 binding arm comprising the CDRs of at least one of the anti-CD20 monoclonal antibodies selected from Rituximab, Obinutuzumab, Ocrelizumab, Ocaratuzumab, Ofatumumab, Tositumomab, Ublituximab, Ibritumomab, Veltuzumab, PRO131921, 7D8, 2F2, 11B8, and 2C6.

In at least some embodiments, a CD20 binding arm of the multispecific construct comprises the CDRs of the anti-CD20 monoclonal antibody rituximab. In at least some embodiments, a CD20 binding arm of the multispecific construct comprises the CDRs of the anti-CD20 monoclonal antibody obinutuzumab. In at least some embodiments, a CD20 binding arm of the multispecific construct comprises the CDRs of the anti-CD20 monoclonal antibody ofatumumab. In at least some embodiments, a CD20 binding arm of the multispecific construct comprises the CDRs of the anti-CD20 monoclonal antibody ocrelizumab. In at least some embodiments, a CD20 binding arm of the multispecific construct comprises the CDRs of the anti-CD20 monoclonal antibody veltuzumab. In at least some embodiments, a CD20 binding arm of the multispecific construct comprises the CDRs of the anti-CD20 monoclonal antibody ocaratuzumab. In at least some embodiments, a CD20 binding arm of the multispecific construct comprises the CDRs of the anti-CD20 monoclonal antibody PRO131921. In at least some embodiments, a CD20 binding arm of the multispecific construct comprises the CDRs of the anti-CD20 monoclonal antibody tositumomab. In at least some embodiments, a CD20 binding arm of the multispecific construct comprises the CDRs of the anti-CD20 monoclonal antibody ublituximab. In at least some embodiments, a CD20 binding arm of the multispecific construct comprises the CDRs of the anti-CD20 monoclonal antibody ibritumomab. In at least some embodiments, a CD20 binding arm of the multispecific construct comprises the CDRs of the anti-CD20 monoclonal antibody 7D8. In at least some embodiments, a CD20 binding arm of the multispecific construct comprises the CDRs of the anti-CD20 monoclonal antibody 2F2. In at least some embodiments, a CD20 binding arm of the multispecific construct comprises the CDRs of the anti-CD20 monoclonal antibody 11B8. In at least some embodiments, a CD20 binding arm of the multispecific construct comprises the CDRs of the anti-CD20 monoclonal antibody 2C6. The multispecific constructs described above can be used therapeutically as treatments for autoimmune disorders and diseases, including but not limited to, those shown in Table 1.

In a particular embodiment, for example, a CD20 binding arm of the multispecific construct comprises the CDRs of the anti-CD20 monoclonal antibody Rituximab, wherein the binding domain of the first binding arm comprises a CDRVL1 comprising the amino acid sequence RASSSVSYIHW (SEQ ID NO:1) or an amino acid sequence having at least 80% or at least 90% identity thereto (e.g., having 1 or 2 conservative amino acid substitutions); a CDRVL2 comprising the amino acid sequence ATSNLAS (SEQ ID NO:2), or an amino acid sequence having at least 80% or at least 90% identity thereto (e.g., having 1 or 2 conservative amino acid substitutions); a CDRVL3 comprising the amino acid sequence QQWTSNPPT (SEQ ID NO:3), or an amino acid sequence having at least 80% or at least 90% identity thereto (e.g., having 1 or 2 conservative amino acid substitutions); a CDRVH1 comprising the amino acid sequence SYNMH (SEQ ID NO:4), or an amino acid sequence having at least 80% or at least 90% identity thereto (e.g., having 1 or 2 conservative amino acid substitutions); a CDRVH2 comprising the amino acid sequence AIYPGNGDTSYNQKFKG (SEQ ID NO:5), or an amino acid sequence having at least 80% or at least 90% identity thereto (e.g., having 1 or 2 conservative amino acid substitutions); and a CDRVH3 comprising the amino acid sequence STYYGGDWYFNV (SEQ ID NO:6), or an amino acid sequence having at least 80% or at least 90% identity thereto (e.g., having 1 or 2 conservative amino acid substitutions). The multispecific constructs described above can be used therapeutically as treatments for autoimmune disorders and diseases, including but not limited to, those shown in Table 1.

In a particular embodiment, for example, a CD20 binding arm of the multispecific construct comprises the CDRs of the anti-CD20 monoclonal antibody Ocrelizumab, wherein the binding domain of the first binding arm comprises a CDRVL1 comprising the amino acid sequence RASSSVSYM (SEQ ID NO:7), or an amino acid sequence having at least 80% or at least 90% identity thereto (e.g., having 1 or 2 conservative amino acid substitutions); a CDRVL2 comprising the amino acid sequence APSNLAS (SEQ ID NO:8), or an amino acid sequence having at least 80% or at least 90% identity thereto; a CDRVL3 comprising the amino acid sequence QQWSFNPPT (SEQ ID NO:9), or an amino acid sequence having at least 80% or at least 90% identity thereto (e.g., having 1 or 2 conservative amino acid substitutions); a CDRVH1 comprising the amino acid sequence SYNMH (SEQ ID NO:4) or GYTFTSYNMH (SEQ ID NO:10), or an amino acid sequence having at least 80% or at least 90% identity thereto (e.g., having 1 or 2 conservative amino acid substitutions); a CDRVH2 comprising the amino acid sequence AIYPGNGDTSYNQKFKG (SEQ ID NO:11), or an amino acid sequence having at least 80% or at least 90% identity thereto (e.g., having 1 or 2 conservative amino acid substitutions); and a CDRVH3 comprising the amino acid sequence VVYYSNSYWYFDV (SEQ ID NO:12), or an amino acid sequence having at least 80% or at least 90% identity thereto (e.g., having 1 or 2 conservative amino acid substitutions). The multispecific constructs described above can be used therapeutically as treatments for autoimmune disorders and diseases, including but not limited to, those shown in Table 1.

Non-limiting examples of heavy and light variable regions that can be used in the anti-CD20 binding arms of the present constructs, or from which CDRs can be obtained for use in the constructs, are shown below.

In one embodiment, a CD20 binding arm of the multispecific construct of the present disclosure comprises the VH sequence and the VL sequence of the mAb Rituximab, wherein VH=QVQLQQPGAELVKPGASVKMSCKASGYTFTSYNMHWVKQTPGRGLEWIGAIY PGNGDTSYNQKFKGKATLTADKSSSTAYMQLSSLTSEDSAVYYCARSTYYGGDWY FNVWGAGTTVTVSA (SEQ ID NO:13), and VL=QIVLSQSPAILSASPGEKVTMTCRASSSVSYIHWFQQKPGSSPKPWIYATSNLASG VPVRFSGSGSGTSYSLTISRVEAEDAATYYCQQWTSNPPTFGGGTKLEIK (SEQ ID NO:14). These multispecific constructs can be used therapeutically as treatments for autoimmune disorders and diseases, including but not limited to, those shown in Table 1.

In one embodiment, a CD20 binding arm of the multispecific construct of the present disclosure comprises the VH sequence and the VL sequence of the mAb Obinutuzumab, wherein: VH=QVQLVQSGAEVKKPGSSVKVSCKASGYAFSYSWINWVRQAPGQGLEWMGRIF PGDGDTDYNGKFKGRVTITADKSTSTAYMELSSLRSEDTAVYYCARNVFDGYWLV YWGQGTLVTVSS (SEQ ID NO:15), and VL=DIVMTQTPLSLPVTPGEPASISCRSSKSLLHSNGITYLYWYLQKPGQSPQLLIYQM SNLVSGVPDRFSGSGSGTDFTLKISRVEAEDVGVYYCAQNLELPYTFGGGTKVEIK (SEQ ID NO:16). These multispecific constructs can be used therapeutically as treatments for autoimmune disorders and diseases, including but not limited to, those shown in Table 1.

In one embodiment, a CD20 binding arm of the multispecific construct of the present disclosure comprises the three CDRs of the Obinutuzumab VH, and the three CDRs of the Obinutuzumab VL. These multispecific constructs can be used therapeutically as treatments for autoimmune disorders and diseases, including but not limited to, those shown in Table 1.

In one embodiment, a CD20 binding arm of the multispecific construct of the present disclosure comprises the VH sequence and the VL sequence of the mAb Ocrelizumab, wherein: VH=EVQLVESGGGLVQPGGSLRLSCAASGYTFTSYNMHWVRQAPGKGLEWVGAIY PGNGDTSYNQKFKGRFTISVDKSKNTLYLQMNSLRAEDTAVYYCARVVYYSNSYW YFDVWGQGTLVTVSS (SEQ ID NO:17), and VL=DIQMTQSPSSLSASVGDRVTITCRASSSVSYMHWYQQKPGKAPKPLIYAPSNLAS GVPSRFSGSGSGTDFTLTISSLQPEDFATYYCQQWSFNPPTFGQGTKVEIK (SEQ ID NO:18). These multispecific constructs can be used therapeutically as treatments for autoimmune disorders and diseases, including but not limited to, those shown in Table 1.

In one embodiment, a CD20 binding arm of the multispecific construct of the present disclosure comprises the three CDRs of the Ocrelizumab VH, and the three CDRs of the Ocrelizumab VL. These multispecific constructs can be used therapeutically as treatments for autoimmune disorders and diseases, including but not limited to, those shown in Table 1.

In one embodiment, a CD20 binding arm of the multispecific construct of the present disclosure comprises the VH sequence and the VL sequence of the mAb Ofatumumab, wherein VH=QLVESGGGLVQPGRSLRLSCAASGFTFNDYAMHWVRQAPGKGLEWVSTISWN SGSIGYADSVKGRFTISRDNAKKSLYLQMNSLRAEDTALYYCAKDIQYGNYYYGMD VWGQGTTVTVSS (SEQ ID NO:19), and VL=EIVLTQSPATLSLSPGERATLSCRASQSVSSYLAWYQQKPGQAPRLLIYDASNRA TGIPARFSGSGSGTDFTLTISSLEPEDFAVYYCQQRSNWPITFGQGTRLEIK (SEQ ID NO:20). These multispecific constructs can be used therapeutically as treatments for autoimmune disorders and diseases, including but not limited to, those shown in Table 1.

In one embodiment, a CD20 binding arm of the multispecific construct of the present disclosure comprises the three CDRs of the Ofatumumab VH, and the three CDRs of the Ofatumumab VL. These multispecific constructs can be used therapeutically as treatments for autoimmune disorders and diseases, including but not limited to, those shown in Table 1.

In one embodiment, a CD20 binding arm of the multispecific construct of the present disclosure comprises the three CDRs of the Rituximab VH, and the three CDRs of the Rituximab VL. In one embodiment CDRVL1 is RASSSVSYIHW (SEQ ID NO:1), CDRVL2 is ATSNLAS (SEQ ID NO:2), CDRVL3 is QQWTSNPPT (SEQ ID NO:3), CDRVH1 is SYNMH (SEQ ID NO:4), CDRVH2 is AIYPGNGDTSYNQKFKG (SEQ ID NO:5), and CDRVH3 is STYYGGDWYFNV (SEQ ID NO:6). These multispecific constructs can be used therapeutically as treatments for autoimmune disorders and diseases, including but not limited to, those shown in Table 1.

In one embodiment, a CD20 binding arm of the multispecific construct of the present disclosure comprises the VH sequence and the VL sequence of the mAb Tositumomab, wherein VH=EVQLLESGGGLVQPGGSLRLSCAASGFTFSNYAMSWVRQAPGKGLEWVSSISG SGDYTYYTDSVKGRFTISRDNSKNTLYLQMNSLRAEDTAVYYCARSPWGYYLDSW GQGTLVTVSS (SEQ ID NO:21), and VL=DIQMTQSPPSLSASAGDRVTITCRASQGISSRLAWYQQKPEKAPKSLIYAASSLQS GVPSRFSGSGSGTDFTLTISSLQPEDFATYYCQQYNSYPYTFGQGTKLEIK (SEQ ID NO:22). These multispecific constructs can be used therapeutically as treatments for autoimmune disorders and diseases, including but not limited to, those shown in Table 1.

In one embodiment, a CD20 binding arm of the multispecific construct of the present disclosure comprises the three CDRs of the Tositumomab VH, and the three CDRs of the Tositumomab VL. These multispecific constructs can be used therapeutically as treatments for autoimmune disorders and diseases, including but not limited to, those shown in Table 1.

In one embodiment, a CD20 binding arm of the multispecific construct of the present disclosure comprises the VH sequence and the VL sequence of the mAb Ublituximab, wherein VH=QAYLQQSGAELVRPGASVKMSCKASGYTFTSYNMHWVKQTPRQGLEWIGGIY PGNGDTSYNQKFKGKATLTVGKSSSTAYMQLSSLTSEDSAVYFCARYDYNYAMDY WGQGTSVTVSS (SEQ ID NO:23), and VL=QIVLSQSPAILSASPGEKVTMTCRASSSVSYMHWYQQKPGSSPKPWIYATSNLAS GVPARFSGSGSGTSYSFTISRVEAEDAATYYCQQWTFNPPTFGGGTRLEIK (SEQ ID NO:24). These multispecific constructs can be used therapeutically as treatments for autoimmune disorders and diseases, including but not limited to, those shown in Table 1.

In one embodiment, a CD20 binding arm of the multispecific construct of the present disclosure comprises the three CDRs of the Ublituximab VH, and the three CDRs of the Ublituximab VL. These multispecific constructs can be used therapeutically as treatments for autoimmune disorders and diseases, including but not limited to, those shown in Table 1.

In one embodiment, a CD20 binding arm of the multispecific construct of the present disclosure comprises the VH sequence and the VL sequence of the mAb Veltuzumab, wherein VH=QVQLQQSGAEVKKPGSSVKVSCKASGYTFTSYNMHWVKQAPGQGLEWIGAIY PGMGDTSYNQKFKGKATLTADESTNTAYMELSSLRSEDTAFYYCARSTYYGGDWY FDVWGQGTTVTVSS (SEQ ID NO:25), and VL=DIQLTQSPSSLSASVGDRVTMTCRASSSVSYIHWFQQKPGKAPKPWIYATSNLAS GVPVRFSGSGSGTDYTFTISSLQPEDIATYYCQQWTSNPPTFGGGTKLEIK (SEQ ID NO:26). These multispecific constructs can be used therapeutically as treatments for autoimmune disorders and diseases, including but not limited to, those shown in Table 1.

In one embodiment, a CD20 binding arm of the multispecific construct of the present disclosure comprises the three CDRs of the Veltuzumab VH, and the three CDRs of the Veltuzumab VL. These multispecific constructs can be used therapeutically as treatments for autoimmune disorders and diseases, including but not limited to, those shown in Table 1.

In one embodiment, a CD20 binding arm of the multispecific construct of the present disclosure comprises a heavy variable chain from the anti-CD20 mAb 7D8, designated herein as 1H_{7D8}, which comprises the amino acid sequence: EVQLVESGGGLVQPDRSLRLSCAASGFTFHDYAMHWVRQAPGKGLEWVSTISWNS GTIGYADSVKGRFTISRDNAKNSLYLQMNSLRAEDTALYYCAKDIQYGNYYYGMD VWGQGTTVTVSS (SEQ ID NO:27). These multispecific constructs can be used therapeutically as treatments for autoimmune disorders and diseases, including but not limited to, those shown in Table 1.

In one embodiment the CD20 binding arm of the multispecific construct of the present disclosure comprises a light variable chain from anti-CD20 mAb 7D8, designated herein as 1L_{7D8}, which comprises the amino acid sequence: EIVLTQSPATLSLSPGERATLSCRASQSVSSYLAWYQQKPGQAPRLLIYDASNRATGI PARFSGSGSGTDFTLTISSLEPEDFAVYYCQQRSNWPITFGQGTRLEIK (SEQ ID NO:28). These multispecific constructs can be used therapeutically as treatments for autoimmune disorders and diseases, including but not limited to, those shown in Table 1.

In 1L_{7D8} VL CDR1=QSVSSY (SEQ ID NO:35), VL CDR2=DAS (SEQ ID NO:36), and VL CDR3=QQRSNWPIT (SEQ ID NO:37). In 1H_{7D8} VH CDR1=GFTFHDYA (SEQ ID NO:38), VH CDR2=ISWNSGTI (SEQ ID NO:39), and VH CDR3=AKDIQYGNYYYGMDV (SEQ ID NO:40).

In one embodiment the CD20 binding arm of the multispecific construct of the present disclosure comprises a heavy variable chain from anti-CD20 mAb 2F2, designated herein as 2H_{2F2}, which comprises the amino acid sequence: EVQLVESGGGLVQPGRSLRLSCAASGFTFNDYAMHWVRQAPGKGLEWVSTISWNS GSIGYADSVKGRFTISRDNAKKSLYLQMNSLRAEDTALYYCAKDIQYGNYYYGMD VWGQGTTVTVSS (SEQ ID NO:29). These multispecific constructs can be used therapeutically as treatments for autoimmune disorders and diseases, including but not limited to, those shown in Table 1.

In one embodiment the CD20 binding arm of the multispecific construct of the present disclosure comprises a light variable chain from anti-CD20 mAb 2F2, designated herein as 2L_{2F2}, which comprises the amino acid sequence: EIVLTQSPATLSLSPGERATLSCRASQSVSSYLAWYQQKPGQAPRLLIYDASNRATGI PARFSGSGSGTDFTLTISSLEPEDFAVYYCQQRSNWPITFGQGTRLEIK (SEQ ID NO:30). These multispecific constructs can be used therapeutically as treatments for autoimmune disorders and diseases, including but not limited to, those shown in Table 1.

In 2L_{2F2} VL CDR1=QSVSSY (SEQ ID NO:35), VL CDR2=DAS (SEQ ID NO:36), and VL CDR3=QQRSNWPIT (SEQ ID NO:37). In 2H2F2 VH CDR1=GFTFNDYA (SEQ ID NO:41), VH CDR2=ISWNSGSI (SEQ ID NO:42), and VH CDR3=AKDIQYGNYYYGMDV (SEQ ID NO:40).

In one embodiment the CD20 binding arm of the multispecific construct of the present disclosure comprises a heavy variable chain from anti-CD20 mAb 11B8, designated herein as 3H_{11B8}, which comprises the amino acid sequence: EVQLVQSGGGLVHPGGSLRLSCTGSGFTFSYHAMHWVRQAPGKGLEWVSIIGTGGV TYYADSVKGRFTISRDNVKNSLYLQMNSLRAEDMAVYYCARDYYGAGSFYDGLYG MDVWGQGTTVTVSS (SEQ ID NO:31). These multispecific constructs can be used therapeutically as treatments for autoimmune disorders and diseases, including but not limited to, those shown in Table 1.

In one embodiment the CD20 binding arm of the multispecific construct of the present disclosure comprises a light variable chain from anti-CD20 mAb 11B8, designated herein as 3L_{11B8}, which comprises the amino acid sequence: EIVLTQSPATLSLSPGERATLSCRASQSVSSYLAWYQQKPGQAPRLLIYDASNRATGI PARFSGSGSGTDFTLTISSLEPEDFAVYYCQQRSDWPLTFGGGTKVEIK (SEQ ID NO:32). These multispecific constructs can be used therapeutically as treatments for autoimmune disorders and diseases, including but not limited to, those shown in Table 1.

In 3L_{11B8} VL CDR1=QSVSSY (SEQ ID NO:35), VL CDR2=DAS (SEQ ID NO:36), and VL CDR3=QQRSDWPLT (SEQ ID NO:43). In 3H_{11B8} VH CDR1=GFTFSYHA (SEQ ID NO:44), VH CDR2=IGTGGVT (SEQ ID NO:45), and VH CDR3=ARDYYGAGSFYDGLYGMDV (SEQ ID NO:46).

In one embodiment the CD20 binding arm of the multispecific construct of the present disclosure comprises a heavy variable chain from anti-CD20 mAb 2C6, designated herein as 4H_{2C6}, which comprises the amino acid sequence: AVQLVESGGGLVQPGRSLRLSCAASGFTFGDYTMHWVRQAPGKGLEWVSGISWNS GSIGYADSVKGRFTISRDNAKNSLYLQMNSLRAEDTALYYCTKDNQYGSGSTYGLG VWGQGTLVT VSS (SEQ ID NO:33). These multispecific constructs can be used therapeutically as treatments for autoimmune disorders and diseases, including but not limited to, those shown in Table 1.

In one embodiment the CD20 binding arm of the multispecific construct of the present disclosure comprises a light variable chain from anti-CD20 mAb 2C6, designated herein as 4L_{2C6}, which comprises the amino acid sequence: EIVLTQSPATLSLSPGERATLSCRASQSVSSYLAWYQQKPGQAPRLLIYDASNRATGI PARFSGSGSGTDFTLTISSLEPEDFAVYYCQQRSNWPLTFGGGTKVEIKIHI (SEQ ID NO:34). These multispecific constructs can be used therapeutically as treatments for autoimmune disorders and diseases, including but not limited to, those shown in Table 1.

In 4L_{2C6} VL CDR1=QSVSSY (SEQ ID NO:35), VL CDR2=DAS (SEQ ID NO:36), and VL CDR3=QQRSNWPLT (SEQ ID NO:47). In 4H2c6 VH CDR1=GFTFGDYT (SEQ ID NO:48), VH CDR2=ISWNSGSI (SEQ ID NO:42), and VH CDR3=TKDNQYGSGSTYGLGV (SEQ ID NO:49).

In the below, the following notation is used: 1_{H7D8}=1H, 2H_{2F2}=2H, 3H_{11B8}=3H, 4H_{2C6}=4H, 1L_{7D8}=1L, 2L_{2F2}=2L, 3L_{11B8}=3L, 4L_{2C6}=4L.

In one embodiment the CD20 binding arm of the multispecific construct of the present disclosure, hereby designated as 1H1L, comprises the heavy variable chain 1H and the light variable chain 1L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 1H2L, comprises the heavy variable chain 1H and the light variable chain 2L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 1H3L, comprises the heavy variable chain 1H and the light variable chain 3L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 1H4L, comprises the heavy variable chain 1H and the light variable chain 4L. These multispecific constructs can be used therapeutically as treatments

In one embodiment the CD20 binding arm of the multispecific construct of the present disclosure, hereby designated as 2H1L, comprises the heavy variable chain 2H and the light variable chain 1L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 3H1L, comprises the heavy variable chain H and the light variable chain 1L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 4H1L, comprises the heavy variable chain 4H and the light variable chain 1L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 2H2L, comprises the heavy variable chain 2H and the light variable chain 2L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 2H3L, comprises the heavy variable chain 2H and the light variable chain 3L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 2H4L, comprises the heavy variable chain 2H and the light variable chain 4L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 3H2L, comprises the heavy variable chain 3H and the light variable chain 2L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 4H2L, comprises the heavy variable chain 4H and the light variable chain 2L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 3H3L, comprises the heavy variable chain 3H and the light variable chain 3L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 3H4L, comprises the heavy variable chain 3H and the light variable chain 4L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 4H3L, comprises the heavy variable chain 4H and the light variable chain 3L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 4H4L, comprises the heavy variable chain 4H and the light variable chain 4L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 1H1L_{CDRs}, comprises the 3 CDRs of the heavy variable chain 1H and the 3 CDRs of the light variable chain 1L. In this embodiment, the multispecific construct comprises a CD20 binding arm comprising VH CDR1-3 and VL CDR1-3 wherein VH CDR1=GFTFNDYA (SEQ ID NO:41), VH CDR2=ISWNSGSI (SEQ ID NO:42), VH CDR3=AKDIQYGNYYYGMDV (SEQ ID NO:40), VL CDR1=QSVSSY (SEQ ID NO:35), VL CDR2=DAS (SEQ ID NO:36), and VL CDR3=QQRSNWPIT (SEQ ID NO:37). These multispecific constructs can be used therapeutically as treatments for autoimmune disorders and diseases, including but not limited to, those shown in Table 1.

In one embodiment the CD20 binding arm of the multispecific construct of the present disclosure, hereby designated as 1H2L_{CDRs}, comprises the 3 CDRs of heavy variable chain 1H and the 3 CDRs of light variable chain 2L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 1H3L_{CDRs}, comprises the 3 CDRs of heavy variable chain 1H and the 3 CDRs of light variable chain 3L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 1H4L_{CDRs}, comprises the 3 CDRs of the heavy variable chain 1H and the 3 CDRs of the light variable chain 4L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 2H1L_{CDRs}, comprises the 3 CDRs of the heavy variable chain 2H and the 3 CDRs of the light variable chain 1L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 3H1L_{CDRs}, comprises the 3 CDRs of the heavy variable chain H and the 3 CDRs of the light variable chain 1L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 4H1L_{CDRs}, comprises the 3 CDRs of the heavy variable chain 4H and the 3 CDRs of the light variable chain 1L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 2H2L_{CDRs}, comprises the 3 CDRs of the heavy variable chain 2H and the 3 CDRs of the light variable chain 2L. In this embodiment, the multispecific construct comprises a CD20 binding arm comprising VH CDR1-3 and VL CDR1-3 wherein VH CDR1=GFTFNDYA (SEQ ID NO:41), VH CDR2=ISWNSGSI (SEQ ID NO:42), VH CDR3=AKDIQYGNYYYGMDV (SEQ ID NO:40), VL CDR1=QSVSSY (SEQ ID NO:35), VL CDR2=DAS (SEQ ID NO:36), and VL CDR3=QQRSNWPIT (SEQ ID NO:37). These multispecific constructs can be used therapeutically as treatments for autoimmune disorders and diseases, including but not limited to, those shown in Table 1.

In one embodiment the CD20 binding arm of the multispecific construct of the present disclosure, hereby designated as 2H3L_{CDRs}, comprises the 3 CDRs of the heavy variable chain 2H and the 3 CDRs of the light variable chain 3L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 2H4L_{CDRs}, comprises the 3 CDRs of the heavy variable chain 2H and the 3 CDRs of the light variable chain 4L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 3H2L_{CDRs}, comprises the 3 CDRs of the heavy variable chain 3H and the 3 CDRs of the light variable chain 2L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 4H2L_{CDRs}, comprises the 3 CDRs of the heavy variable chain 4H and the 3 CDRs of the light variable chain 2L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 3H3L_{CDRs}, comprises the 3 CDRs of the heavy variable chain 3H and the 3 CDRs of the light variable chain 3L. In this embodiment, the multispecific construct comprises a CD20 binding arm comprising VH CDR1-3 and VL CDR1-3 wherein VH CDR1=GFTFSYHA (SEQ ID NO:44), VH CDR2=IGTGGVT (SEQ ID NO:45), VH CDR3=ARDYYGAGSFYDGLYGMDV (SEQ ID NO:46), VL CDR1=QSVSSY (SEQ ID NO:35), VL CDR2=DAS (SEQ ID NO:36), and VL CDR3=QQRSDWPLT (SEQ ID NO:43). These multispecific constructs can be used therapeutically as treatments for autoimmune disorders and diseases, including but not limited to, those shown in Table 1.

In one embodiment the CD20 binding arm of the multispecific construct of the present disclosure, hereby designated as 3H4L_{CDRs}, comprises the 3 CDRs of the heavy variable chain 3H and the 3 CDRs of the light variable chain 4L. These multispecific constructs can be used therapeutically as treatments for autoimmune disorders and diseases, including but not limited to, those shown in Table 1.

In one embodiment the CD20 binding arm of the multispecific construct of the present disclosure, hereby designated as 4H3L_{CDRs}, comprises the 3 CDRs of the heavy variable chain 4H and the 3 CDRs of the light variable chain 3L. These multispecific constructs can be used therapeutically as treatments for autoimmune disorders and diseases, including but not limited to, those shown in Table 1.

In one embodiment the CD2 binding arm of the multispecific construct of the present disclosure, hereby designated as 4H4L_{CDRs}, comprises the 3 CDRs of the heavy variable chain 4H and the 3 CDRs of the light variable chain 4L. In this embodiment, the multispecific construct comprises a CD2 binding arm comprising VH CDR1-3 and VL CDR1-3 wherein VH CDR1=GFTFGDYT (SEQ ID NO:48), VH CDR2=ISWNSGSI (SEQ ID NO:42), VH CDR3=TKDNQYGSGSTYGLGV (SEQ ID NO:49), VL CDR1=QSVSSY (SEQ ID NO:35), VL CDR2=DAS (SEQ ID NO:36), and VL CDR3=QQRSNWPLT (SEQ ID NO:47). These multispecific constructs can be used therapeutically as treatments for autoimmune disorders and diseases, including but not limited to, those shown in Table 1.

Other anti-CD20 monoclonal antibodies whose variable heavy and light regions, and VH and VL CDRs thereof, can be used to form multispecific constructs for use in the presently disclosed methods include but are not limited to Ocaratuzumab, PRV131921, and Ibritumomab. These multispecific constructs can be used therapeutically as treatments for autoimmune disorders and diseases, including but not limited to, those shown in Table 1.

Table 2 shows CDR sequences of various anti-CD20 mAbs described above.

TABLE 2
CDR sequences of various anti-CD20 mAbs

| Amino acid sequence | SEQ ID NO: | Source antibody | CDR Descriptor |
|---|---|---|---|
| RASSSVSYIHW | SEQ ID NO: 1 | Rituxumab | CDRVL1 |
| ATSNLAS | SEQ ID NO: 2 | Rituxumab | CDRVL2 |
| QQWTSNPPT | SEQ ID NO: 3 | Rituxumab | CDRVL3 |
| SYNMH | SEQ ID NO: 4 | Rituxumab | CDRVH1 |
| AIYPGNGDTSYNQKFKG | SEQ ID NO: 5 | Rituxumab | CDRVH2 |
| STYYGGDWYFNV | SEQ ID NO: 6 | Rituxumab | CDRVH3 |
| RASSSVSYM | SEQ ID NO: 7 | Ocrelizumab | CDRVL1 |
| APSNLAS | SEQ ID NO: 8 | Ocrelizumab | CDRVL2 |
| QQWSFNPPT | SEQ ID NO: 9 | Ocrelizumab | CDRVL3 |
| GYTFTSYNMH | SEQ ID NO: 10 | Ocrelizumab | CDRVH1 |
| AIYPGNGDTSYNQKFKG | SEQ ID NO: 11 | Ocrelizumab | CDRVH2 |
| VVYYSNSYWYFDV | SEQ ID NO: 12 | Ocrelizumab | CDRVH3 |
| QSVSSY | SEQ ID NO: 35 | 7D8; 2F2; 11B8; 2C6 | CDRVL1 |
| DAS | SEQ ID NO: 36 | 7D8; 2F2; 11B8; 2C6 | CDRVL2 |
| QQRSNWPIT | SEQ ID NO: 37 | 7D8; 2F2 | CDRVL3 |
| GFTFHDYA | SEQ ID NO: 38 | 7D8 | CDRVH1 |
| ISWNSGTI | SEQ ID NO: 39 | 7D8 | CDRVH2 |
| AKDIQYGNYYYGMDV | SEQ ID NO: 40 | 7D8; 2F2 | CDRVH3 |
| GFTFNDYA | SEQ ID NO: 41 | 2F2 | CDRVH1 |
| ISWNSGSI | SEQ ID NO: 42 | 2F2; 2C6 | CDRVH2 |
| QQRSDWPLT | SEQ ID NO: 43 | 11B8; | CDRVL2 |
| GFTFSYHA | SEQ ID NO: 44 | 11B8 | CDRVH1 |
| IGTGGVT | SEQ ID NO: 45 | 11B8 | CDRVH2 |
| ARDYYGAGSFYDGLYGMDV | SEQ ID NO: 46 | 11B8 | CDRVH3 |
| QQRSNWPLT | SEQ ID NO: 47 | 2C6 | CDRVL3 |
| GFTFGDYT | SEQ ID NO: 48 | 2C6 | CDRVH1 |
| TKDNQYGSGSTYGLGV | SEQ ID NO: 49 | 2C6 | CDRVH3 |

Table 3 shows Variable light (VL) and Variable heavy (VH) sequences of certain anti-CD20 mAbs described above.

TABLE 3
Variable light (VL) and Variable heavy (VH) sequences of certain anti-CD20 mAbs

| | | | Sequence |
|---|---|---|---|
| Amino acid sequence | SEQ ID NO: | Source Mab | Descriptor |
| QVQLQQPGAELVKPGASVKMSCKASGYTFTS | SEQ ID NO: 13 | Rituxumab | VH chain |
| YNMHWVKQTPGRGLEWIGAIYPGNGDTSYN | | | |
| QKFKGKATLTADKSSSTAYMQLSSLTSEDSA | | | |
| VYYCARSTYYGGDWYFNVWGAGTTVTVSA | | | |
| QIVLSQSPAILSASPGEKVTMTCRASSSVSYIH | SEQ ID NO: 14 | Rituxumab | VL chain |
| WFQQKPGSSPKPWIYATSNLASGVPVRFSGSG | | | |
| SGTSYSLTISRVEAEDAATYYCQQWTSNPPTF | | | |
| GGGTKLEIK | | | |
| QVQLVQSGAEVKKPGSSVKVSCKASGYAFSY | SEQ ID NO: 15 | Obinutuzumab | VH chain |
| SWINWVRQAPGQGLEWMGRIFPGDGDTDYN | | | |
| GKFKGRVTITADKSTSTAYMELSSLRSEDTAV | | | |
| YYCARNVFDGYWLVYWGQGTLVTVSS | | | |
| DIVMTQTPLSLPVTPGEPASISCRSSKSLLHSN | SEQ ID NO: 16 | Obinutuzumab | VL chain |
| GITYLYWYLQKPGQSPQLLIYQMSNLVSGVP | | | |
| DRFSGSGSGTDFTLKISRVEAEDVGVYYCAQ | | | |
| NLELPYTFGGGTKVEIK | | | |
| EVQLVESGGGLVQPGGSLRLSCAASGYTFTS | SEQ ID NO: 17 | Ocrelizumab | VH chain |
| YNMHWVRQAPGKGLEWVGAIYPGNGDTSY | | | |
| NQKFKGRFTISVDKSKNTLYLQMNSLRAEDT | | | |
| AVYYCARVVYYSNSYWYFDVWGQGTLVTV | | | |
| SS | | | |
| DIQMTQSPSSLSASVGDRVTITCRASSSVSYM | SEQ ID NO: 18 | Ocrelizumab | VL chain |
| HWYQQKPGKAPKPLIYAPSNLASGVPSRFSGS | | | |
| GSGTDFTLTISSLQPEDFATYYCQQWSFNPPTF | | | |
| GQGTKVEIK | | | |
| QLVESGGGLVQPGRSLRLSCAASGFTFNDYA | SEQ ID NO: 19 | Ofatumumab | VH chain |
| MHWVRQAPGKGLEWVSTISWNSGSIGYADS | | | |
| VKGRFTISRDNAKKSLYLQMNSLRAEDTALY | | | |
| YCAKDIQYGNYYYGMDVWGQGTTVTVSS | | | |
| EIVLTQSPATLSLSPGERATLSCRASQSVSSYL | SEQ ID NO: 20 | Ofatumumab | VL chain |
| AWYQQKPGQAPRLLIYDASNRATGIPARFSGS | | | |
| GSGTDFTLTISSLEPEDFAVYYCQQRSNWPITF | | | |
| GQGTRLEIK | | | |
| EVQLLESGGGLVQPGGSLRLSCAASGFTFSNY | SEQ ID NO: 21 | Tositumomab | VH chain |
| AMSWVRQAPGKGLEWVSSISGSGDYTYYTD | | | |
| SVKGRFTISRDNSKNTLYLQMNSLRAEDTAV | | | |
| YYCARSPWGYYLDSWGQGTLVTVSS | | | |
| DIQMTQSPPSLSASAGDRVTITCRASQGISSRL | SEQ ID NO: 22 | Tositumomab | VL chain |
| AWYQQKPEKAPKSLIYAASSLQSGVPSRESGS | | | |
| GSGTDFTLTISSLQPEDFATYYCQQYNSYPYT | | | |
| FGQGTKLEIK | | | |
| QAYLQQSGAELVRPGASVKMSCKASGYTFTS | SEQ ID NO: 23 | Ublituximab | VH chain |
| YNMHWVKQTPRQGLEWIGGIYPGNGDTSYN | | | |
| QKFKGKATLTVGKSSSTAYMQLSSLTSEDSA | | | |
| VYFCARYDYNYAMDYWGQGTSVTVSS | | | |
| QIVLSQSPAILSASPGEKVTMTCRASSSVSYM | SEQ ID NO: 24 | Ublituximab | VL chain |
| HWYQQKPGSSPKPWIYATSNLASGVPARFSG | | | |
| SGSGTSYSFTISRVEAEDAATYYCQQWTFNPP | | | |
| TFGGGTRLEIK | | | |
| QVQLQQSGAEVKKPGSSVKVSCKASGYTFTS | SEQ ID NO: 25 | Veltuzumab | VH chain |
| YNMHWVKQAPGQGLEWIGAIYPGMGDTSYN | | | |
| QKFKGKATLTADESTNTAYMELSSLRSEDTA | | | |
| FYYCARSTYYGGDWYFDVWGQGTTVTVSS | | | |
| DIQLTQSPSSLSASVGDRVTMTCRASSSVSYIH | SEQ ID NO: 26 | Veltuzumab | VL chain |
| WFQQKPGKAPKPWIYATSNLASGVPVRESGS | | | |
| GSGTDYTFTISSLQPEDIATYYCQQWTSNPPTF | | | |
| GGGTKLEIK | | | |
| EVQLVESGGGLVQPDRSLRLSCAASGFTFHD | SEQ ID NO: 27 | 7D8 | VH chain |
| YAMHWVRQAPGKGLEWVSTISWNSGTIGYA | | | |
| DSVKGRFTISRDNAKNSLYLQMNSLRAEDTA | | | |
| LYYCAKDIQYGNYYYGMDVWGQGTTVTVSS | | | |
| EIVLTQSPATLSLSPGERATLSCRASQSVSSYL | SEQ ID NO: 28 | 7D8 | VL chain |
| AWYQQKPGQAPRLLIYDASNRATGIPARFSGS | | | |
| GSGTDFTLTISSLEPEDFAVYYCQQRSNWPITF | | | |
| GQGTRLEIK | | | |
| EVQLVESGGGLVQPGRSLRLSCAASGFTEND | SEQ ID NO: 29 | 2F2 | VH chain |
| YAMHWVRQAPGKGLEWVSTISWNSGSIGYA | | | |
| DSVKGRFTISRDNAKKSLYLQMNSLRAEDTA | | | |
| LYYCAKDIQYGNYYYGMDVWGQGTTVTVSS | | | |
| EIVLTQSPATLSLSPGERATLSCRASQSVSSYL | SEQ ID NO: 30 | 2F2 | VL chain |
| AWYQQKPGQAPRLLIYDASNRATGIPARFSGS | | | |
| GSGTDFTLTISSLEPEDFAVYYCQQRSNWPITF | | | |
| GQGTRLEIK | | | |
| EVQLVQSGGGLVHPGGSLRLSCTGSGFTFSYH | SEQ ID NO: 31 | 11B8 | VH chain |
| AMHWVRQAPGKGLEWVSIIGTGGVTYYADS | | | |
| VKGRFTISRDNVKNSLYLQMNSLRAEDMAV | | | |
| YYCARDYYGAGSFYDGLYGMDVWGQGTTV | | | |
| TVSS | | | |
| EIVLTQSPATLSLSPGERATLSCRASQSVSSYL | SEQ ID NO: 32 | 11B8 | VL chain |
| AWYQQKPGQAPRLLIYDASNRATGIPARFSGS | | | |
| GSGTDFTLTISSLEPEDFAVYYCQQRSDWPLT | | | |
| FGGGTKVEIK | | | |
| AVQLVESGGGLVQPGRSLRLSCAASGFTFGD | SEQ ID NO: 33 | 2C6 | VH chain |
| YTMHWVRQAPGKGLEWVSGISWNSGSIGYA | | | |
| DSVKGRFTISRDNAKNSLYLQMNSLRAEDTA | | | |
| LYYCTKDNQYGSGSTYGLGVWGQGTLVT | | | |
| VSS | | | |
| EIVLTQSPATLSLSPGERATLSCRASQSVSSYL | SEQ ID NO: 34 | 3C6 | VL chain |
| AWYQQKPGQAPRLLIYDASNRATGIPARFSGS | | | |
| GSGTDFTLTISSLEPEDFAVYYCQQRSNWPLT | | | |
| FGGGTKVEIKIHI | | | |

Commercial sources of exemplary anti-CD20 mAbs are shown in Table 4 below.

TABLE 4
Exemplary Anti-CD20 monoclonal antibodies and sources

| Generic name or Identifier | Trade name |
|---|---|
| Obinutuzumab | Gazyva ® |
| Ocrelizumab | Ocrevus ® |
| Ocaratuzumab | |
| Ofatumumab | Kesimpta ® |
| Rituximab | Rituxan ® |
| Tositumomab | Bexxar ® |
| Ublituximab | |
| Ibritumomab | Zevalin ® |
| Veltuzumab | |
| PRO131921 | |
| 7D8 | |
| 2F2 | |
| 11B8 | |
| 2C6 | |

### Anti-CD68 Binding Arms

Examples of anti-CD68 monoclonal antibodies whose variable heavy and light regions, and VH and VL CDRs thereof, can be used to form multispecific constructs for use in the presently disclosed methods include but are not limited to Y1/82A; KP1; CD68/684; FA-11; EPR20545; EPR23917-164; EPR24100-133; EPR24100-8; SP-251; ED1; 3F7D3; PG-M1; CD68-2501; LAMP4-1830; 815CU17; 514H12; Ki-M7; 298807; Zr302; 3A9A7; OTI8A; OTI3b6; UMAB150;SN07-27; LAMP4-824; and C68-2908R. Commercial sources of the anti-CD68 mAbs are shown in Table 6 below. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of Y1/82A. These multispecific constructs can be used therapeutically as treatments for autoimmune disorders and diseases, including but not limited to, those shown Table 1.

In one embodiment the CD68 binding arm of the multispecific construct of the present disclosure comprises the VH and VL sequences, or the 3 VH CDRs and 3 VL CDRs, of CD68/684. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of KP1. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of FA-11. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of EPR20545. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of EPR23917-164. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of EPR24100-133. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of EPR24100-8. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of SP-251. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of ED1. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of 3F7D3. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of PG-M1. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of CD68-2501. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of LAMP4-1830. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of 815CU17. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of 514H12. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of Ki-M7. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of 298807. These multispecific constructs can be used therapeutically as treatments for autoimmune disorders and diseases, including but not limited to, those shown Table 1.

In one embodiment the CD68 binding arm of the multispecific construct of the present disclosure comprises the VH and VL sequences, or the 3 VH CDRs and 3 VL CDRs, of Zr302. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of 3A9A7. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of OTI8A. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of OTI3b6. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of UMAB150. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of SN07-27. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of LAMP4-824. These multispecific constructs can be used therapeutically as treatments for autoimmune disorders and diseases, including but not limited to, those shown in Table 1.

In one embodiment the CD68 binding arm of the multispecific construct of the present disclosure comprises the VH and VL sequences, or the 3 VH CDRs and 3 VL CDRs, of C68-2908R. These multispecific constructs can be used therapeutically as treatments for autoimmune disorders and diseases, including but not limited to, those shown in Table 1.

In a particular embodiment, for example, a CD68 binding arm of the multispecific construct comprises the CDRs of the anti-CD68 monoclonal antibody Y1/82A, wherein the binding domain of the first binding arm comprises a CDRVL1 comprising the amino acid sequence SASSSVSYMY (SEQ ID NO:50), or an amino acid sequence having at least 80% or at least 90% identity thereto (e.g., having 1 or 2 conservative amino acid substitutions); a CDRVL2 comprising the amino acid sequence RTSNLAS (SEQ ID NO:51), or an amino acid sequence having at least 80% or at least 90% identity thereto (e.g., having 1 or 2 conservative amino acid substitutions); a CDRVL3 comprising the amino acid sequence QQYHFFPPT (SEQ ID NO:52), or an amino acid sequence having at least 80% or at least 90% identity thereto (e.g., having 1 or 2 conservative amino acid substitutions); a CDRVH1 comprising the amino acid sequence GYTFINY (SEQ ID NO:53), or an amino acid sequence having at least 80% or at least 90% identity thereto (e.g., having 1 or 2 conservative amino acid substitutions); a CDRVH2 comprising the amino acid sequence LPGSHT (SEQ ID NO:54), or an amino acid sequence having at least 80% or at least 90% identity thereto (e.g., having 1 or 2 conservative amino acid substitutions); and a CDRVH3 comprising the amino acid sequence GGTRASFYYFDY (SEQ ID NO:55), or an amino acid sequence having at least 80% or at least 90% identity thereto (e.g., having 1 or 2 conservative amino acid substitutions). These multispecific constructs can be used therapeutically as treatments for autoimmune disorders and diseases, including but not limited to, those shown in Table 1.

In a particular embodiment, for example, a CD68 binding arm of the multispecific construct comprises the CDRs of the anti-CD68 monoclonal antibody C68/684, wherein the binding domain of the first binding arm comprises a CDRVL1 comprising the amino acid sequence RASKSVSTSGYSYMH (SEQ ID NO:56), or an amino acid sequence having at least 80% or at least 90% identity thereto (e.g., having 1 or 2 conservative amino acid substitutions); a CDRVL2 comprising the amino acid sequence LASNLES (SEQ ID NO:57), or an amino acid sequence having at least 80% or at least 90% identity thereto (e.g., having 1 or 2 conservative amino acid substitutions); a CDRVL3 comprising the amino acid sequence QHSRELPYT (SEQ ID NO:58), or an amino acid sequence having at least 80% or at least 90% identity thereto (e.g., having 1 or 2 conservative amino acid substitutions); a CDRVH1 comprising the amino acid sequence GYTFTNY (SEQ ID NO:59), or an amino acid sequence having at least 80% or at least 90% identity thereto (e.g., having 1 or 2 conservative amino acid substitutions); a CDRVH2 comprising the amino acid sequence YPGDGD (SEQ ID NO:60), or an amino acid sequence having at least 80% or at least 90% identity thereto (e.g., having 1 or 2 conservative amino acid substitutions); and a CDRVH3 comprising the amino acid sequence EPDDYDGYYYFDY (SEQ ID NO:61), or an amino acid sequence having at least 80% or at least 90% identity thereto (e.g., having 1 or 2 conservative amino acid substitutions). These multispecific constructs can be used therapeutically as treatments for autoimmune disorders and diseases, including but not limited to, those shown in Table 1.

In particular embodiments, for example, a CD68 binding arm of the multispecific construct comprises CDRs selected from the anti-CD68 monoclonal antibodies Y1/82A and C68/684, wherein the binding domain of the CD68 binding arm comprises a CDRVL1 comprising the amino acid sequence SASSSVSYMY (SEQ ID NO:50) or RASKSVSTSGYSYMH (SEQ ID NO:56); a CDRVL2 comprising the amino acid sequence RTSNLAS (SEQ ID NO:51) or LASNLES (SEQ ID NO:57); a CDRVL3 comprising the amino acid sequence QQYHFFPPT (SEQ ID NO:52) or QHSRELPYT (SEQ ID NO:58); a CDRVH1 comprising the amino acid sequence GYTFINY (SEQ ID NO:53) or GYTFTNY (SEQ ID NO:59); a CDRVH2 comprising the amino acid sequence LPGSHT (SEQ ID NO:54) or YPGDGD (SEQ ID NO:60); and a CDRVH3 comprising the amino acid sequence GGTRASFYYFDY (SEQ ID NO:55) or EPDDYDGYYYFDY (SEQ ID NO:61). These multispecific constructs can be used therapeutically as treatments for autoimmune disorders and diseases, including but not limited to, those shown in Table 1.

In one non-limiting embodiment the present disclosure is directed to a bispecific construct comprising a CD20 binding arm and a CD68 binding arm in which the CD20 binding arm comprises the CDRs of the anti-CD20 monoclonal antibody Rituximab, and the CD68 binding arm comprises the CDRs of the anti-CD68 monoclonal antibody Y1/82A. More particularly, the CD20 binding arm comprises a CDRVL1 comprising the amino acid sequence RASSSVSYIHW (SEQ ID NO:1) or an amino acid sequence having at least 80% or at least 90% identity thereto (e.g., having 1 or 2 conservative amino acid substitutions); a CDRVL2 comprising the amino acid sequence ATSNLAS (SEQ ID NO:2), or an amino acid sequence having at least 80% or at least 90% identity thereto (e.g., having 1 or 2 conservative amino acid substitutions); a CDRVL3 comprising the amino acid sequence QQWTSNPPT (SEQ ID NO:3), or an amino acid sequence having at least 80% or at least 90% identity thereto (e.g., having 1 or 2 conservative amino acid substitutions); a CDRVH1 comprising the amino acid sequence SYNMH (SEQ ID NO:4), or an amino acid sequence having at least 80% or at least 90% identity thereto (e.g., having 1 or 2 conservative amino acid substitutions); a CDRVH2 comprising the amino acid sequence AIYPGNGDTSYNQKFKG (SEQ ID NO:5), or an amino acid sequence having at least 80% or at least 90% identity thereto (e.g., having 1 or 2 conservative amino acid substitutions); and a CDRVH3 comprising the amino acid sequence STYYGGDWYFNV (SEQ ID NO:6), or an amino acid sequence having at least 80% or at least 90% identity thereto (e.g., having 1 or 2 conservative amino acid substitutions), and the CD68 binding arm comprises a CDRVL1 comprising the amino acid sequence SASSSVSYMY (SEQ ID NO:50), or an amino acid sequence having at least 80% or at least 90% identity thereto (e.g., having 1 or 2 conservative amino acid substitutions); a CDRVL2 comprising the amino acid sequence RTSNLAS (SEQ ID NO:51), or an amino acid sequence having at least 80% or at least 90% identity thereto (e.g., having 1 or 2 conservative amino acid substitutions); a CDRVL3 comprising the amino acid sequence QQYHFFPPT (SEQ ID NO:52), or an amino acid sequence having at least 80% or at least 90% identity thereto (e.g., having 1 or 2 conservative amino acid substitutions); a CDRVH1 comprising the amino acid sequence GYTFINY (SEQ ID NO:53), or an amino acid sequence having at least 80% or at least 90% identity thereto (e.g., having 1 or 2 conservative amino acid substitutions); a CDRVH2 comprising the amino acid sequence LPGSHT (SEQ ID NO:54), or an amino acid sequence having at least 80% or at least 90% identity thereto (e.g., having 1 or 2 conservative amino acid substitutions); and a CDRVH3 comprising the amino acid sequence GGTRASFYYFDY (SEQ ID NO:55), or an amino acid sequence having at least 80% or at least 90% identity thereto (e.g., having 1 or 2 conservative amino acid substitutions). These multispecific constructs can be used therapeutically as treatments for autoimmune disorders and diseases, including but not limited to, those shown in Table 1.

In one non-limiting embodiment, the present disclosure is directed to a bispecific construct comprising a CD20 binding arm and a CD68 binding arm in which the CD20 binding arm comprises the CDRs of the anti-CD20 monoclonal antibody Rituximab, and the CD68 binding arm comprises the CDRs of the anti-CD68 monoclonal antibody C68/684. More particularly, the CD20 binding arm comprises a CDRVL1 comprising the amino acid sequence RASSSVSYIHW (SEQ ID NO:1) or an amino acid sequence having at least 80% or at least 90% identity thereto (e.g., having 1 or 2 conservative amino acid substitutions); a CDRVL2 comprising the amino acid sequence ATSNLAS (SEQ ID NO:2), or an amino acid sequence having at least 80% or at least 90% identity thereto (e.g., having 1 or 2 conservative amino acid substitutions); a CDRVL3 comprising the amino acid sequence QQWTSNPPT (SEQ ID NO:3), or an amino acid sequence having at least 80% or at least 90% identity thereto (e.g., having 1 or 2 conservative amino acid substitutions); a CDRVH1 comprising the amino acid sequence SYNMH (SEQ ID NO:4), or an amino acid sequence having at least 80% or at least 90% identity thereto (e.g., having 1 or 2 conservative amino acid substitutions); a CDRVH2 comprising the amino acid sequence AIYPGNGDTSYNQKFKG (SEQ ID NO:5), or an amino acid sequence having at least 80% or at least 90% identity thereto (e.g., having 1 or 2 conservative amino acid substitutions); and a CDRVH3 comprising the amino acid sequence STYYGGDWYFNV (SEQ ID NO:6), or an amino acid sequence having at least 80% or at least 90% identity thereto (e.g., having 1 or 2 conservative amino acid substitutions), and the CD68 binding arm comprises a CDRVL1 comprising the amino acid sequence RASKSVSTSGYSYMH (SEQ ID NO:56), or an amino acid sequence having at least 80% or at least 90% identity thereto (e.g., having 1 or 2 conservative amino acid substitutions); a CDRVL2 comprising the amino acid sequence LASNLES (SEQ ID NO:57), or an amino acid sequence having at least 80% or at least 90% identity thereto (e.g., having 1 or 2 conservative amino acid substitutions); a CDRVL3 comprising the amino acid sequence QHSRELPYT (SEQ ID NO:58), or an amino acid sequence having at least 80% or at least 90% identity thereto (e.g., having 1 or 2 conservative amino acid substitutions); a CDRVH1 comprising the amino acid sequence GYTFTNY (SEQ ID NO:59), or an amino acid sequence having at least 80% or at least 90% identity thereto (e.g., having 1 or 2 conservative amino acid substitutions); a CDRVH2 comprising the amino acid sequence YPGDGD (SEQ ID NO:60), or an amino acid sequence having at least 80% or at least 90% identity thereto (e.g., having 1 or 2 conservative amino acid substitutions); and a CDRVH3 comprising the amino acid sequence EPDDYDGYYYFDY (SEQ ID NO:61), or an amino acid sequence having at least 80% or at least 90% identity thereto (e.g., having 1 or 2 conservative amino acid substitutions). These multispecific constructs can be used therapeutically as treatments for autoimmune disorders and diseases, including but not limited to, those shown in Table 1.

In one non-limiting embodiment, the present disclosure is directed to a bispecific construct comprising a CD20 binding arm and a CD68 binding arm in which the CD20 binding arm comprises the CDRs of the anti-CD20 monoclonal antibody Rituximab, and the CD68 binding arm comprises the CDRs of an anti-CD68 monoclonal antibody. More particularly, the CD20 binding arm comprises a CDRVL1 comprising the amino acid sequence RASSSVSYIHW (SEQ ID NO:1) or an amino acid sequence having at least 80% or at least 90% identity thereto; a CDRVL2 comprising the amino acid sequence ATSNLAS (SEQ ID NO:2), or an amino acid sequence having at least 80% or at least 90% identity thereto; a CDRVL3 comprising the amino acid sequence QQWTSNPPT (SEQ ID NO:3), or an amino acid sequence having at least 80% or at least 90% identity thereto; a CDRVH1 comprising the amino acid sequence SYNMH (SEQ ID NO:4), or an amino acid sequence having at least 80% or at least 90% identity thereto; a CDRVH2 comprising the amino acid sequence AIYPGNGDTSYNQKFKG (SEQ ID NO:5), or an amino acid sequence having at least 80% or at least 90% identity thereto; and a CDRVH3 comprising the amino acid sequence STYYGGDWYFNV (SEQ ID NO:6), or an amino acid sequence having at least 80% or at least 90% identity thereto, and the CD68 binding arm comprises CDRs selected from the anti-CD68 monoclonal antibodies Y1/82A and C68/684, wherein the binding domain of the first binding arm comprises a CDRVL1 comprising the amino acid sequence SASSSVSYMY (SEQ ID NO:50) or RASKSVSTSGYSYMH (SEQ ID NO:56); a CDRVL2 comprising the amino acid sequence RTSNLAS (SEQ ID NO:51) or LASNLES (SEQ ID NO:57); a CDRVL3 comprising the amino acid sequence QQYHFFPPT (SEQ ID NO:52) or QHSRELPYT (SEQ ID NO:58); a CDRVH1 comprising the amino acid sequence GYTFINY (SEQ ID NO:53) or GYTFTNY (SEQ ID NO:59); a CDRVH2 comprising the amino acid sequence LPGSHT (SEQ ID NO:54) or YPGDGD (SEQ ID NO:60); and a CDRVH3 comprising the amino acid sequence GGTRASFYYFDY (SEQ ID NO:55) or EPDDYDGYYYFDY (SEQ ID NO:61). These multispecific constructs can be used therapeutically as treatments for autoimmune disorders and diseases, including but not limited to, those shown in Table 1.

Table 5 shows CDR sequences of various anti-CD68 mAbs described above.

TABLE 5
CDR sequences of various anti-CD68 mAbs

| Amino acid sequence | SEQ ID NO: | Source antibody | CDR Descriptor |
|---|---|---|---|
| SASSSVSYMY | SEQ ID NO: 50 | Y1/82A | CDRVL1 |
| RTSNLAS | SEQ ID NO: 51 | Y1/82A | CDRVL2 |
| QQYHFFPPT | SEQ ID NO: 52 | Y1/82A | CDRVL3 |
| GYTFINY | SEQ ID NO: 53 | Y1/82A | CDRVH1 |
| LPGSHT | SEQ ID NO: 54 | Y1/82A | CDRVH2 |
| GGTRASFYYFDY | SEQ ID NO: 55 | Y1/82A | CDRVH3 |
| RASKSVSTSGYSYMH | SEQ ID NO: 56 | C68/684 | CDRVL1 |
| LASNLES | SEQ ID NO: 57 | C68/684 | CDRVL2 |
| QHSRELPYT | SEQ ID NO: 58 | C68/684 | CDRVL3 |
| GYTFTNY | SEQ ID NO: 59 | C68/684 | CDRVH1 |
| YPGDGD | SEQ ID NO: 60 | C68/684 | CDRVH2 |
| EPDDYDGYYYFDY | SEQ ID NO: 61 | C68/684 | CDRVH3 |

TABLE 6
Exemplary Anti-CD68 monoclonal antibodies and sources

| Identifier | Commercial source |
|---|---|
| Y1/82A | Invitrogen |
| CD68/684 | Abcam |
| KP1 | Invitrogen |
| FA-11 | Invitrogen |
| EPR20545 | Abcam |
| EPR23917-164 | Abcam |
| EPR24100-133 | Abcam |
| EPR24100-8 | Abcam |
| SP-251 | Abcam |
| ED1 | Invitrogen |
| 3F7D3 | Abcam |
| PG-M1 | Invitrogen |
| CD68-2501 | Abcam |
| LAMP4-1830 | Abcam |
| 815CU17 | Invitrogen |
| 514H12 | Invitrogen |
| Ki-M7 | Invitrogen |
| 298807 | Invitrogen |
| Zr302 | Invitrogen |
| 3A9A7 | Invitrogen |
| OTI8A4 | Invitrogen |
| UMAB150 | Invitrogen |
| OTI3b6 | Invitrogen |
| SN07-27 | Invitrogen |
| LAMP4-824 | NeoBiotechnologies |
| C68-2908R | NeoBiotechnologies |

In at least some embodiments, the present disclosure is directed to a multispecific construct (e.g., a bispecific construct) comprising a CD20 binding arm comprising the CDRs of at least one of the anti-CD20 monoclonal antibodies selected from Obinutuzumab, Ocrelizumab, Ocaratuzumab, Ofatumumab, Rituximab, Tositumomab, Ublituximab, Ibritumomab, Veltuzumab, PRO131921, 7D8, 2F2, 11B8, and 2C6, and a CD68 binding arm comprising the CDRs of at least one of the anti-CD68 monoclonal antibodies selected from Y1/82A; CD68/684; KP1; FA-11; EPR20545; EPR23917-164; EPR24100-133; EPR24100-8; SP-251; ED1; 3F7D3; PG-M1; CD68-2501; LAMP4-1830; 815CU17; 514H12; Ki-M7; 298807; Zr302; 3A9A7; OTI8A; OTI3b6; UMAB150;SN07-27; LAMP4-824; and C68-2908R. As noted above, any one of these constructs can be used therapeutically as a treatment for autoimmune disorders and diseases, including but not limited to, those shown in Table 1, such as SLE, SLE flare-ups, lupus nephritis, acute or chronic Graft versus Host Disease (GvHD), Rheumatoid Arthritis (RA), Pemphigus, e.g., Pemphigus vulgaris (PVu), Pemphigus vegetans (PVe), Pemphigus erythematosus (PE), and Pemphigus foliaceus (PF), Chronic Lymphocytic Leukemia (CLL), Granulomatosis with Polyangiitis (GPA), Microscopic Polyangiitis (MPA), Multiple Sclerosis (MS), Primary Progressive Multiple Sclerosis (PPMS), Relapsing Multiple Sclerosis (RMS), Anti-neutrophil Cytoplasmic Antibody (ANCA)-associated vasculitis, Non-Hodgkin's Lymphoma (NHL), Rassmussen's encephalitis, Immune Thrombocytopenic Purpura (ITP), Follicular Lymphoma Leukemia (FLL), immune conditions treatable by B cell depletion therapies (BCDT), Inflammatory Bowel Disease (IBD), Ulcerative colitis (UC), Crohn's disease (CD), Pediatric Acute-onset Neuropsychiatric Syndrome (PANS), and Pediatric Autoimmune Neuropsychiatric Disorder Associated with Streptoccocal Infections (PANDAS).

In at least some embodiments, a CD20 binding arm of the multispecific construct comprises the CDRs of the anti-CD20 monoclonal antibody rituximab, and a CD68 binding arm of the multispecific construct comprises the CDRs of one of the anti-CD68 monoclonal antibodies selected from Y1/82A and CD68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In at least some embodiments, a CD20 binding arm of the multispecific construct comprises the CDRs of the anti-CD20 monoclonal antibody obinutuzumab, and a CD68 binding arm of the multispecific construct comprises the CDRs of one of the anti-CD68 monoclonal antibodies selected from Y1/82A and CD68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In at least some embodiments, a CD20 binding arm of the multispecific construct comprises the CDRs of the anti-CD20 monoclonal antibody ofatumumab, and a CD68 binding arm of the multispecific construct comprises the CDRs of one of the anti-CD68 monoclonal antibodies selected from Y1/82A and CD68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In at least some embodiments, a CD20 binding arm of the multispecific construct comprises the CDRs of the anti-CD20 monoclonal antibody ocrelizumab, and a CD68 binding arm of the multispecific construct comprises the CDRs of one of the anti-CD68 monoclonal antibodies selected from Y1/82A and CD68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In at least some embodiments, a CD20 binding arm of the multispecific construct comprises the CDRs of the anti-CD20 monoclonal antibody Tositumomab, and a CD68 binding arm of the multispecific construct comprises the CDRs of one of the anti-CD68 monoclonal antibodies selected from Y1/82A and CD68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In at least some embodiments, a CD20 binding arm of the multispecific construct comprises the CDRs of the anti-CD20 monoclonal antibody Ublituximab, and a CD68 binding arm of the multispecific construct comprises the CDRs of one of the anti-CD68 monoclonal antibodies selected from Y1/82A and CD68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In at least some embodiments, a CD20 binding arm of the multispecific construct comprises the CDRs of the anti-CD20 monoclonal antibody Ibritumomab, and a CD68 binding arm of the multispecific construct comprises the CDRs of one of the anti-CD68 monoclonal antibodies selected from Y1/82A and CD68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In at least some embodiments, a CD20 binding arm of the multispecific construct comprises the CDRs of the anti-CD20 monoclonal antibody veltuzumab, and a CD68 binding arm of the multispecific construct comprises the CDRs of one of the anti-CD68 monoclonal antibodies selected from Y1/82A and CD68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In at least some embodiments, a CD20 binding arm of the multispecific construct comprises the CDRs of the anti-CD20 monoclonal antibody ocaratuzumab, and a CD68 binding arm of the multispecific construct comprises the CDRs of one of the anti-CD68 monoclonal antibodies selected from Y1/82A and CD68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In at least some embodiments, a CD20 binding arm of the multispecific construct comprises the CDRs of the anti-CD20 monoclonal antibody PRO131921, and a CD68 binding arm of the multispecific construct comprises the CDRs of one of the anti-CD68 monoclonal antibodies selected from Y1/82A and CD68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In at least some embodiments, a CD20 binding arm of the multispecific construct comprises the CDRs of the anti-CD20 monoclonal antibody 7D8, and a CD68 binding arm of the multispecific construct comprises the CDRs of one of the anti-CD68 monoclonal antibodies selected from Y1/82A and CD68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In at least some embodiments, a CD20 binding arm of the multispecific construct comprises the CDRs of the anti-CD20 monoclonal antibody 2F2, and a CD68 binding arm of the multispecific construct comprises the CDRs of one of the anti-CD68 monoclonal antibodies selected from Y1/82A and CD68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In at least some embodiments, a CD20 binding arm of the multispecific construct comprises the CDRs of the anti-CD20 monoclonal antibody 11B8, and a CD68 binding arm of the multispecific construct comprises the CDRs of one of the anti-CD68 monoclonal antibodies selected from Y1/82A and CD68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In at least some embodiments, a CD20 binding arm of the multispecific construct comprises the CDRs of the anti-CD20 monoclonal antibody 2C6, and a CD68 binding arm of the multispecific construct comprises the CDRs of one of the anti-CD68 monoclonal antibodies selected from Y1/82A and CD68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In one embodiment, the multispecific construct of the present disclosure comprises a CD20 binding arm which comprises the three VL CDRs and three VH CDRs of Obinutuzumab, and a CD68 binding arm which comprises the three VL CDRs and three VH CDRs of Y1/82A or the three VL CDRs and three VH CDRs of C68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In one embodiment, the multispecific construct of the present disclosure comprises a CD20 binding arm which comprises the three VL CDRs and three VH CDRs of Ocrelizumab, and a CD68 binding arm which comprises the three VL CDRs and three VH CDRs of Y1/82A, or the three VL CDRs and three VH CDRs of C68/684. In one embodiment, the multispecific construct of the present disclosure comprises a CD20 binding arm which comprises the three VL CDRs and three VH CDRs of Ocrelizumab, and a CD68 binding arm which comprises the three VL CDRs and three VH CDRs of Y1/82A or the three VL CDRs and three VH CDRs of C68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In one embodiment, the multispecific construct of the present disclosure comprises a CD20 binding arm which comprises the three VL CDRs and three VH CDRs of Ofatumumab, and a CD68 binding arm which comprises the three VL CDRs and three VH CDRs of Y1/82A or the three VL CDRs and three VH CDRs of C68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In one embodiment, the multispecific construct of the present disclosure comprises a CD20 binding arm which comprises the three VL CDRs and three VH CDRs of Rituximab, and a CD68 binding arm which comprises the three VL CDRs and three VH CDRs of Y1/82A or the three VL CDRs and three VH CDRs of C68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In one embodiment, the multispecific construct of the present disclosure comprises a CD20 binding arm which comprises the three VL CDRs and three VH CDRs of Tositumomab, and a CD68 binding arm which comprises the three VL CDRs and three VH CDRs of Y1/82A or the three VL CDRs and three VH CDRs of C68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In one embodiment, the multispecific construct of the present disclosure comprises a CD20 binding arm which comprises the three VL CDRs and three VH CDRs of Ublituximab, and a CD68 binding arm which comprises the three VL CDRs and three VH CDRs of Y1/82A or the three VL CDRs and three VH CDRs of C68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In one embodiment, the multispecific construct of the present disclosure comprises a CD20 binding arm which comprises the three VL CDRs and three VH CDRs of Ibritumomab, and a CD68 binding arm which comprises the three VL CDRs and three VH CDRs of Y1/82A or the three VL CDRs and three VH CDRs of C68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In one embodiment, the multispecific construct of the present disclosure comprises a CD20 binding arm which comprises the three VL CDRs and three VH CDRs of Veltuzumab, and a CD68 binding arm which comprises the three VL CDRs and three VH CDRs of Y1/82A or the three VL CDRs and three VH CDRs of C68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In one embodiment, the multispecific construct of the present disclosure comprises a CD20 binding arm which comprises the three VL CDRs and three VH CDRs of PRO131921, and a CD68 binding arm which comprises the three VL CDRs and three VH CDRs of Y1/82A or the three VL CDRs and three VH CDRs of C68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In one embodiment, the multispecific construct of the present disclosure comprises a CD20 binding arm which comprises the three VL CDRs and three VH CDRs of 7D8, and a CD68 binding arm which comprises the three VL CDRs and three VH CDRs of Y1/82A or the three VL CDRs and three VH CDRs of C68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In one embodiment, the multispecific construct of the present disclosure comprises a CD20 binding arm which comprises the three VL CDRs and three VH CDRs of 2F2, and a CD68 binding arm which comprises the three VL CDRs and three VH CDRs of Y1/82A or the three VL CDRs and three VH CDRs of C68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In one embodiment, the multispecific construct of the present disclosure comprises a CD20 binding arm which comprises the three VL CDRs and three VH CDRs of 11B8, and a CD68 binding arm which comprises the three VL CDRs and three VH CDRs of Y1/82A or the three VL CDRs and three VH CDRs of C68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

In one embodiment, the multispecific construct of the present disclosure comprises a CD20 binding arm which comprises the three VL CDRs and three VH CDRs of 2C6, and a CD68 binding arm which comprises the three VL CDRs and three VH CDRs of Y1/82A, or the three VL CDRs and three VH CDRs of C68/684. Any one of these constructs can be used therapeutically as a treatment for autoimmune disorders and diseases, including but not limited to, SLE, SLE flare-ups, lupus nephritis, acute or chronic GvHD, RA, Pemphigus, PVu, PVe, PE, PF, CLL, GPA, MPA, MS, PPMS, RMS, ANCA-associated vasculitis, NHL, Rassmussen's encephalitis, ITP, FLL, immune conditions treatable by BCDT, IBD, UC, CD, PANS, and PANDAS.

More particularly, the present disclosure is directed to the following bispecific constructs and their uses in treating autoimmune disorders and diseases.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb Y1/82A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb CD68/684. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb KP1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb FA-11. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb EPR20545. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb EPR23917-164. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb EPR24100-133. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb EPR24100-8. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb SP-251. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb ED1. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb 3F7D3. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb PG-M1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb CD68-2501. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb LAMP4-1830. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb 815CU17. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb 514H12. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb Ki-M7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb 298807. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb Zr302. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb 3A9A7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb OTI8A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb OTI3b6. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb UMAB150. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb SN07-27. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb LAMP4-824. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Rituximab; and a CD68 binding arm comprising the 3 VH CDRs and 3 VL CDRs of the anti-CD68 mAb C68-2908R. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb Y1/82A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb CD68/684. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb KP1. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb FA-11. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb EPR20545. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb EPR23917-164. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb EPR24100-133. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb EPR24100-8. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb SP-251. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb ED1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb 3F7D3. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb PG-M1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb CD68-2501. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb LAMP4-1830. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb 815CU17. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb 514H12. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb Ki-M7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb 298807. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb Zr302. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb 3A9A7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb OTI8A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb OTI3b6. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb UMAB150. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb SN07-27. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb LAMP4-824. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocaratuzumab; and a CD68 binding arm comprising the 3 VH CDRs and 3 VL CDRs of the anti-CD68 mAb C68-2908R. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb Y1/82A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb CD68/684. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb KP1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb FA-11. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb EPR20545. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb EPR23917-164. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb EPR24100-133. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb EPR24100-8. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb SP-251. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb ED1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb 3F7D3. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb PG-M1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb CD68-2501. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb LAMP4-1830. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb 815CU17. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb 514H12. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb Ki-M7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb 298807. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb Zr302. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb 3A9A7. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb OTI8A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb OTI3b6. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb UMAB150. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb SN07-27. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb LAMP4-824. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ocrelizumab; and a CD68 binding arm comprising the 3 VH CDRs and 3 VL CDRs of the anti-CD68 mAb C68-2908R. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb Y1/82A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb CD68/684. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb KP1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb FA-11. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb EPR20545. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb EPR23917-164. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb EPR24100-133. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb EPR24100-8. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb SP-251. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb ED1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb 3F7D3. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb PG-M1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb CD68-2501. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb LAMP4-1830. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb 815CU17. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb 514H12. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb Ki-M7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb 298807. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb Zr302. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb 3A9A7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb OTI8A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb OTI3b6. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb UMAB150. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb SN07-27. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb LAMP4-824. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ofatumumab; and a CD68 binding arm comprising the 3 VH CDRs and 3 VL CDRs of the anti-CD68 mAb C68-2908R. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb Y1/82A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb CD68/684. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb KP1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb FA-11. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb EPR20545. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb EPR23917-164. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb EPR24100-133. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb EPR24100-8. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb SP-251. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb ED1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb 3F7D3. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb PG-M1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb CD68-2501. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb LAMP4-1830. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb 815CU17. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb 514H12. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb Ki-M7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb 298807. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb Zr302. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb 3A9A7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb OTI8A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb OTI3b6. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb UMAB150. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb SN07-27. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb LAMP4-824. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Tositumomab; and a CD68 binding arm comprising the 3 VH CDRs and 3 VL CDRs of the anti-CD68 mAb C68-2908R. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb Y1/82A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb CD68/684. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb KP1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb FA-11. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb EPR20545. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb EPR23917-164. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb EPR24100-133. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb EPR24100-8. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb SP-251. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb ED1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb 3F7D3. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb PG-M1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb CD68-2501. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb LAMP4-1830. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb 815CU17. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb 514H12. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb Ki-M7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb 298807. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb Zr302. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb 3A9A7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb OTI8A. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb OTI3b6. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb UMAB150. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb SN07-27. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb LAMP4-824. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ublituximab; and a CD68 binding arm comprising the 3 VH CDRs and 3 VL CDRs of the anti-CD68 mAb C68-2908R. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb Y1/82A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb CD68/684. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb KP1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb FA-11. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb EPR20545. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb EPR23917-164. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb EPR24100-133. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb EPR24100-8. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb SP-251. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb ED1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb 3F7D3. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb PG-M1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb CD68-2501. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb LAMP4-1830. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb 815CU17. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb 514H12. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb Ki-M7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb 298807. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb Zr302. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb 3A9A7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb OTI8A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb OTI3b6. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb UMAB150. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb SN07-27. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb LAMP4-824. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Ibritumomab; and a CD68 binding arm comprising the 3 VH CDRs and 3 VL CDRs of the anti-CD68 mAb C68-2908R. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb Y1/82A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb CD68/684. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb KP1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb FA-11. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb EPR20545. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb EPR23917-164. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb EPR24100-133. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb EPR24100-8. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb SP-251. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb ED1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb 3F7D3. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb PG-M1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb CD68-2501. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb LAMP4-1830. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb 815CU17. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb 514H12. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb Ki-M7. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb 298807. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb Zr302. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb 3A9A7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb OTI8A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb OTI3b6. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb UMAB150. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb SN07-27. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb LAMP4-824. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb Veltuzumab; and a CD68 binding arm comprising the 3 VH CDRs and 3 VL CDRs of the anti-CD68 mAb C68-2908R. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb Y1/82A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb CD68/684. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb KP1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb FA-11. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb EPR20545. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb EPR23917-164. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb EPR24100-133. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb EPR24100-8. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb SP-251. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb ED1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb 3F7D3. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb PG-M1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb CD68-2501. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb LAMP4-1830. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb 815CU17. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb 514H12. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb Ki-M7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb 298807. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb Zr302. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb 3A9A7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb OTI8A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb OTI3b6. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb UMAB150. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb SN07-27. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb LAMP4-824. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb PRO131921; and a CD68 binding arm comprising the 3 VH CDRs and 3 VL CDRs of the anti-CD68 mAb C68-2908R. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb Y1/82A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb CD68/684. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb KP1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb FA-11. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb EPR20545. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb EPR23917-164. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb EPR24100-133. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb EPR24100-8. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb SP-251. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb ED1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb 3F7D3. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb PG-M1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb CD68-2501. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb LAMP4-1830. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb 815CU17. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb 514H12. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb Ki-M7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb 298807. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb Zr302. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb 3A9A7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb OTI8A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb OTI3b6. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb UMAB150. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb SN07-27. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb LAMP4-824. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 7D8; and a CD68 binding arm comprising the 3 VH CDRs and 3 VL CDRs of the anti-CD68 mAb C68-2908R. The bispecific construct can be used therapeutically as a treatment for autoimmune disorders and diseases, including but not limited to, those shown in Table 1

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb Y1/82A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb CD68/684. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb KP1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb FA-11. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb EPR20545. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb EPR23917-164. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb EPR24100-133. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb EPR24100-8. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb SP-251. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb ED1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb 3F7D3. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb PG-M1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb CD68-2501. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb LAMP4-1830. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb 815CU17. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb 514H12. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb Ki-M7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb 298807. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb Zr302. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb 3A9A7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb OTI8A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb OTI3b6. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb UMAB150. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb SN07-27. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb LAMP4-824. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2F2; and a CD68 binding arm comprising the 3 VH CDRs and 3 VL CDRs of the anti-CD68 mAb C68-2908R. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb Y1/82A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb CD68/684. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb KP1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb FA-11. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb EPR20545. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb EPR23917-164. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb EPR24100-133. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb EPR24100-8. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb SP-251. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb ED1. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb 3F7D3. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb PG-M1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb CD68-2501. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb LAMP4-1830. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb 815CU17. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb 514H12. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb Ki-M7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb 298807. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb Zr302. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb 3A9A7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb OTI8A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb OTI3b6. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb UMAB150. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb SN07-27. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb LAMP4-824. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 11B8; and a CD68 binding arm comprising the 3 VH CDRs and 3 VL CDRs of the anti-CD68 mAb C68-2908R. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb Y1/82A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb CD68/684. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb KP1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb FA-11. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb EPR20545. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb EPR23917-164. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb EPR24100-133. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb EPR24100-8. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb SP-251. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb ED1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb 3F7D3. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb PG-M1. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb CD68-2501. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb LAMP4-1830. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb 815CU17. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb 514H12. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb Ki-M7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb 298807. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb Zr302. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb 3A9A7. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb OTI8A. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb OTI3b6. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb UMAB150. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb SN07-27. The bispecific construct can be used therapeutically as a treatment for autoimmune disorders and diseases, including but not limited to, those shown Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb LAMP4-824. The bispecific construct can be used therapeutically as a treatment for autoimmune disorders and diseases, including but not limited to, those shown in Table 1.

In one embodiment, the present disclosure is directed to a bispecific construct comprising a CD20 binding arm comprising the 3 VH CDRs and 3 VL CDRs of the anti-CD20 mAb 2C6; and a CD68 binding arm comprising the 3 VH CDRs and 3 VL CDRs of the anti-CD68 mAb C68-2908R. The bispecific construct can be used therapeutically as a treatment for autoimmune disorders and diseases, including but not limited to, those shown in Table 1.

In certain embodiments, the present disclosure is directed to a multispecific construct, comprising an anti-B cell binding arm that specifically binds to a B-cell-specific cell surface marker, and a second binding arm that specifically binds to cluster of differentiation (CD) 68 (CD68) protein, and optionally at least one additional binding arm, wherein the multispecific construct binds specifically to naïve B cells which express transcription factor ARID3a. The B cell-specific cell surface marker may be selected from the group consisting of CD20, CD19, CD21, CD24, and CD38. The anti-B cell binding arm of the multispecific construct may comprise a first complementarity determining region (CDR) H₁ (CDRH₁), a first CDRH₂, a first CDRH₃, a first CDRL₁, a first CDRL₂, and a first CDRL₃ which are obtained from an anti-CD20 antibody, and the second binding arm of the multispecific construct may comprise a second CDRH₁, a second CDRH₂, a second CDRH₃, a second CDRL₁, a second CDRL₂, and a second CDRL₃ which are obtained from an anti-CD68 antibody. The anti-CD20 antibody of the multispecific constructs described above may be selected from the group consisting of rituximab, obinutuzumab, ofatumumab, ocrelizumab, veltuzumab, ocaratuzumab, tositumomab, ublituximab, ibritumomab, PRO131921, 7D8, 2F2, 11B8, and 2C6. The anti-CD68 antibody of the multispecific constructs described above may be selected from the group consisting of Y1/82A; CD68/684; KP1; FA-11; EPR20545; EPR23917-164; EPR24100-133; EPR24100-8; SP-251; ED1; 3F7D3; PG-M1; CD68-2501; LAMP4-1830; 815CU17; 514H12; Ki-M7; 298807; Zr302; 3A9A7; OTI8A; OTI3b6; UMAB150; SN07-27; LAMP4-824; and C68-2908R. The anti-B cell binding arm of the multispecific constructs described above may comprise a heavy chain variable sequence and a light chain variable sequence of an anti-CD20 antibody, and the second binding arm of the multispecific constructs described above may comprise a heavy chain variable sequence and a light chain variable sequence of an anti-CD68 antibody. The anti-CD20 antibody of the multispecific constructs described above may be selected from the group consisting of rituximab, obinutuzumab, ofatumumab, ocrelizumab, veltuzumab, ocaratuzumab, tositumomab, ublituximab, ibritumomab, PRO131921, 7D8, 2F2, 11B8, and 2C6. The anti-CD68 antibody of the multispecific constructs described above may be selected from the group consisting of Y1/82A; CD68/684; KP1; FA-11; EPR20545; EPR23917-164; EPR24100-133; EPR24100-8; SP-251; ED1; 3F7D3; PG-M1; CD68-2501; LAMP4-1830; 815CU17; 514H12; Ki-M7; 298807; Zr302; 3A9A7; OTI8A; OTI3b6; UMAB150; SN07-27; LAMP4-824; and C68-2908R. The anti-B cell binding arm and second binding arm of the multispecific constructs described above may be chimeric, human, partially humanized, fully humanized, or semi-synthetic. The multispecific constructs described above may be selected from the group consisting of a full-length antibody, a F(ab′)₂, a chemically-linked F(ab′)2, a tandem scFv, a diabody (Db), a single chain diabody (scDb), a tandem diabody, a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, and a heteroconjugate antibody. The multispecific constructs described above may comprise a chimeric antigen receptor (CAR) T-cell. The multispecific constructs described above may be bispecific constructs. In certain embodiments, the present disclosure is directed to a method of treating an autoimmune disorder and/or disease in a subject in need of such therapy, comprising administering to the subject one of more of the multispecific constructs described above, wherein the autoimmune disorder and/or disease is selected from the group consisting of Systemic lupus erythematosus (SLE), SLE flare-ups, lupus nephritis (LN), acute Graft versus Host Disease (GvHD), chronic GvHD, Rheumatoid Arthritis (RA), Pemphigus, e.g., Pemphigus vulgaris (PVu), Pemphigus vegetans (PVe), Pemphigus erythematosus (PE), and Pemphigus foliaceus (PF), Chronic Lymphocytic Leukemia (CLL), Granulomatosis with Polyangiitis (GPA), Microscopic Polyangiitis (MPA), Multiple Sclerosis (MS), Primary Progressive Multiple Sclerosis (PPMS), Relapsing Multiple Sclerosis (RMS), Anti-neutrophil Cytoplasmic Antibody (ANCA)-associated vasculitis, Non-Hodgkin's Lymphoma (NHL), Rassmussen's encephalitis, Immune Thrombocytopenic Purpura (ITP), Follicular Lymphoma Leukemia (FLL), and other immune conditions treatable by B cell depletion therapies (BCDT), Inflammatory Bowel Disease (IBD), Ulcerative colitis (UC), Crohn's disease (CD), Pediatric Acute-onset Neuropsychiatric Syndrome (PANS), and Pediatric Autoimmune Neuropsychiatric Disorder Associated with Streptoccocal Infections (PANDAS). In certain embodiments, the present disclosure is directed to a method of inhibiting autoantibody production in a subject in need of such therapy, comprising administering to the subject one or more of the multispecific constructs described above. In certain embodiments, the present disclosure is directed to a method of inhibiting autoantibody production in naïve B cells which express CD68, comprising administering to the naïve B cells one or more of the multispecific constructs described above. In certain embodiments, the present disclosure is directed to an isolated polynucleotide, comprising a nucleotide sequence encoding at least one of the multispecific constructs described above. In certain embodiments, the present disclosure is directed to a vector comprising the polynucleotide described above. In certain embodiments, the present disclosure is directed to a host cell comprising the polynucleotide described above. In certain embodiments, the present disclosure is directed to a method of treating a lupus flare-up in a subject who has Systemic lupus erythematosus (SLE), comprising: (a) analyzing a blood sample from the subject to determine a test quantity of CD68⁺ naïve B cells in the blood sample; (b) comparing the test quantity of CD68⁺ naïve B cells to a control quantity of CD68⁺ naïve B cells obtained from a cohort of healthy subjects or to a predictive quantity CD68⁺ naïve B cells predictive of a flare-up; (c) predicting that the subject will experience a lupus flare-up when (1) the test quantity of CD68⁺ naïve B cells (1) exceeds the control quantity of CD68⁺ naïve B cells, or (2) is equal to or exceeds the predictive quantity CD68⁺ naïve B cells; and (d) treating the subject with one or more of the multispecific constructs described above when the subject is predicted to experience a lupus flare-up. In certain embodiments of the method of treating a lupus flare-up, the CD68⁺ naïve B cells are determined to be positive for CD20 and IgD, and negative for CD27. In certain embodiments of the method of treating a lupus flare-up, the CD68⁺ naïve B cells are positive for transcription factor ARID3a. In certain embodiments of the method of treating a lupus flare-up, the CD68⁺ naïve B cells produce autoantibodies.

While the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the embodiments described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of methods and procedures as well as of the principles and conceptual aspects of the present disclosure.

Changes may be made in the formulation of the various compositions described herein, the methods described herein or in the steps or the sequence of steps of the methods described herein without departing from the spirit and scope of the present disclosure. Further, while various embodiments of the present disclosure have been described in exemplary claims herein below, it is not intended that the present disclosure be limited to these particular exemplary claims.

1. A multispecific construct, comprising an anti-B cell binding arm that specifically binds to a B-cell-specific cell surface marker, and a second binding arm that specifically binds to cluster of differentiation (CD) 68 (CD68) protein, and optionally at least one additional binding arm, wherein the multispecific construct binds specifically to naïve B cells which express transcription factor ARID3a.

2. The multispecific construct of claim 1, wherein the B cell-specific cell surface marker is selected from the group consisting of CD20, CD19, CD21, CD24, and CD38.

3. The multispecific construct of claim 1, wherein the anti-B cell binding arm comprises a first complementarity determining region (CDR) H₁ (CDRH₁), a first CDRH₂, a first CDRH₃, a first CDRL₁, a first CDRL₂, and a first CDRL₃ which are obtained from an anti-CD20 antibody, and the second binding arm comprises a second CDRH₁, a second CDRH₂, a second CDRH₃, a second CDRL₁, a second CDRL₂, and a second CDRL₃ which are obtained from an anti-CD68 antibody.

4. The multispecific construct of claim 3, wherein the anti-CD20 antibody is selected from the group consisting of rituximab, obinutuzumab, ofatumumab, ocrelizumab, veltuzumab, ocaratuzumab, tositumomab, ublituximab, ibritumomab, PRO131921, 7D8, 2F2, 11B8, and 2C6.

5. The multispecific construct of claim 3, wherein the anti-CD68 antibody is selected from the group consisting of Y1/82A; CD68/684; KP1; FA-11; EPR20545; EPR23917-164; EPR24100-133; EPR24100-8; SP-251; ED1; 3F7D3; PG-M1; CD68-2501; LAMP4-1830; 815CU17; 514H12; Ki-M7; 298807; Zr302; 3A9A7; OTI8A; OTI3b6; UMAB150; SN07-27; LAMP4-824; and C68-2908R.

6. The multispecific construct of claim 1, wherein the anti-B cell binding arm comprises a heavy chain variable sequence and a light chain variable sequence of an anti-CD20 antibody, and the second binding arm comprises a heavy chain variable sequence and a light chain variable sequence of an anti-CD68 antibody.

7. The multispecific construct of claim 6, wherein the anti-CD20 antibody is selected from the group consisting of rituximab, obinutuzumab, ofatumumab, ocrelizumab, veltuzumab, ocaratuzumab, tositumomab, ublituximab, ibritumomab, PRO131921, 7D8, 2F2, 11B8, and 2C6.

8. The multispecific construct of claim 6, wherein the anti-CD68 antibody is selected from the group consisting of Y1/82A; CD68/684; KP1; FA-11; EPR20545; EPR23917-164; EPR24100-133; EPR24100-8; SP-251; ED1; 3F7D3; PG-M1; CD68-2501; LAMP4-1830; 815CU17; 514H12; Ki-M7; 298807; Zr302; 3A9A7; OTI8A; OTI3b6; UMAB150; SN07-27; LAMP4-824; and C68-2908R.

9. The multispecific construct of claim 1, wherein the anti-B cell binding arm and second binding arm are chimeric, human, partially humanized, fully humanized, or semi-synthetic.

10. The multispecific construct of claim 1, wherein the multispecific construct is selected from the group consisting of a full-length antibody, a F(ab′)₂, a chemically-linked F(ab′)₂, a tandem scFv, a diabody (Db), a single chain diabody (scDb), a tandem diabody, a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, and a heteroconjugate antibody.

11. The multispecific construct of claim 1, comprising a chimeric antigen receptor (CAR) T-cell.

12. The multispecific construct of claim 1, wherein the multispecific construct is a bispecific construct.

13. A method of treating an autoimmune disorder and/or disease in a subject in need of such therapy, comprising administering to the subject the multispecific construct of claim 1, wherein the autoimmune disorder and/or disease is selected from the group consisting of Systemic lupus erythematosus (SLE), SLE flare-ups, lupus nephritis (LN), acute Graft versus Host Disease (GvHD), chronic GvHD, Rheumatoid Arthritis (RA), Pemphigus, e.g., Pemphigus vulgaris (PVu), Pemphigus vegetans (PVe), Pemphigus erythematosus (PE), and Pemphigus foliaceus (PF), Chronic Lymphocytic Leukemia (CLL), Granulomatosis with Polyangiitis (GPA), Microscopic Polyangiitis (MPA), Multiple Sclerosis (MS), Primary Progressive Multiple Sclerosis (PPMS), Relapsing Multiple Sclerosis (RMS), Anti-neutrophil Cytoplasmic Antibody (ANCA)-associated vasculitis, Non-Hodgkin's Lymphoma (NHL), Rassmussen's encephalitis, Immune Thrombocytopenic Purpura (ITP), Follicular Lymphoma Leukemia (FLL), and other immune conditions treatable by B cell depletion therapies (BCDT), Inflammatory Bowel Disease (IBD), Ulcerative colitis (UC), Crohn's disease (CD), Pediatric Acute-onset Neuropsychiatric Syndrome (PANS), and Pediatric Autoimmune Neuropsychiatric Disorder Associated with Streptoccocal Infections (PANDAS).

14-18. (canceled)

19. A method of treating a lupus flare-up in a subject who has Systemic lupus erythematosus (SLE), comprising:
(a) analyzing a blood sample from the subject to determine a test quantity of CD68⁺ naïve B cells in the blood sample;
(b) comparing the test quantity of CD68⁺ naïve B cells to a control quantity of CD68⁺ naïve B cells obtained from a cohort of healthy subjects or to a predictive quantity CD68⁺ naïve B cells predictive of a flare-up;
(c) predicting that the subject will experience a lupus flare-up when (1) the test quantity of CD68⁺ naïve B cells (1) exceeds the control quantity of CD68⁺ naïve B cells, or (2) is equal to or exceeds the predictive quantity CD68⁺ naïve B cells; and
(d) treating the subject with the multispecific construct of claim 1 when the subject is predicted to experience a lupus flare-up.

20. The method of claim 19, wherein the CD68⁺ naïve B cells are determined to be positive for CD20 and IgD, and negative for CD27.

21. The method of claim 19, wherein the CD68⁺ naïve B cells are positive for transcription factor ARID3a.

22. The method of claim 19, wherein the CD68⁺ naïve B cells produce autoantibodies.

23. The multispecific construct of claim 6, wherein the anti-CD20 antibody is rituximab and the anti-CD68 antibody is selected from the group consisting of Y1/82A, CD68/684, and KP1.

24. The method of claim 13, wherein the autoimmune disorder and/or disease is selected from the group consisting of SLE, SLE flare-ups, and LN.

25. The method of claim 13, wherein the autoimmune disorder and/or disease is selected from the group consisting of PANS and PANDAS.
